(19) United States
(12) Patent Application Publication
(10) Pub. No.: US 2008/0249091 A1
(43) Pub. Date: Oct. 9, 2008

(54) Indoles Useful in the Treatment of Inflammation

(76) Inventors: Benjamin Pelcman, Solna (SE); Kristofer Olofsson, Solna (SE); Martins Katkevics, Riga (LV); Vita Ozola, Riga (LV); Edgars Suna, Riga (LV); Ivars Kalvins, Riga (LV); Peteris Trapencieris, Riga (LV)

Correspondence Address: MORGAN LEWIS & BOCKIUS LLP, 1111 PENNSYLVANIA AVENUE NW, WASHINGTON, DC, 20004, US

(21) Appl. No.: 11/795,627

(22) PCT Filed: Dec. 22, 2005
(86) PCT No.: PCT/GB05/04978

§ 371 Date: Mar. 25, 2008

Related U.S. Application Data
(60) Provisional application No. 60/644,553, filed on Jan. 19, 2005.
(60) Provisional application No. 60/644,554, filed on Jan. 19, 2005.

(51) Int. Cl. A61K 31/5377; C07D 209/42; A61K 31/404; C07D 413/02; A61P 29/00; A61P 11/00; C07D 401/02; A61K 31/4439

(52) U.S. Cl. 514/235.2; 548/491; 514/419; 546/278.1; 514/339; 544/143

(57) ABSTRACT

There is provided compounds of formula I,

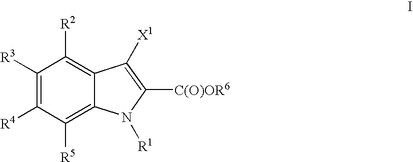

wherein X¹, R¹, R², R³, R⁴, R⁵ and R⁶ have meanings given in the description, and pharmaceutically-acceptable salts thereof, which compounds are useful in the treatment of diseases in which inhibition of the activity of a member of the MAPEG family is desired and/or required, and particularly in the treatment of inflammation.

### FIELD OF THE INVENTION

This invention relates to novel pharmaceutically-useful compounds, which compounds are useful as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). The compounds are of potential utility in the treatment of inflammatory diseases including respiratory diseases. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardioavascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites of arachidonic acid, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B4 is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P.-J. Jacobsson et al in Am. J. Respir. Crit. Care Med 161, S20 (2000). It is well known that compounds prepared as antagonists to one of the MAPEGs may also exhibit inhibitory activity towards other family members, c.f. J. H Hutchinson et al in J. Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

### PRIOR ART

Indole-based compounds have been disclosed in international patent applications WO 96/03377, WO 01/00197, WO 03/044014 and WO 03/057670, U.S. Pat. Nos. 5,189,054, 5,294,722 and 4,960,786 and European patent applications EP 429 257, EP 483 881, EP 547 556, EP 639 573 and EP 1 314 733. In particular European patent application EP 488 532 and U.S. Pat. Nos. 5,236,916 and 5,374,615 disclose 1(N)-phenylindole-2-carboxylates as antihypertensive agents and as chemical intermediates. However, none of these documents disclose or suggest the use of such compounds in the treatment of inflammation.

Indoles have also been disclosed for potential use in the treatment of inflammation in international patent applications WO 99/43672, WO 98/08818, WO 99/43654, WO 99/43651, WO 99/05104 and WO 03/029212, European patent application EP 986 666 and U.S. Pat. Nos. 6,500,853 and 6,630,496. However, there is no specific disclosure in any of these documents of indole-2-carboxylates in which an aromatic group is directly attached via the indole nitrogen.

International patent application WO 01/30343, and European patent application EP 186 367, also mention indoles for potential use as PPAR-Ê binding agents, and in the treatment of inflammation, respectively. However, these documents do not mention or suggest compounds in which the benzenoid moiety of the indole is either substituted with an aromatic ring or directly substituted with a cycloalkyl or heterocycloalkyl ring. Further, propinski et al, Bioorganic and Medicinal Chemistry Letters, 15 (2005) 5035-5038 discloses various indoles for use as PPAR-Ê partial agonists. There is no mention or suggestion of the use of such compounds as inhibitors of mPGES in that document.

Various 1(N)-benzylindole-2-carboxylates and derivatives thereof are known from international patent applications WO 99/33800 as Factor Xa inhibitors; WO 99/07678, WO 99/07351, WO 00/46198, WO 00/46197, WO 00/46195 and WO 00/46199 as inhibitors of MCP-1; international patent application WO 96/18393 as inhibitors of IL-8; international patent applications WO 93/25546 and WO 94/13662, European patent application EP 535 924 A1 and U.S. Pat. No. 5,081,138 as inhibitors of leukotriene biosynthesis; international patent application WO 02/30895 as PPAR-Ê binding agents; and European patent application EP 166 591 as prostaglandin antagonists. Further, international patent application WO 2005/005415 discloses such compounds for use as inhibitors of mPGES and thus in the treatment of inflammation. However, there is no specific disclosure in any of these documents of indole-2-carboxylates in which an aromatic group is directly attached via the indole nitrogen.

Further, unpublished international patent applications PCT/GB2005/002404, PCT/GB2005/002391 and PCT/GB2005/002396 disclose indoles for use as inhibitors of mPGES and thus in the treatment of inflammation. However, there is no suggestion of indoles which are substituted at the benzenoid moiety of the indole with either a cycloalkyl or heterocycloalkyl group or with a aromatic group that is attached via a linking group.

Finally, international patent application WO 94/14434 discloses structurally similar indoles as endothelin receptor antagonists. There is no specific disclosure in this document of indole-2-carboxylates in which an aromatic group is directly attached via the indole nitrogen, nor of compounds in which aromatic and heteroaromatic moieties are attached, via a linking group, or cycloalkyl and heterocycloalkyl moieties are attached, to the benzenoid part of the indole.

### DISCLOSURE OF THE INVENTION

According to the invention there is provided a compound of formula I,

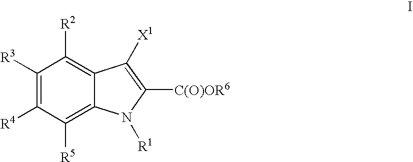

wherein
one of the groups R², R³, R⁴ and R⁵ represents -D-E, a cycloalkyl group or a heterocycloalkyl group (which latter two groups are optionally substituted by one or more substituents selected from G¹ and/or Z¹) and:
a) the other groups are independently selected from hydrogen, G¹, C₁₋₈ alkyl and a heterocycloalkyl group (which latter two groups are optionally substituted by one or more substituents selected from G¹ and/or Z¹), and, in the case when one of R², R³, R⁴ and R⁵ represents -D-E, an aryl group and a heteroaryl group (which latter two groups are optionally substituted by one or more substituents selected from A); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, OR⁶ and ═O;
D represents —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R⁹)-J-R¹⁰ or -Q-X²;
J represents a single bond, —C(O)— or —S(O)ₘ—;
Q represents a single bond, —O—, —C(O)— or —S(O)ₘ;
m represents, on each occasion when mentioned above, 0, 1 or 2;
X² represents:
(a) an aryl group or a heteroaryl group, both of which are optionally substituted by one or more substituents selected from A; or
(b) C₁₋₈ alkyl or a heterocycloalkyl group, both of which are optionally substituted by one or more substituents selected from G¹ and/or Z¹;
R⁶, R⁹ and R¹⁰ independently represent, on each occasion when mentioned above:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
R⁹ and R¹⁰ may be linked together to form, along with the N atom and the J group to which R⁹ and R¹⁰ are respectively attached, a 3- to 8-membered ring, optionally containing 1 to 3 heteroatoms and/or 1 to 3 double bonds, which ring is optionally substituted by one or more substituents selected from G¹ and/or Z¹;
A represents, on each occasion when mentioned above:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when mentioned above, halo, cyano, —N₃, —NO₂, —ONO₂ or -A¹-R^{11a};
wherein A¹ represents a single bond or a spacer group selected from —C(O)A²-, —S(O)₂A³-, —N(R^{12a})A⁴ or —OA⁵-, in which:
A² represents a single bond, —O—, —N(R^{12b}) or —C(O)—;
A³ represents a single bond, —O— or —N(R^{12c})—;
A⁴ and A⁵ independently represent a single bond, —C(O)—, —C(O)N(R^{12d})—, —C(O)O—, —S(O)₂— or —S(O)₂N(R^{12e})—;
Z¹ represents, on each occasion when mentioned above, ═O, ═S, ═NOR^{11b}, ═NS(O)₂N(R^{12f})R^{11c}, ═NCN or ═C(H)NO₂;
B represents, on each occasion when mentioned above:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when mentioned above, halo, cyano, —N₃, —NO₂, —ONO₂ or -A⁶-R^{13a};
wherein A⁶ represents a single bond or a spacer group selected from —C(O)A⁷-, —S(O)₂A⁸-, —N(R^{14a})A⁹- or —OA¹⁰-, in which:
A⁷ represents a single bond, —O—, —N(R^{14b})— or —C(O)—;
A⁸ represents a single bond, —O— or —N(R^{14c})—;
A⁹ and A¹⁰ independently represent a single bond, —C(O)—, —C(O)N(R^{14d})—, —C(O)O—, —S(O)₂— or —S(O)₂N(R^{14e})—;
Z² represents, on each occasion when mentioned above, ═O, ═S, ═NOR^{13b}, ═NS(O)₂N(R^{14f})R^{13c}, ═NCN or ═C(H)NO₂;
R^{11a}, R^{11b}, R^{11c}, R^{12a}, R^{12b}, R^{12c}, R^{12d}, R^{12e}, R^{12f}, R^{13a}, R^{13b}, R^{13c}, R^{14a}, R^{14b}, R^{14c}, R^{14d}, R^{14e} and R^{14f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by G³ and/or Z³; or
any pair of R^{11a} to R^{11c} and R^{12a} to R^{12f}, and/or R^{13a} to R^{13c} and R^{14a} to R^{14f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when mentioned above, halo, cyano, —N₃, —NO₂, —ONO₂ or -A¹¹-R^{15a};
wherein A¹¹ represents a single bond or a spacer group selected from —C(O)A¹²-, —S(O)₂A¹³-, —N(R^{16a})A¹⁴- or —OA¹⁵-, in which:
A¹² represents a single bond, —O—, —N(R^{16b}) or —C(O)—;
A¹³ represents a single bond, —O— or —N(R^{16c});
A¹⁴ and A¹⁵ independently represent a single bond, —C(O)—, —C(O)N(R^{16d})—, —C(O)O—, —S(O)₂— or —S(O)₂N(R^{16e})—;
Z³ represents, on each occasion when mentioned above, ═O, ═S, ═NOR^{15b}, ═NS(O)₂N(R^{16f})R^{15c}, ═NCN or ═C(H)NO₂;
R^{15a}, R^{15b}, R^{15c}, R^{16a}, R^{16b}, R^{16c}, R^{16d}, R^{16e} and R^{16f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{17a})R^{18a}, —OR^{17b} and ═O; and
iii) an aryl or heteroaryl group, both of which are optionally substituted by one or more substituents selected from halo, C₁₋₄ alkyl, —N(R^{17c})R^{18b} and —OR^{17d}; or any pair of R^{15a} to R^{15c} and R^{16a} to R^{16f} may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{17e})R^{18c}, —OR^{17f} and ═O;
R^{17a}, R^{17b}, R^{17c}, R^{17d}, R^{17e}, R^{17f}, R^{18a}, R^{18b} and R^{18c} are independently selected from hydrogen and C₁₋₄ alkyl, which latter group is optionally substituted by one or more halo groups;
or a pharmaceutically-acceptable salt thereof,
provided that, when R³ represents -D-E, in which D represents —C(R⁷)(R⁸)—, X¹, R², R⁴, R⁵, R⁷ and R⁸ all represent H and:
(a) E represents a 2-butyl-5-hydroxymethyl-1H-imidazol-1-yl group, then R⁶ does not represent H when R¹ represents phenyl or 2-carboxyphenyl;
(b) E represents a 2-butyl-5-hydroxymethyl-1H-imidazol-1-yl group or a 2-butyl-5-formyl-1H-imidazol-1-yl group, then R⁶ does not represent ethyl when R¹ represents phenyl or 2-ethoxycarbonylphenyl;
(c) E represents a 2-butyl-4-chloro-5-hydroxymethyl-1H-imidazol-1-yl group, then R⁶ does not represent H or ethyl when R¹ represents 2-(1H-tetrazol-5-yl)phenyl; or
(d) E represents a 2-butyl-4-chloro-5-hydroxymethyl-1H-imidazol-1-yl group or a 2-butyl-4-chloro-5-formyl-1H-imidazol-1-yl group, then R⁶ does not represent ethyl when R¹ represents 2-cyanophenyl,
which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range) defined herein may be straight-chain or, when there is a sufficient number (i.e. a minimum of two or three, as appropriate) of carbon atoms, be branched-chain, and/or, in the case of alkyl, cyclic (so forming a C_{3-q} cycloalkyl group). Further, when there is a sufficient number (i.e. a minimum of four) of carbon atoms, such groups may also be part cyclic. Such alkyl and alkylene groups may also be saturated or, when there is a sufficient number (i.e. a minimum of two) of carbon atoms, be unsaturated (forming, for example, in the case of alkyl, a C_{2-q} alkenyl or a C_{2-q} alkynyl group or, in the case of alkylene, a C_{2-q} alkenylene or a C_{2-q} alkynylene group).

Cycloalkyl groups that may be mentioned include non-aromatic C₃₋₁₆, such as C₃₋₁₀, cycloalkyl groups. C_{3-q} cycloalkyl groups (where q is the appropriate upper limit of the range) may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double or triple bond (forming for example a C_{3-q} cycloalkenyl or a C_{8-q} cycloalkynyl group). Cycloalkyl groups that may be mentioned include cyclopropyl, cyclobutyl, cyclopentyl, cyclohexyl, cycloheptyl, cyclooctyl, cyclopentenyl, cyclohexenyl, cycloheptenyl, cyclooctenyl, cyclooctynyl, bicycloheptyl, bicyclooctyl, and bicyclooctenyl, as well as bridged cycloalkyl groups, such as adamantyl, noradamantyl, norbornane, norbornene and norbornadiene groups. Substituents may be attached at any point on the cycloalkyl group. Further in the case where the substituent is another cyclic compound, then the cyclic substituent may be attached through a single atom on the cycloalkyl group, forming a so-called “spiro”-compound. Preferred cycloalkyl groups include optionally substituted C₃₋₈ cycloalkyl groups, which groups optionally contain one unsaturation (e.g. a double bond). Cycloalkyl groups that may be mentioned include optionally substituted cyclopropyl, cyclobutyl, cyclopentyl, cyclohexyl, cyclopentenyl (e.g. cyclopenten-1-yl), cyclohexenyl (e.g. cyclohexen-1-yl) and norbornanyl (e.g. norbornan-2-yl).

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the other substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. When, for example, one of R², R³, R⁴ and R⁵ represents a cycloalkyl or a heterocycloalkyl group, preferred heterocycloalkyl groups include optionally substituted 5 to 6-membered heterocyclic groups containing at least one oxygen or, more preferably, nitrogen atom and, optionally, a further nitrogen and/or oxygen atom. Heterocycloalkyl groups that may be mentioned include optionally substituted pyrrolidinyl (e.g. pyrrolidin-1-yl), morpholinyl (e.g. 4-morpholin-1-yl), piperazinyl (e.g. piperazin-1-yl), piperidinyl (e.g. piperidin-1-yl and piperidin-4-yl) and tetrahydropyridyl (e.g. 1,2,3,6-tetrahydropyridin-2-yl) groups.

For the avoidance of doubt, the term “bicyclic”, when employed in the context of cycloalkyl and heterocycloalkyl groups refers to such groups in which the second ring is formed between two adjacent atoms of the first ring. The term “bridged”, when employed in the context of cycloalkyl or heterocycloalkyl groups refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic, bicyclic or tricyclic and have between 6 and 14 ring carbon atoms, in which at least one, ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which R¹ and X² are both aryl groups substituted by one or more C₁₋₈ alkyl groups, the alkyl groups in question may be the same or different. Similarly, when groups are substituted by more than one substituent as defined herein, the identities of those individual substituents are not to be regarded as being interdependent. For example, when X² and/or R¹ represents e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R² to R⁵” is employed herein, this will be understood by the skilled person to mean R², R³, R⁴ and R⁵ inclusively.

As stated hereinbefore, any pair of R^{11a} to R^{11c} and R^{12a} to R^{12f}, may be linked as hereinbefore defined. For the avoidance of doubt, such R^{11a} to R^{11c} groups, and R^{12a} to R^{12f} groups may be attached to a single nitrogen atom (e.g. R^{11a} and R^{12a} or R^{11c} and R^{12f}), which may form part of the ring.

Compounds of the invention that may be mentioned include those in which one of the groups R², R³, R⁴ and R⁵ represents -D-E as hereinbefore defined.

Compounds of the invention that may be mentioned also include those in which one of the groups R², R³, R⁴ and R⁵ represents a cycloalkyl group or a heterocycloalkyl group, both of which are optionally substituted as hereinbefore defined.

Compounds of the invention that may be mentioned include those in which when one of the groups R², R³, R⁴ and R⁵ represents -D-E then:

a) the other groups are independently selected from hydrogen, G¹, C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two atoms of the essential benzene ring in the compound of formula I, a 3- to 8-membered ring, optionally containing 1 to 3 heteroatoms and/or 1 to 3 double bonds, which ring is itself optionally substituted by one or more substituents selected from halo, —R⁶, —OR⁶ and ═O.

Further compounds of the invention that may be mentioned include those in which, when one of the groups R², R³, R⁴ and R⁵ represents optionally substituted cycloalkyl or heterocycloalkyl as hereinbefore defined or, more particularly, -D-E and one or more of the other groups represent G¹ then, when G¹ represents -A¹-R^{11a}, A¹ represents a single bond, then R^{11a} represents hydrogen, C₁₋₈ alkyl or a heterocycloalkyl group (which latter two groups are optionally substituted by one or more substituents selected from G³ and/or Z³).

Yet further compounds of the invention that may be mentioned include those in which, when one of the groups R², R³, R⁴ and R⁵ represents optionally substituted cycloalkyl or heterocycloalkyl as hereinbefore defined or, more particularly, -D-E and one or more of the other groups represent G¹ then, when G¹ represents -A¹-R^{11a}, A¹ represents a spacer group selected from —C(O)A²-, —S(O)₂A³-, —N(R^{12a})A⁴- or —OA⁵-.

Compounds of the invention that may be mentioned also include those in which, for example, when one of the groups R², R³, R⁴ and R⁵ represents optionally substituted cycloalkyl or heterocycloalkyl as hereinbefore defined or, more particularly, -D-E, and X¹ represents -Q-X², Q is a single bond and X² is either:

(a) an aryl group or a heteroaryl group, which groups are substituted by A in which A is G¹; or
(b) C₁₋₈ alkyl or a heterocycloalkyl group, which groups are substituted by G¹, and, in either case, G¹ is -A¹-R^{11a}, then A¹ represents a single bond or a spacer group selected from —C(O)—, —S(O)₂—, —S(O)₂N(R^{12c})—, —N(R^{12a})A⁴- or —OA⁵-.

Further compounds of the invention that may be mentioned include those in which, when one of the groups R², R³, R⁴ and R⁵ represents optionally substituted cycloalkyl or heterocycloalkyl as hereinbefore defined or, more particularly, -D-E, and X¹ represents -Q-X², Q is a single bond, X² is C₁₋₈ alkyl substituted by G¹, G¹ is -A¹-R^{11a}, A¹ is a single bond, R^{11a} represents an aryl group, a heteroaryl group or a heterocycloalkyl group, all of which groups are substituted by G³, and G³ is -A¹¹-R^{15a}, then A¹¹ represents a single bond or a spacer group selected from —C(O)—, —S(O)₂—, —S(O)₂N(R^{16c})—, —N(R^{16a})A¹⁴- or —OA¹⁵-.

Yet further compounds of the invention that may be mentioned include those in which when one of the groups R², R³, R⁴ and R⁵ represents optionally substituted cycloalkyl or heterocycloalkyl as hereinbefore defined or, more particularly, -D-E and X² represents C₁₋₈ alkyl terminally substituted by both Z¹ and G¹, in which Z¹ represents ═O and G¹ represents -A¹-R^{11a}, then when A¹ represents —N(R^{12a})A⁴-, A⁴ represents —C(O)—, —C(O)N(R^{12d})—, —C(O)O— or —S(O)₂N(R^{12e}), and when A¹ represents —OA⁵-, A⁵ represents —C(O)—, —C(O)N(R^{12d})—, —C(O)O—, —S(O)₂— or —S(O)₂N(R^{12e}).

Further compounds of the invention that may be mentioned include those in which, when R³ represents -D-E, in which D represents —C(R⁷)(R⁸)— and R⁷ and R⁸ both represent H, then E does not represent an optionally substituted imidazolyl (e.g. imidazol-1-yl) group, and particularly an optionally substituted 2-butyl-1H-imidazol-1-yl (such as a 2-butyl-5-hydroxymethyl-1H-imidazol-1-yl, 2-butyl-5-formyl-1H-imidazol-1-yl, 2-butyl-4-chloro-5-hydroxymethyl-1H-imidazol-1-yl, or a 2-butyl-4-chloro-5-formyl-1H-imidazol-1-yl, group).

Still further compounds of the invention that may be mentioned include those in which:

(i) when R³ represents -D-E, in which D represents —C(R⁷)(R⁸)—, R⁷ and R⁸ do not both represent H when E represents a heteroaryl group;
(ii) when R³ represents -D-E, in which D represents —C(R⁷)(R⁸)—, R⁷ and R⁸ do not both represent H;
(iii) when D represents —C(R⁷)(R⁸)—, R⁷ and R⁸ do not both represent H.

Yet further compounds of the invention that may be mentioned include those in which D represents C₂₋₄ alkylene or, more preferably, —O—, —C(O)— or —S(O)ₘ—.

Preferred compounds of the invention include those in which:

when one of R² to R⁵ represents an optionally substituted cycloalkyl or heterocycloalkyl group, then it is preferably R³ or R⁴;
Q represents —O—, —S— or, more preferably, a single bond;
A represents C₁₋₆ alkyl optionally substituted by one or more G¹ groups or (more preferably, in the case where one of R² to R⁵ represents a cycloalkyl or heterocycloalkyl group) G¹;
X² represents C₁₋₆ (e.g. C₁₋₄) alkyl or heterocycloalkyl, both of which are optionally substituted (and preferably substituted in the case where one of R² to R⁵ represents a cycloalkyl or heterocycloalkyl group) by one or more (e.g. one) G¹ and/or Z¹ groups;
R⁹ represents H or C₁₋₂ alkyl (e.g. methyl);
R¹⁰ represents heteroaryl or, preferably, C₁₋₆ (such as C₁₋₄ (e.g. C₁₋₃)) alkyl, which group may be unsubstituted or is (e.g. preferably) substituted by one or more (e.g. one) groups selected from G¹;
R⁹ and R¹⁰ are linked to form a 4- to 7-membered (e.g. 5- or 6-membered) ring, which ring may, for example preferably, contain (in addition to the nitrogen atom to which R⁹ is attached) a further heteroatom (e.g. nitrogen or oxygen) and which ring is optionally substituted by one or more (e.g. two) Z¹ groups;
G¹ represents halo, cyano, —NO₂ or -A¹-R^{11a};
when one of R² to R⁵ represents -D-E-, then A¹ represents a single bond, —C(O)A²-, —N(R^{12a})A⁴- or, preferably, —OA⁵-;
when one of R² to R⁵ represents an optionally substituted cycloalkyl or heterocycloalkyl group, then A¹ represents —N(R^{12a})A⁴- or, more preferably, a single bond, —C(O)A²- or —OA⁵-;
A² represents —O— or, in the case where one of R² to R⁵ represents an optionally substituted cycloalkyl or heterocycloalkyl group, —N(R^{12b})—;
A⁴ and A⁵ independently represent —C(O)—, —C(O)N(R^{12d})—, —C(O)O— or, preferably in the case where any one of R² to R⁵ represents an optionally substituted cycloalkyl or heterocycloalkyl group, a single bond;
Z¹ represents ═NOR^{11b}, ═NCN or, preferably, ═O;
when one of R² to R⁵ represents -D-E-, then R^{11a}, R^{11b} and R^{11c} independently represent hydrogen, an aryl group, a heteroaryl group, a heterocycloalkyl group (such as C₄₋₈ heterocycloalkyl, which group contains one oxygen or, more preferably, nitrogen atom and, optionally, a further nitrogen or oxygen atom) or, preferably, C₁₋₆ (e.g. C₁₋₄) alkyl, which latter four groups are optionally substituted by one or more G³ groups and/or (in the case of alkyl and heterocycloalkyl) Z³ groups;
when one of R² to R⁵ represents an optionally substituted cycloalkyl or heterocycloalkyl group, then R^{11a}, R^{11b} and R^{11c} independently represent aryl or, preferably, H or, more preferably, C₁₋₇ alkyl, C₄₋₈ heterocycloalkyl (which heterocycloalkyl group contains one oxygen or, more preferably, nitrogen atom and, optionally, a further nitrogen or oxygen atom) or a heteroaryl group, which latter three groups are optionally substituted by one or more G³ groups and/or (in the case of alkyl and heterocycloalkyl) Z³ groups;
R^{12a}, R^{12b}, R^{12c}, R^{12d}, R^{12e} and R^{12f} independently represent H or (more preferably, in the case where one of R² to R⁵ represents an optionally substituted cycloalkyl or heterocycloalkyl group) C₁₋₂ alkyl;
G² represents cyano, —N₃ or, more preferably, halo, —NO₂ or -A⁶-R^{13a};
A⁶ represents —N(R^{14a})A⁹- or —OA¹⁰-;
A⁹ represents —C(O)N(R^{14d})—, —C(O)O— or, more preferably, a single bond or —C(O)—;
A¹⁰ represents a single bond;
Z² represents ═NOR^{13b} or ═NCN or, more preferably, ═O;
G³ represents halo, —NO₂ or -A¹¹-R^{15a};
A¹¹ represents —N(R^{16a})— or —O—;
Z³ represents ═O;
J represents a single bond or, preferably, —C(O)— or —S(O)₂—;
when any one of R^{15a}, R^{15b}, R^{15c}, R^{16a}, R^{16b}, R^{16c}, R^{16d}, R^{16e} and R^{16f} represents optionally substituted C₁₋₆ alkyl, the optional substituent is one or more halo groups;
when any one of R^{17a}, R^{17b}, R^{17c}, R^{17d}, R^{17e}, R^{17f}, R^{18a}, R^{18b} and R^{18c} represents optionally substituted C₁₋₄ alkyl, the optional substituent is one or more fluoro groups.

Preferred aryl and heteroaryl groups that R¹, E and (when they represent such aryl or heteroaryl groups) X², R⁹ and R¹⁰ may represent include optionally substituted phenyl, naphthyl, pyrrolyl, furanyl, thienyl, pyrazolyl, imidazolyl (e.g. 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

Preferred values of R¹ include optionally substituted phenyl, pyridyl (e.g. 2-pyridyl or 3-pyridyl) and imidazolyl.

Preferred values of E include optionally substituted 1,3-benzodioxolyl (e.g. 1,3-benzodioxol-5-yl), preferably, pyridyl (e.g. 2- or 3-pyridyl), imidazolyl, more preferably quinolinyl (e.g. 3-quinolinyl), and particularly phenyl.

Optional substituents on R¹, R², R³, R⁴, R⁵, X² and E groups are preferably selected from:

aryl (e.g. phenyl);
in the case of substituents on non-aromatic groups (e.g. cycloalkyl or heterocycloalkyl groups), ═O; or, more preferably,
halo (e.g. fluoro, chloro or bromo);
cyano;

### —NO₂;

C₁₋₆ alkyl, which alkyl group may be linear or branched (e.g. C₁₋₄ alkyl (including ethyl, n-propyl, isopropyl, n-butyl or, preferably, methyl or t-butyl), n-pentyl, isopentyl, n-hexyl or isohexyl), cyclic (e.g. cyclopropyl, cyclobutyl, cyclopentyl or cyclohexyl), part-cyclic (e.g. cyclopropylmethyl), unsaturated (e.g. 1-propenyl, 2-propenyl, 1-butenyl, 2-butenyl, 3-butenyl, 1-pentenyl, 2-pentenyl, 4-pentenyl or 5-hexenyl) and/or optionally substituted with one or more halo (e.g. fluoro) group (so forming, for example, fluoromethyl, difluoromethyl or, preferably, trifluoromethyl);
heterocycloalkyl, such as a C₄₋₅ heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR¹⁹;

### —N(R¹⁹)R²⁰; and

in the case where one of R² to R⁵ represents an optionally substituted cycloalkyl or heterocycloalkyl group, —C(O)OR¹⁹;
wherein R¹⁹ and R²⁰ independently represent, on each occasion when mentioned above, H or C₁₋₆ alkyl, such as methyl, ethyl, n-propyl, isopropyl, n-butyl, t-butyl and, preferably, in the case where one of R² to R⁵ represents -D-E, methyl or isopropyl and, in the case where one of R² to R⁵ represents an optionally substituted cycloalkyl or heterocycloalkyl group, isopropyl or t-butyl (which alkyl groups are optionally substituted by one or more halo (e.g. fluoro) groups (to form e.g. a trifluoromethyl group)).

Preferred values of R⁶ include C₁₋₄ alkyl and, particularly, H.

More preferred compounds of the invention include those in which, when one of R² to R⁵ represents -D-E-, then:

one of R⁴ and, more preferably, R³ represents -D-E and the other (more preferably) represents H;
D represents —CH₂—, preferably ethylene (e.g. ethynylene), —S—, —S(O)—, —S(O)₂— or, more preferably, —O— or —C(O)—;
X¹ represents —N(R⁹)-J-R¹⁰ or, more preferably, C₁₋₃ alkyl (e.g. methyl), heterocycloalkyl (which latter two groups are optionally substituted by G¹ and, preferably, —N(R^{12a})R^{11a}, —OR^{11a}, —R^{11a} or halo (e.g. fluoro or chloro)), H or halo (e.g. fluoro or chloro);
R² represents chloro or, preferably H;
R⁵ represents H;
A represents G¹, or C₁₋₆ (e.g. C₁₋₄) alkyl (e.g. cyclohexyl or, preferably, methyl or t-butyl) optionally substituted by one or more G¹ groups;
G¹ represents cyano or, preferably, fluoro, chloro, —NO₂ or -A¹-R^{11a};
A⁴ represents —C(O)— or, preferably, a single bond;
A⁵ represents a single bond;
R⁹ represents H or methyl,
R¹⁰ represents methyl, t-butyl, pyridyl (e.g. 3-pyridyl), propyl (e.g. n-propyl optionally substituted by a G¹ (e.g. —N(R^{12a})R^{11a}) group); or
R⁹ and R¹⁰ are linked to form a 5- or 6-membered (e.g. 5-membered) ring, which is substituted by one Z¹ group;
R^{11a}, R^{11b} and R^{11c} independently represent a phenyl group, a heteroaryl (such as tetrazolyl (e.g. 5-tetrazolyl), imidazolyl (e.g. 4-imidazolyl or 2-imidazolyl) or a pyridyl (e.g. 3-pyridyl, 4-pyridyl or, especially, 2-pyridyl)) group, or, more preferably, C₁₋₃ alkyl (e.g. methyl or isopropyl) optionally substituted by one or more G³ groups;
R^{12a}, R^{12b}, R^{12c}, R^{12d}, R^{12e} and R^{12f} independently represent H or methyl;
G³ represents halo (e.g. fluoro).

Further preferred compounds of the invention include those in which, when one of R² to R⁵ represents an optionally substituted cycloalkyl or heterocyclalkyl group as hereinbefore defined, then:

one of R³ and R⁴ represents an optionally substituted cycloalkyl group, or an optionally substituted heterocycloalkyl group, as specified hereinbefore, and the other represents H;
X¹ represents —N(R⁹)-J-R¹⁰, preferably, H, C₁₋₃ alkyl, heterocycloalkyl (which latter two groups are preferably substituted by —N(R^{12a})R^{11a}, —OR^{11a} or —R^{11a}) or, more preferably, halo (e.g. fluoro or, particularly, chloro);
R² and/or R⁵ independently represent H;
A represents G¹;
G¹ represents fluoro, chloro or -A¹-R^{11a};
A² represents —O—;
A⁵ represents a single bond;
R^{11a}, R^{11b} and R^{11c} independently represent an aryl (e.g. phenyl) group or, preferably, a heteroaryl group (such as tetrazolyl (e.g. 5-tetrazolyl) or, more preferably, pyridyl (e.g. 2-pyridyl, 3-pyridyl or 4-pyridyl) or imidazolyl (e.g. 4-imidazolyl or 2-imidazolyl)), more preferably, C₁₋₆ alkyl (e.g. methyl, isopropyl, 1-butyl or cyclopentyl) or C₄₋₆ heterocycloalkyl (e.g. pyrrolidinyl, piperidinyl, piperazinyl and morpholinyl), all of which are optionally substituted by one or more G³ groups;
R^{12a}, R^{12b}, R^{12c}, R^{12d}, R^{12e} and R^{12f} independently represent H or methyl;
G³ represents halo (e.g. fluoro).

Most preferred compounds of the invention that may be mentioned include those in which X¹ groups (e.g. when one of R² to R⁵ represents -D-E) represent H, chloro, —C₂H₅CN, pyrrolidinyl (e.g. 2-oxopyrrolidin-1-yl), —N(CH₃)C(O)CH₃, —N(H)C(O)t-butyl, —N(H)C(O)CH₃, —N(H)C(O)-pyrid-3-yl, —N(H)S(O)₂CH₃, —N(H)C(O)C₃H₆N(CH₃)₂, —N(H)C₃H₆—N(CH₃)₂ or —N(H)C(O)t-butyl.

Values of R¹ that may be mentioned include 4-methyl-3-nitrophenyl, 4-acetamidophenyl or, preferably, 4-cyclopropyloxyphenyl, 4-cyclopentyloxyphenyl and 4-isopropoxyphenyl.

Values of E that may be mentioned include unsubstituted phenyl, isopropoxyphenyl (e.g. 2-, 3 or 4-isopropoxyphenyl), trifluoromethoxyphenyl (e.g. 3- or 4-trifluoromethoxyphenyl), dichlorophenyl (e.g. 3,5- or 3,4-dichlorophenyl), 4-tert-butylphenyl, chlorophenyl (e.g. 4-chlorophenyl), trifluoromethylphenyl (e.g. 3-trifluoromethyphenyl), trifluoromethoxyphenyl (e.g. 3- or 4-trifluoromethoxyphenyl), chloropyridyl (e.g. 6-chloropyrid-3-yl or 6-chloropyrid-2-yl), benzodioxolyl (e.g. 1,3-benzodioxol-5-yl or 2,2-difluoro-1,3-benzodioxol-5-yl), 3-trifluoromethoxy-4-chlorophenyl, 3-trifluoromethoxy-4-isopropoxyphenyl, 3-fluoro-4-trifluoromethoxyphenyl or, preferably, 3-chlorophenyl, 4-trifluoromethylphenyl, 5-trifluoromethoxypyridin-2-yl, 6-trifluoromethoxypyridin-3-yl and 4-cyclohexylphenyl.

Particularly preferred values of cycloalkyl or heterocycloalkyl groups that R² to R⁵ may represent include 1-piperidinyl, 2-phenylcyclopropyl, 5-tert-butyl-2-hydroxycyclohexyl and 5-tert-butyl-2-oxo-cyclohexyl.

Particularly preferred values of X² include C₁₋₃ alkyl (e.g. methyl), which group is unsubstituted or, preferably, substituted by one or more halo (e.g. fluoro or chloro) groups so forming, for example, a trifluoromethyl group.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) reaction of a compound of formula II,

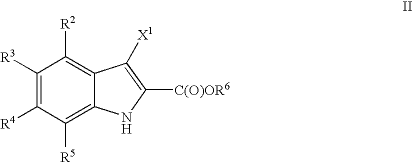

wherein X¹, R², R³, R⁴, R⁵ and R⁶ are as hereinbefore defined, with a compound of formula III,

R¹L¹ III

wherein L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ and R¹ is as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when R¹ represents phenyl and L¹ represents bromo, i.e. bromobenzene). This reaction may be carried out at room temperature or above (e.g. at a high temperature, such as the reflux temperature of the solvent system that is employed) or using microwave irradiation;
(ii) for compounds of formula I in which X¹ represents -Q-X², in which Q is a single bond or —C(O)—, reaction of a compound of formula IV,

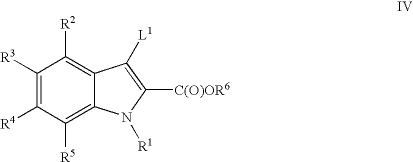

wherein L¹, R¹, R², R³, R⁴, R⁵ and R⁶ are as hereinbefore defined, with a compound of formula V,

X²-Q^{a}-L² V

wherein Q^{a} represents a single bond or —C(O)—, L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo[3.3.1]nonane (9-BBN), —Sn(alkyl)₃ (e.g. —SnMe₃ or —SnBu₃), or a similar group known to the skilled person, and X² is as hereinbefore defined. The skilled person will appreciate that L¹ and L² will be mutually compatible. In this respect, preferred leaving groups for compounds of formula V in which Q^{a} is —C(O)— include chloro or bromo groups, and preferred leaving groups for compounds of formula V in which Q^{a} is a single bond include —B(OH)₂, 4,4,5,5-tetramethyl-1,3,2-dioxaborolan-2-yl, 9-borabicyclo[3.3.1]nonane (9-BBN), or —Sn(alkyl)₃. This reaction may be performed, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)-ethane, 2,2′-bis(di-tert-butylphosphino)-1,1′-biphenyl, 2,2′-bis(diphenylphosphino)-1,1-bi-naphthyl, 1,1′-bis(diphenyl-phosphinoferrocene), 1,3-bis(diphenylphosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation. The skilled person will appreciate that certain compounds of formula IV (in particular those in which L¹ represents chloro, bromo or iodo) are also compounds of formula I and therefore compounds of the invention. In the case where Q^{a} represents a single bond and X² represents either C₂₋₈ alkenyl, cycloalkenyl or heterocycloalkenyl in which the double bond is between the carbon atoms that are α and β to L², the skilled person will appreciate that the double bond may migrate on formation of the compound of formula I to form a double bond that is between the carbon atoms that are β and γ to the indole ring;
(iii) for compounds of formula I in which X¹ represents -Q-X² and Q represents —C(O)—, reaction of a compound of formula I in which X¹ represents H with a compound of formula V in which Q^{a} represents —C(O)— and L² represents a suitable leaving group such as chloro or bromo, —N(C₁₋₆ alkyl)₂ (e.g. —N(CH₃)₂) or a carboxylate group such as —O—C(O)—X^{2y} in which X^{2y} represents X² or H. In the latter case, X^{2y} and X² are preferably the same, or X^{2y} represents e.g. H, CH₃ or CF₃. This reaction may be performed under suitable conditions known to those skilled in the art, for example in the presence of a suitable Lewis acid (e.g. AlCl₃ or FeCl₃). Reaction of a compound of formula V in which L² represents —N(C₁₋₆ alkyl)₂ and X² represents optionally substituted aryl (e.g. phenyl) or heteroaryl may be performed in the presence of a reagent such as POCl₃, for example under reaction conditions described in Bioorg. Med. Chem. Lett., 14, 4741-4745 (2004). The skilled person will appreciate that in the latter instance, POCl₃ may convert the compound of formula V into one in which L² represents chloro and/or Q^{a} represents a derivative of —C(O)— (e.g. an iminium derivative), which group may be transformed back to a —C(O)— group before or after reaction with the compound of formula I in which X¹ represents H;
(iv) for compounds of formula I in which X¹ represents —N(R⁹)-J-R¹⁰ or Q-X² in which Q represents —O— or —S—, reaction of a compound of formula IV as hereinbefore defined with a compound of formula VI,

X^{1b}H VI

in which X^{1b} represents —N(R⁹)-J-R¹⁰ or -Q-X² in which Q represents —O— or —S— and R⁹, J, R¹⁰ and X² are as hereinbefore defined, for example under reaction conditions such as those hereinbefore described in respect of either process (i) or (ii) above;
(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as hereinbefore defined, for example in the presence of N-chlorosuccinimide and a suitable solvent (e.g. dichloromethane), e.g. as described in inter alia Org. Lett., 819-821 (2004). Alternatively, reaction of a compound of formula VI in which X^{1b} represents -Q-X², Q represents —S— and X² represents an optionally substituted aryl (phenyl) or heteroaryl (e.g. 2-pyridyl), group, may be performed in the presence of PIFA (PhI(OC(O)CF₃)₂) in a suitable solvent such as (CF₃)₂CHOH. Introduction of such an —S—X² group is described in inter alia Bioorg. Med. Chem. Lett., 14, 4741-4745 (2004);
(vi) for compounds of formula I in which X¹ represents -Q-X² and Q represents —S(O)— or —S(O)₂—, oxidation of a corresponding compound of formula I in which Q represents —S— under appropriate oxidation conditions, which will be known to those skilled in the art;
(vii) for compounds of formula I in which X¹ represents -Q-X², X² represents C₁₋₈ alkyl substituted by G¹, G¹ represents -A¹-R^{11a}, A¹ represents —N(R^{12a})A⁴- and A⁴ is a single bond (provided that Q represents a single bond when X² represents substituted C₁ alkyl), reaction of a compound of formula VII,

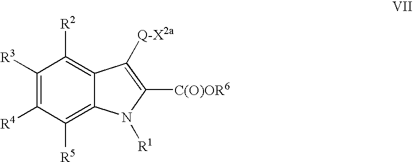

wherein X^{2a} represents a C₁₋₈ alkyl group substituted by a -Z¹, group in which Z¹ represents ═O, Q is as hereinbefore defined, provided that it represents a single bond when X^{2a} represents C₁ alkyl substituted by ═O (i.e. —CHO), and R¹, R², R³, R⁴, R⁵ and R⁶ are as hereinbefore defined under reductive amination conditions in the presence of a compound of formula VIII,

R^{11a}(R^{12a})NH VIII

wherein R^{11a} and R^{12a} are as hereinbefore defined, under conditions well known to those skilled in the art;
(viia) for compounds of formula I in which X¹ represents -Q-X², Q represents a single bond, X² represents methyl substituted by G¹, G¹ represents -A¹-R^{11a}, A¹ represents —N(R^{12a})A⁴-, A⁴ is a single bond and R^{11a} and R^{12a} are preferably methyl, reaction of a corresponding compound of formula I in which X¹ represents H, with a mixture of formaldehyde (or equivalent reagent) and a compound of formula VIII as hereinbefore defined (e.g. in which R^{11a} and R^{12a} represent methyl), for example in the presence of solvent such as a mixture of acetic acid and water, under e.g. standard Mannich reaction conditions known to those skilled in the art;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula IV in which L¹ represents halo (e.g. iodo) with a compound of formula IXA,

H₂C═C(H)X^{2b} IXA

or, depending upon the geometry of the double bond, reaction of a compound of formula VII in which Q represents a single bond and X^{2a} represents —CHO with either a compound of formula IXB,

(EtO)₂P(O)CH₂X^{2b} IXB

or the like, or a compound of formula IXC,

(Ph)₃P═CHX^{2b} IXC

or the like, wherein, in each case, X^{2b} represents H, G¹ or C₁₋₆ alkyl optionally substituted with one or more substituents selected from G¹ and/or Z¹ and G¹ and Z¹ are as hereinbefore defined, for example, in the case of a reaction of a compound of formula IV with compound of formula I×A, in the presence of an appropriate catalyst (such as PdCl₂(PPh₃)₂), a suitable base (e.g. NaOAc and/or triethylamine) and an organic solvent (e.g. DMF) and, in the case of reaction of a compound of formula VII with either a compound of formula IXB, or IXC, under standard Horner-Wadsworth-Emmons, or Wittig, reaction conditions, respectively;
(ix) for compounds of formula I in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction (e.g. hydrogenation) of a corresponding compound of formula I in which X represents optionally substituted C₂₋₈ alkenyl, cycloalkenyl, heterocycloalkenyl, C₂₋₈ alkynyl, cycloalkynyl or heterocycloalkynyl (as appropriate) under conditions that are known to those skilled in the art. For example, in the case where an alkynyl group is converted to a alkenyl group, in the presence of an appropriate poisoned catalyst (e.g. Lindlar's catalyst);
(x) for compounds of formula I in which one or more of R², R³, R⁴ and/or R⁵ represents -D-E, in which D represents —C(O)—, —C(R⁷)(R⁸)—, C₂₋₄ alkylene or —S(O)₂—, or optionally substituted cycloalkyl or heterocycloalkyl, reaction of a compound of formula X,

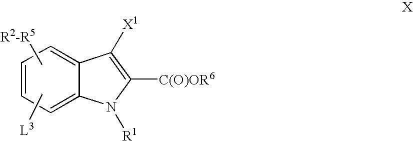

wherein L³ represents L¹ or L² as hereinbefore defined, which group is attached to one or more of the carbon atoms of the benzenoid ring of the indole, R²-R⁵ represents whichever of the three other substituents on the benzenoid ring, i.e. R², R³, R⁴ and R⁵, are already present in that ring, and X¹, R¹, R², R³, R⁴, R⁵ and R⁶ are as hereinbefore defined, with, in the case where one of R² to R⁵ represents -D-E in which D represents —C(O)—, —C(R⁷)(R⁸)—, C₂₋₄ alkylene or —S(O)₂—, a compound of formula XI,

E-D^{a}-L⁴ XI

wherein D^{a} represents —C(O)—, —C(R⁷)(R⁸)— or C₂₋₄ alkylene or —S(O)₂—, L⁴ represents L¹ (when L³ is L²) or L² (when L³ is L¹), and L¹, L², E, R⁷ and R³ are as hereinbefore defined, or, in the case where one of R² to R⁵ represents an optionally substituted cycloalkyl or heterocycloalkyl group, a compound of formula XIA,

(R²⁻⁵)-L⁴ XIA

wherein (R²⁻⁵) represents whichever one of the substituents R², R³, R⁴ or R⁵ is being introduced and L⁴, R², R³, R⁴ and R⁵ are as hereinbefore defined. For example, in the case of reaction with the compound of formula XIA or with the compound of formula XI in which D^{a} represents —C(O)— or C₂₋₄ alkylene, the reaction may be performed for example under similar conditions to those described hereinbefore in respect of process step (ii) above. Further, in the case of reaction with a compound of formula XI in which D^{a} represents —C(O)—, —C(R⁷)(R⁸)—, C₂₋₄ alkylene or —S(O)₂—, the reaction may be performed by first activating the compound of formula X. The skilled person will appreciate that compounds of formula X may first be activated when L³ represents halo, by:
(I) forming the corresponding Grignard reagent under standard conditions known to those skilled in the art (e.g. employing magnesium or a suitable reagent such as a mixture of C₁₋₆ alkyl-Mg-halide and ZnCl₂ or LiCl), followed by reaction with a compound of formula XI or XIA (as appropriate), optionally in the presence of a catalyst (e.g. FeCl₃) under conditions known to those skilled in the art; or
(II) forming the corresponding lithiated compound under halogen-lithium exchange reaction conditions known to those skilled in the art (e.g. employing n-BuLi or t-BuLi in the presence of a suitable solvent (e.g. a polar aprotic solvent such as THF)), followed by reaction with a compound of formula XI or XIA (as appropriate).

The skilled person will also appreciate that the magnesium of the Grignard reagent or the lithium of the lithiated species may be exchanged to a different metal (i.e. a transmetallation reaction may be performed), for example to zinc (e.g. using ZnCl₂) and the intermediate so formed may then be subjected to reaction with a compound of formula XI or XIA (as appropriate) under conditions known to those skilled in the art, for example such as those described hereinbefore in respect of process (ii) above;

(xi) for compounds of formula I in which when one of R² to R⁵ represents -D-E- and D represents —S—, —O— or C₂₋₄ alkynylene in which the triple bond is adjacent to E, reaction of a compound of formula X as hereinbefore defined in which L³ represents L² as hereinbefore defined (for example —B(OH)₂) with a compound of formula XII,

E-D^{b}-H XII

wherein D^{b} represents —S—, —O— or C₂₋₄ alkynylene in which the triple bond is adjacent to E and E is as hereinbefore defined. Such reactions may be performed under similar conditions to those described hereinbefore in respect of process step (ii) above, for example in the presence of a suitable catalyst system, such as Cu(OAc)₂, a suitable base, such as triethylamine or pyridine, and an appropriate organic solvent, such as DMF or dichloromethane;
(xii) for compounds of formula I in which when one of R² to R⁵ represents -D-E- and D represents —S(O)— or —S(O)₂—, oxidation of a corresponding compound of formula I in which D represents —S— under appropriate oxidation conditions, which will be known to those skilled in the art;
(xiii) for compounds of formula I in which when one of R² to R⁵ represents -D-E- and D represents —O— or —S—, reaction of a compound of formula XIII,

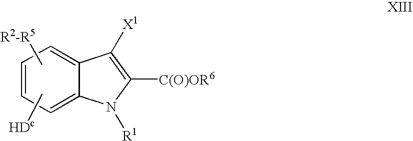

wherein the -D^{c}-H group is attached to one or more of the carbon atoms of the benzenoid ring of the indole, D^{c} represents —O— or —S— and X¹, R¹, R²-R⁵ and R⁶ are as hereinbefore defined, with a compound of formula XIV,

E-L² XIV

wherein L² is as hereinbefore defined (for example —B(OH)₂, chloro, bromo or iodo) and E is as hereinbefore defined, under conditions known to those skilled in the art, for example under conditions such as those described hereinbefore in respect of process step (ii) above;
(xiv) for compounds of formula I in which X¹ represents —N(R⁹)-J-R¹⁰, reaction of a compound of formula XV,

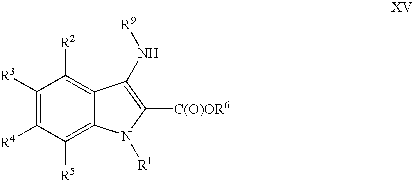

wherein R¹, R², R³, R⁴, R⁵, R⁶ and R⁹ are as hereinbefore defined, with a compound of formula XVI,

R¹⁰-J-L¹ XVI

wherein J, R¹⁰ and L¹ are as hereinbefore defined, for example at around room temperature or above (e.g. up to 60-70° C.) in the presence of a suitable base (e.g. pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, or mixtures thereof), an appropriate solvent (e.g. pyridine, dichloromethane, chloroform, tetrahydrofuran, dimethylformamide, triethylamine, dimethylsulfoxide, water or mixtures thereof) and, in the case of biphasic reaction conditions, optionally in the presence of a phase transfer catalyst;
(xv) for compounds of formula I in which X¹ represents —N(R⁹)-J-R¹⁰, J represents a single bond and R¹⁰ represents a C₁₋₈ alkyl group, reduction of a corresponding compound of formula I, in which J represents —C(O)— and R¹⁰ represents H or a C₁₋₇ alkyl group, in the presence of a suitable reducing agent. A suitable reducing agent may be an appropriate reagent that reduces the amide group to the amine group in the presence of other functional groups (for example an ester or a carboxylic acid). Suitable reducing agents include borane and other reagents known to the skilled person;
(xvi) for compounds of formula I in which X¹ represents halo, reaction of a compound of formula I wherein X¹ represents H, with a reagent or mixture of reagents known to be a source of halide atoms. For example, for bromide atoms, N-bromosuccinimide, bromine or 1,2-dibromotetrachloroethane may be employed, for iodide atoms, iodine, diiodoethane, diiodotetrachloroethane or a mixture of NaI or KI and N-chlorosuccinimide may be employed, for chloride atoms, N-chlorosuccinimide may be employed and for fluoride atoms, 1-(chloromethyl)-4-fluoro-1,4-diazoniabicyclo[2.2.2]octane bis(tetrafluoroborate), 1-fluoropyridinium triflate, xenon difluoride, CF₃OF or perchloryl fluoride may be employed. This reaction may be carried out in a suitable solvent (e.g. acetone, benzene or dioxane) under conditions known to the skilled person;
(xvii) for compounds of formula I in which R⁶ is other than H, reaction of a compound of formula XVII,

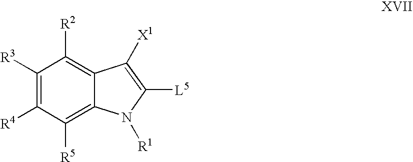

wherein L⁵ represents an appropriate alkali metal group (e.g. sodium, potassium or, especially, lithium), a —Mg-halide, a zinc-based group or a suitable leaving group such as halo or —B(OH)₂, or a protected derivative thereof (the skilled person will appreciate that the compound of formula XVII in which L⁵ represents an alkali metal (e.g. lithium), a Mg-halide or a zinc-based group may be prepared from a corresponding compound of formula XVII in which L⁵ represents halo, for example under conditions such as those hereinbefore described in respect of preparation of compounds of formula I (process step (x) above)), and X¹, R¹, R², R³, R⁴ and R⁵ are as hereinbefore defined, with a compound of formula XVIII,

L⁶C(O)OR^{6a} XVIII

wherein R^{6a} represents R⁶ provided that it does not represent H, and L⁶ represents a suitable leaving group such as halo (especially chloro or bromo) under conditions known to those skilled in the art. The skilled person will appreciate that L⁵ and L⁶ (when they both represent leaving groups) will be mutually compatible in a similar manner to the L¹ and L² groups described hereinbefore in process step (ii) above;
(xviii) for compounds of formula I in which R⁶ is H, reaction of a compound of formula XVII in which L⁵ represents either:
(I) an alkali metal (for example, such as one defined in respect of process step (xvii) above); or
(II) —Mg-halide,
   with carbon dioxide, followed by acidification under standard conditions known to those skilled in the art, for example, in the presence of aqueous hydrochloric acid;
   (xix) reaction of a corresponding compound of formula XVII in which L⁵ is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound of formula XIX,

R⁶OH XIX

wherein R⁶ is as hereinbefore defined, and an appropriate catalyst system (e.g. a palladium catalyst such as one described hereinbefore in respect of process step (ii)) under conditions known to those skilled in the art;
(xx) for compounds of formula I in which R⁶ represents H, hydrolysis of a corresponding compound of formula I in which R⁶ does not represent H under standard conditions;
(xxi) for compounds of formula I in which R⁶ does not represent H:
(A) esterification of a corresponding compound of formula I in which R⁶ represents H; or
(B) trans-esterification of a corresponding compound of formula I in which R⁶ does not represent H (and does not represent the same value of R⁶ as the compound of formula I to be prepared),
   under standard conditions in the presence of the appropriate alcohol of formula XIX as hereinbefore defined but in which R⁶ represents R^{6a};
   (xxii) for compounds of formula I in which X¹ represents -Q-X² in which Q represents —O—, reaction of a compound of formula XX,

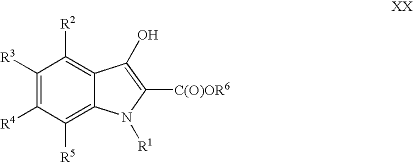

wherein R¹, R², R³, R⁴, R⁵ and R⁶ are as hereinbefore defined, with a compound of formula XXI,

X²L⁷ XXI

wherein L⁷ represents a suitable leaving group, such as a halo or sulfonate group and X² is as hereinbefore defined, for example in the presence of a base or under reaction conditions such as those described hereinbefore in respect of process (xiii) above;
(xxiii) for compounds of formula I in which X¹ represents —N(R⁹)-J-R¹⁰, reaction of a compound of formula XX as hereinbefore defined, with a compound of formula VI in which X^{1b} represents —N(R⁹)-J-R¹⁰ and R⁹, R¹⁰ and J are as hereinbefore defined, for example under reaction conditions known to those skilled in the art (such as those described in Journal of Medicinal Chemistry 1996, Vol. 39, 4044 (e.g. in the presence of MgCl₂));
(xxiv) for compounds of formula I in which X¹ represents -Q-X², Q represents a single bond and X² represents C₁₋₈ alkyl or heterocycloalkyl substituted α to the indole ring by a G¹ substituent in which G¹ represents -A¹-R^{11a}, A¹ represents —OA⁵-, A⁵ represents a single bond and R^{11a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, under conditions known to those skilled in the art, for example optionally in the presence of an acid, such as a protic acid or an appropriate Lewis acid. Such substitutions are described in inter alia Bioorg. Med. Chem. Lett., 14, 4741-4745 (2004) and Tetrahedron Lett. 34, 1529 (1993);
(xxv) for compounds of formula I in which X¹ represents -Q-X², Q represents a single bond and X² represents C₂₋₈ alkyl substituted (e.g. α to the indole ring) by a G¹ substituent in which G¹ represents -A¹-R^{11a}, A¹ represents —OA⁵-, A⁵ represents a single bond and R^{11a} represents H, reaction of a corresponding compound of formula I in which X² represents C₁₋₇ alkyl substituted (e.g. α to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl, under conditions known to those skilled in the art;
(xxvi) for compounds of formula I in which X¹ represents -Q-X², Q represents a single bond, and X² represents C₁₋₈ alkyl or heterocycloalkyl, both of which are unsubstituted in the position α to the indole ring, reduction of a corresponding compound of formula I in which X² represents C₁₋₈ alkyl substituted α to the indole ring by a G¹ substituent in which G¹ represents -A¹-R^{11a}, A¹ represents —OA⁵-, A⁵ represents a single bond and R^{11a} represents H, in the presence of a suitable reducing agent such as a mixture of triethyl silane and a protic acid (e.g. CF₃COOH) or a Lewis acid (e.g. (CH₃)₃SiOS(O)₂CF₃) for example under conditions described in inter alia Bioorg. Med. Chem. Lett., 14, 4741-4745 (2004);
(xxvii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O under conditions known to those skilled in the art, for example employing NaBH₄ in the presence of an acid (e.g. CH₃COOH or CF₃COOH), Wolff-Kishner reduction conditions (i.e. by conversion of the carbonyl group to a hydrazone, followed by base induced elimination) or by conversion of the carbonyl to the thioacetal analogue (e.g. by reaction with a dithiane) followed by reduction with e.g. Raney nickel, all under reaction conditions known to those skilled in the art; or
(xxviii) for compounds of formula I in which one of the groups R², R³, R⁴ or R⁵ represents a heterocycloalkyl group linked to the benzenoid moiety of the indole ring by a nitrogen atom, reaction of a compound of formula X as hereinbefore defined with a compound of formula XXIA,

(R^{2y-5y})H XXIA

wherein (R^{2y-5y}) represents R²⁻⁵ as hereinbefore defined provided that the appropriate R², R³, R⁴ or R⁵ substituent represents a heterocycloalkyl group in which the hydrogen atom of the compound of formula XXIA is attached to a nitrogen atom of that group, for example under similar conditions to those described hereinbefore in respect of processes (i) and/or (ii) above.

Compounds of formula II may be prepared by:
(a) reaction of a compound of formula XXII,

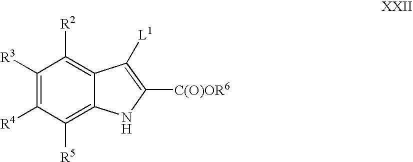

wherein L¹, R², R³, R⁴, R⁵ and R⁶ are as hereinbefore defined, with, for compounds of formula II in which X¹ represents:
   (1) Q-X² and Q represents a single bond or —C(O)—, a compound of formula V as hereinbefore defined; or
   (2) —N(R⁹)-J-R¹⁰ or -Q-X², in which Q represents —O— or —S—, a compound of formula VI as hereinbefore defined;
for example under reaction conditions similar to those described hereinbefore in respect of preparation of compounds of formula I (processes (ii) and (iv), respectively) above;
(b) for compounds of formula II in which X¹ represents -Q-X² and Q represents —C(O)—, reaction of a corresponding compound of formula II in which X¹ represents H with a compound of formula V in which Q^{a} represents —C(O)— and L² represents a suitable leaving group, for example under conditions such as those described in respect of preparation of compounds of formula I (process (iii)) above.
(c) for compounds of formula II in which X¹ represents -Q-X² and Q represents —S—, reaction of a compound of formula II in which X¹ represents H with a compound of formula VI in which X^{1b} represents -Q-X² and Q represents —S—, for example under conditions such as those described hereinbefore in respect of preparation of compounds of formula I (process (v)) above;
(d) for compounds of formula II in which X¹ represent Q-X² and Q represents —S(O)— or —S(O)₂—, oxidation a corresponding compound of formula II in which Q represent —S—;
(e) for compounds of formula II in which X¹ represents -Q-X², X² represents C₁₋₈ alkyl substituted by G¹, G¹ represents -A¹-R^{11a}, A¹ represents —N(R^{12a})A⁴- and A⁴ is a single bond (provided that Q represents a single bond when X² represents substituted C₁ alkyl), reaction of a compound of formula XXIII,

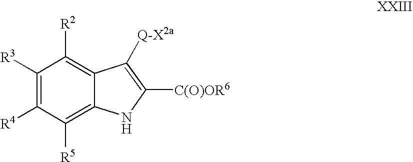

wherein Q, X^{2a}, R², R³, R⁴, R⁵ and R⁶ are as hereinbefore defined by reductive amination in the presence of a compound of formula VIII as hereinbefore defined;
(ea) for compounds of formula II in which X¹ represents -Q-X², Q represents a single bond, X² represents methyl substituted by G¹, G¹ represents -A¹-R^{11a}, A¹ represents —N(R^{12a})A⁴-, A⁴ is a single bond and R^{11a} and R^{12a} are preferably methyl, reaction of a corresponding compound of formula II in which X¹ represents H, with a mixture of formaldehyde (or equivalent reagent) and a compound of formula VIII as hereinbefore defined, for example under reaction conditions similar to those described hereinbefore in respect of preparation of compounds of formula I (process (viia)) above;
(f) for compounds of formula II in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula XXII in which L¹ represents halo (e.g. iodo) with a compound of formula IXC as hereinbefore defined, or reaction of a compound of formula XXIII in which Q represents a single bond and X^{2a} represents —CHO with a compound of formula IXB or a compound of formula IXC as hereinbefore defined, for example under reaction conditions similar to those described hereinbefore in respect of preparation of compounds of formula I (process (viii)) above;
(g) for compounds of formula II in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction (e.g. hydrogenation) of a corresponding compound of formula II in which X² represents optionally substituted C₂₋₈ alkenyl, cycloalkenyl, heterocycloalkenyl, C₂₋₈ alkynyl, cycloalkynyl or heterocycloalkynyl (as appropriate);
(h) for compounds of formula II in which one or more of R² to R⁵ represents -D-E and D represents —C(O)—, —C(R⁷)(R⁸)—, C₂₋₈ alkylene or —S(O)₂—, or optionally substituted cycloalkyl or heterocycloalkyl, reaction of a compound of formula XXIV,

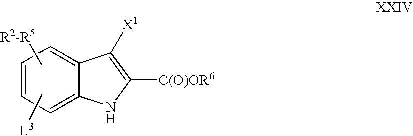

wherein X¹, L³, R²-R⁵ and R⁶ are as hereinbefore defined with a compound of formula XI or XIA (as appropriate) as hereinbefore defined, for example under reaction conditions similar to those described hereinbefore in respect of preparation of compounds of formula I (process (x)) above;
(i) for compounds of formula II in which when one of R² to R⁵ represents -D-E- and D represents —S—, —O— or C₂₋₄ alkynylene in which the triple bond is adjacent to E, reaction of a compound of formula XXIV as hereinbefore defined in which L³ represents L² as hereinbefore defined (for example —B(OH)₂) with a compound of formula XII as hereinbefore defined, for example under reaction conditions similar to those described hereinbefore in respect of preparation of compounds of formula I (process (xi)) above;
   (j) for compounds of formula II in which when one of R² to R⁵ represents -D-E- and D represents —S(O)— or —S(O)₂—, oxidation of a corresponding compound of formula II in which D represents —S—;
   (k) for compounds of formula II in which when one of R² to R⁵ represents -D-E- and D represents —O— or —S—, reaction of a compound of formula XXV,

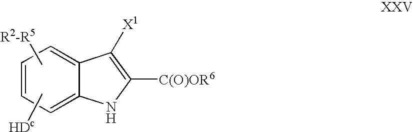

wherein D^{c}, X¹, R²-R⁵ and R⁶ are as hereinbefore defined, with a compound of formula XIV as hereinbefore defined;
(l) for compounds of formula II in which X¹ represents —N(R⁹)-J-R¹⁰, reaction of a compound of formula XXVI,

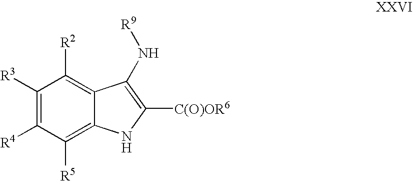

wherein R², R³, R⁴, R⁵, R⁶ and R⁹ are as hereinbefore defined with a compound of formula XVI as hereinbefore defined, for example under reaction conditions similar to those described hereinbefore in respect of preparation of compounds of formula I (process (xiv)) above;
(m) for compounds of formula II in which X¹ represents —N(R⁹)-J-R¹⁰, J represents a single bond and R¹⁰ represents a C₁₋₈ alkyl group, reduction of a corresponding compound of formula II, in which J represents —C(O)— and R¹⁰ represents H or a C₁₋₇ alkyl group, for example under reaction conditions similar to those described hereinbefore in respect of preparation of compounds of formula I (process (xv)) above;
(n) for compounds of formula II in which X¹ represents halo, reaction of a compound of formula II wherein X¹ represents H, with a reagent or mixture of reagents known to be a source of halide atoms, for example under reaction conditions similar to those described hereinbefore in respect of preparation of compounds of formula I (process (xvi)) above;
(o) for compounds of formula II in which R⁶ is other than H, reaction of a compound of formula XXVII,

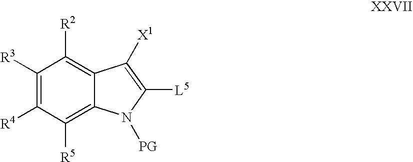

wherein PG represents a suitable protecting group (such as —S(O)₂Ph, —C(O)O⁻, —C(O)OtBu or —C(O)N(Et)₂) and L⁵, X¹, R², R³, R⁴ and R⁵ are as hereinbefore defined, with a compound of formula XVIII as hereinbefore defined, or a protected derivative thereof, for example under similar coupling conditions to those described hereinbefore in respect of process (xvii) above, followed by deprotection of the resultant compound under standard conditions;
(p) for compounds of formula II in which R⁶ is H, reaction of a compound of formula XXVII in which L⁵ represents an alkali metal, or —Mg-halide, with carbon dioxide, followed by acidification;
(q) reaction of a corresponding compound of formula XXVII in which L⁵ is a suitable leaving group known to those skilled in the art (such as a halo (e.g. bromo or iodo) group) with CO (or a reagent that is a suitable source of CO), in the presence of a compound of formula XIX as hereinbefore defined;
(r) for compounds of formula II in which R⁶ represents H, hydrolysis of a corresponding compound of formula II in which R⁶ does not represent H;
(s) for compounds of formula II in which R⁶ does not represent H:
(A) esterification of a corresponding compound of formula II in which R⁶ represents H; or
(B) trans-esterification of a corresponding compound of formula II in which R⁶ does not represent H (and does not represent the same value of R⁶ as the compound of formula II to be prepared);
(t) for compounds of formula II in which X¹ represents -Q-X² in which Q represents —O—, reaction of a compound of formula XXVIII,

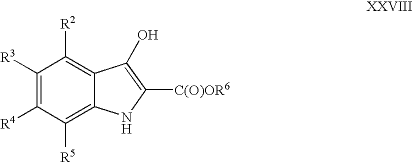

wherein R², R³, R⁴, R⁵ and R⁶ are as hereinbefore defined, with a compound of formula XXI as hereinbefore defined, for example under reaction conditions similar to those described hereinbefore in respect of preparation of compounds of formula I (process (xxii)) above;
(u) for compounds of formula II in which X¹ represents —N(R⁹)-J-R¹⁰, reaction of a compound of formula XXVIII as hereinbefore defined, with a compound of formula VI in which X^{1b} represents —N(R⁹)-J-R¹⁰ and R⁹, R¹⁰ and J are as hereinbefore defined, for example under conditions similar to those described hereinbefore in respect of preparation of compounds of formula I (process (xxiii)) above;
(v) for compounds of formula II in which X¹ represents -Q-X², Q represents a single bond and X² represents C₁₋₈ alkyl or heterocycloalkyl substituted α to the indole ring by a G¹ substituent in which G¹ represents -A¹-R^{11a}, A¹ represents —OA⁵-, A⁵ represents a single bond and R^{11a} represents H, reaction of a corresponding compound of formula II in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, for example under reaction conditions similar to those described hereinbefore in respect of preparation of compounds of formula I (process (xxiv)) above;
(w) for compounds of formula II in which X¹ represents -Q-X², Q represents a single bond and X² represents C₂₋₈ alkyl substituted (e.g. α to the indole ring) by a G¹ substituent in which G¹ represents -A¹-R^{11a}, A¹ represents —OA⁵-, A⁵ represents a single bond and R^{11a} represents H, reaction of a corresponding compound of formula II in which X² represents C₁₋₇ alkyl substituted (e.g. α to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl, under conditions known to those skilled in the art;
(x) for compounds of formula II in which X¹ represents -Q-X², Q represents a single bond, and X² represents C₁₋₈ alkyl or heterocycloalkyl, both of which are unsubstituted in the position α to the indole ring, reduction of a corresponding compound of formula II in which X² represents C₁₋₈ alkyl substituted α to the indole ring by a G¹ substituent in which G¹ represents -A¹-R^{11a}, A¹ represents —OA⁵-, A⁵ represents a single bond and R^{11a} represents H, for example under reaction conditions similar to those described hereinbefore in respect of preparation of compounds of formula I (process (xxvi)) above;
(y) for compounds of formula II in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula II in which X² represents C₁₋₈ alkyl or heterocycloalkyl, which groups are substituted by one or more Z¹ groups in which Z¹ represents ═O, for example under reaction conditions similar to those described hereinbefore in respect of preparation of compounds of formula I (process (xxvii)) above; or
(z) for compounds of formula II in which one of the groups R², R³, R⁴ or R⁵ represents a heterocycloalkyl group linked to the benzenoid moiety of the indole ring by a nitrogen atom, reaction of a compound of formula XXIV as hereinbefore defined (or a protected derivative thereof) with a compound of formula XXIA as hereinbefore defined, for example under similar conditions to those hereinbefore described in respect of preparation of compounds of formula I (process (xxviii)) above.

Compounds of formula IV may be prepared as follows:
(a) Reaction of a compound of formula XXII as hereinbefore defined with a compound of formula XXIX,

R¹L² XXIX
wherein R¹ and L² are as hereinbefore defined or a compound of formula III as hereinbefore defined, for example under reaction conditions similar to those described hereinbefore in respect of preparation of compounds of formula I (processes (ii) and (i), respectively) above; or
(b) for compounds of formula IV wherein L¹ represents a sulfonate group, reaction of a compound of formula XX with an appropriate reagent for the conversion of the hydroxyl group to the sulfonate group (e.g. tosyl chloride, mesyl chloride, triflic anhydride and the like) under conditions known to those skilled in the art.

Compounds of formula VII may be prepared by:
(a) for compounds of formula VII in which one of R² to R⁵ represents -D-E and D represents —C(O)—, —C(R⁷)(R⁸)—, C₂₋₈ alkylene or —S(O)₂—, or optionally substituted cycloalkyl or heterocycloalkyl, reaction of a compound of formula XXX,

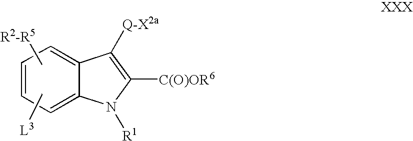

wherein Q, X^{2a}, L³, R¹, R²-R⁵ and R⁶ are as hereinbefore defined (L³ in particular may represent halo, such as bromo) with a compound of formula XI or XIA (as appropriate) as hereinbefore defined (in which L⁴ may in particular represent —B(OH)₂), for example under reaction conditions similar to those described hereinbefore in respect of preparation of compounds of formula I (process (x)) above;
(b) reaction of a compound of formula XXIII as hereinbefore defined with a compound of formula III as hereinbefore defined, for example under reaction conditions similar to those described hereinbefore in respect of preparation of compounds of formula I (process (i)) above); or
(c) for compounds of formula VII in which Q represents a single bond and X^{2a} represents —CHO, reaction of a corresponding compound of formula I in which X¹ represents H with a mixture of DMF and, for example, oxalyl chloride, phosgene or P(O)Cl₃ (or the like) in an appropriate solvent system (e.g. DMF or dichloromethane).

Compounds of formula X may be prepared by reaction of a compound of formula XXIV as hereinbefore defined, with a compound of formula III as hereinbefore defined, for example under reaction conditions similar to those described hereinbefore in respect of preparation of compounds of formula I (process (i)) above.

Compounds of formula X in which L³ represents L² may be prepared by reaction of a compound of formula X in which L³ represents L¹, with an appropriate reagent for the conversion of the L¹ group to the L² group. This conversion may be performed by methods known to those skilled in the art, for example, compounds of formula X, in which L³ is 4,4,5,5-tetramethyl-1,3,2-dioxaborolan-2-yl may be prepared by reaction of the reagent bis(pinacolato)diboron with a compound of formula X in which L³ represents L¹, for example under reaction conditions similar to those described hereinbefore in respect of preparation of compounds of formula I (process (ii)) above).

Compounds of formulae XV and XXVI may be prepared by reaction of a corresponding compound of formula IV, or XXII, respectively, with a compound of formula XXXI,

R⁹NH₂ XXXI

wherein R⁹ is as hereinbefore defined, for example under reaction conditions similar to those described hereinbefore in respect of preparation of compounds of formula I (process (ii)) above).

Compounds of formula XVII and XXVII in which L⁵ represents an appropriate alkali metal, such as lithium may be prepared by reaction of a compound of formula XXXII,

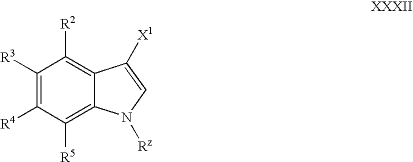

wherein R^{z} represents R¹ (in the case of a compound of formula XVII) or PG (in the case of a compound of formula XXVII), and PG, X¹, R¹, R², R³, R⁴ and R⁵ are as hereinbefore defined, with an appropriate base, such lithium diisopropylamide or BuLi under standard conditions. Compounds of formulae XVII and XXVII in which L⁵ represents —Mg-halide may be prepared from a corresponding compound of formula XVII or XXVII (as appropriate) in which L⁵ represents halo, for example under conditions such as those described hereinbefore in respect of process step (x). Compounds of formulae XVII and XXVII in which L⁵ represents, for example, a zinc-based group, or a halo or boronic acid group, may be prepared by reacting a corresponding compound of formula XVII or XXVII in which L⁵ represents an alkali metal with an appropriate reagent for introduction of the relevant group, for example by a metal exchange reaction (e.g. a Zn transmetallation), by reaction with a suitable reagent for the introduction of a halo group (for example, a reagent described hereinbefore in respect of preparation of compounds of formula I (process (xvi)) or, for the introduction of a boronic acid group, reaction with, for example, boronic acid or a protected derivative thereof (e.g. bis(pinacolato)diboron or triethyl borate) followed by (if necessary) deprotection under standard conditions.

Compounds of formula XXII may be prepared by standard techniques. For example, compounds of formula XXII in which one of R² to R⁵ represents an optionally substituted cycloalkyl or heterocycloalkyl group, or in which one of R² to R⁵ represents -D-E and D represents —C(O)—, —C(R⁷)(R⁸)—, C₂₋₄ alkylene or —S(O)₂—, may be prepared by reaction of a compound of formula XXXIII,

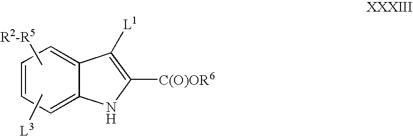

wherein L¹, L³, R²-R⁵ and R⁶ are as hereinbefore defined with a compound of formula XI (when one of R² to R⁵ represents -D-E and D represents —C(O)—, —C(R⁷)(R⁸)—, C₂₋₄ alkylene or —S(O)₂—) or XIA or XXIA (when one of R² to R⁵ represents optionally substituted cycloalkyl or heterocycloalkyl) as hereinbefore defined, for example under reaction conditions similar to those described hereinbefore in respect of preparation of compounds of formula I (process (x)) above.

Compounds of formulae XXIII and XXX, in which Q represents a single bond and X^{2a} represents —CHO, may be prepared from compounds of formulae II, or X, respectively, in which X¹ represents H, by reaction with a mixture of DMF and, for example, oxalyl chloride, phosgene or P(O)Cl₃ (or the like) in an appropriate solvent system (e.g. DMF or dichloromethane) for example as described hereinbefore.

Compounds of formulae III, V, VI, VIII, IXA, IXB, IXC, XI, XIA, XII, XIII, XIV, XVI, XVIII, XIX, XX, XXI, XXIA, XXIV, XXV, XXVIII, XXIX, XXXI, XXXII and XXXIII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and 1. Fleming, Pergamon Press, 1991.

Indoles of formulae II, IV, VII, X, XIII, XV, XVII, XX, XXII, XXIII, XXIV, XXV, XXVI, XXVII, XXVIII, XXX, XXXII and XXXIII may also be prepared with reference to a standard heterocyclic chemistry textbook (e.g. “Heterocyclic Chemistry” by J. A. Joule, K. Mills and G. F. Smith, 3^{rd} edition, published by Chapman & Hall or “Comprehensive Heterocyclic Chemistry II” by A. R. Katritzky, C. W. Rees and E. F. V. Scriven, Pergamon Press, 1996) and/or made according to the following general procedures.

For example, compounds of formulae II, XXIV and XXV, in which X¹ represents H, —N(R⁹)-J-R¹⁰ or -Q-X², may be prepared by reaction of a compound of formula XXXIV,

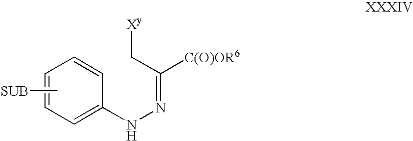

wherein SUB represents the substitution pattern that is present in the relevant compound to be formed (in this case, the compound of formula II, XXIV or XXV, respectively), X^{y} represents H, —N(R⁹)-J-R¹⁰ or -Q-X², and R⁶, R⁹, R¹⁰, J, X² and Q are as hereinbefore defined, under Fischer indole synthesis conditions known to the person skilled in the art.

Compounds of formulae II, XXIV and XXV in which X¹ represents H may be prepared by reaction of a compound of formula XXXV,

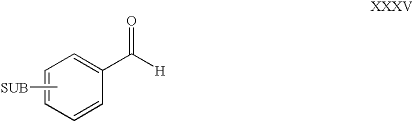

wherein SUB is as hereinbefore defined with a compound of formula XXXVI,

N₃CH₂C(O)OR⁶ XXXVI

wherein R⁶ is as hereinbefore defined, and preferably does not represent hydrogen, under conditions known to the person skilled in the art (i.e. conditions to induce a condensation reaction, followed by a thermally induced cyclisation).

Compounds of formulae XX and XXVIII may be prepared by reaction of a compound of formula XXXVII,

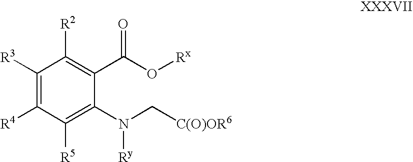

wherein R^{x} represents a C₁₋₆ alkyl group, R^{y} represents either R¹ (as required for the formation of compounds of formula XX), hydrogen (as required for the formation of compounds of formula XXVIII) or a nitrogen-protected derivative thereof, and R¹, R², R³, R⁴, R⁵ and R⁶ are as hereinbefore defined for example under cyclisation conditions known to those skilled in the art.

Compounds of formulae II, XXIV and XXV in which X¹ represents —NH₂, may be prepared by reaction of a compound of formula XXXVIII,

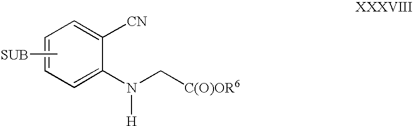

wherein SUB and R⁶ are as hereinbefore defined, for example under intramolecular cyclisation conditions known to those skilled in the art.

Compounds of formulae II and XXIV in which X¹ represents H, —N(R⁹)-J-R¹⁰ or -Q-X² in which Q represents a single bond or —C(O)—, may alternatively be prepared by reaction of a compound of formula XXXIX,

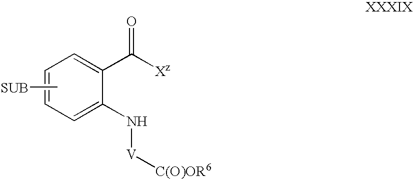

wherein V represents either —C(O)— or —CH₂—, X^{z} represents H, —N(R⁹)-J-R¹⁰ or -Q-X² in which Q represents a single bond or —C(O)— and SUB, R⁹, R¹⁰, J, X² and R⁶ are as hereinbefore defined. When V represents —C(O)—, the intramolecular cyclisation may be induced by a reducing agent such as TiCl₃/C₈K, TiCl₄/Zn or SmI₂ under conditions known to the skilled person, for example, at room temperature in the presence of a polar aprotic solvent (such as THF). When V represents —CH₂—, the reaction may be performed in the presence of base under intramolecular condensation reaction conditions known to the skilled person.

Compounds of formula XXXIV may be prepared by:
(a) reaction of a compound of formula XL,

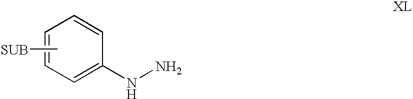

wherein SUB is as hereinbefore defined with a compound of formula XLI,

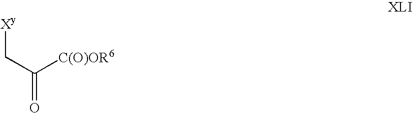

wherein X^{y} and R⁶ are as hereinbefore defined under condensation conditions known to the skilled person; or
(b) reaction of a compound of formula XLII,

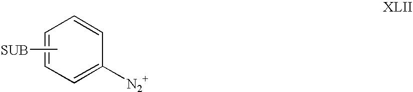

wherein SUB is as hereinbefore defined with a compound of formula XLIII,

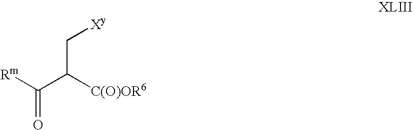

wherein R^{m} represents OH, O—C₁₋₆ alkyl or C₁₋₆ alkyl and X^{y} and R⁶ are as hereinbefore defined, for example under Japp-Klingemann conditions known to the skilled person.

Compounds of formula XXXIX may be prepared by reaction of a compound of XLIV,

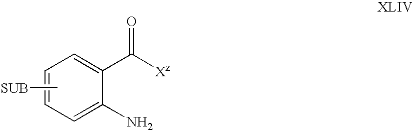

wherein SUB and X^{z} are as hereinbefore defined with a compound of formula XLV,

R⁶O(O)C—V—Cl XLV

wherein R⁶ and V are as hereinbefore defined, under standard coupling conditions.

Compounds of formulae XXXV, XXXVI, XXXVII, XXXVIII, XL, XLI, XLII, XLIII, XLIV and XLV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X¹, R¹, R², R³, R⁴, R⁵ and R⁶ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. For example, in cases where R⁶ does not initially represent hydrogen (so providing an ester functional group), the skilled person will appreciate that at any stage during the synthesis (e.g. the final step), the relevant substituent may be hydrolysed to form a carboxylic acid functional group (in which case R⁶ will be hydrogen). In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without provisos (b) and (d), for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which R⁶ is other than hydrogen) may possess no or minimal pharmacological activity as such, but may be administered parenterally or orally, and thereafter be metabolised in the body to form compounds of the invention that possess pharmacological activity as such (including, but not limited to, corresponding compounds of formula I, in which R⁶ represents hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention may inhibit the activity of leukotriene C₄ (LTC₄), for example as may be shown in a test such as that described in Eur. J. Biochem., 208, 725-734 (1992), and may thus be useful in the treatment of those conditions in which inhibition of LTC₄ is required. Compounds of the invention may also inhibit the activity of 5-lipoxygenase-activating protein (FLAP), for example as may be shown in a test such as that described in Mol. Pharmacol., 41, 873-879 (1992).

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (e.g. mPGES-1), LTC₄ and/or FLAP and/or a method of treatment of a disease in which inhibition of the activity of a member of the MAPEG family such as PGES (and particularly mPGES-1), LTC₄ and/or FLAP is desired and/or required (e.g. inflammation), which method comprises administration of a therapeutically effective amount of a compound of the invention, as hereinbefore defined but without the provisos, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without provisos (b) and (d), in admixture with a pharmaceutically acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined but without the provisos; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos, another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined but without the provisos, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay mPGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples, in which the following abbreviations may be employed:

BINAP 2,2′-bis(diphenylphosphino)-1,1′-binaphthalene
Boc tert-butoxycarbonyl
cy cyclohexyl
dba dibenzylideneacetone
DIBAL diisobutylaluminium hydride
DMAP 4,4-dimethylaminopyridine
DMF dimethylformamide
DMSO dimethylsulfoxide
DPEphos bis-(2-diphenylphosphinophenyl)ether
EtOAc ethyl acetate

### HPLC High Pressure Liquid Chromatography

MeCN acetonitrile
MS mass spectrum

### NBS N-bromosuccinimide

NMR nuclear magnetic resonance
rt room temperature
TFA trifluoroacetic acid
THF tetrahydrofuran
TLC thin layer chromatography
xantphos 9,9-dimethyl-4,5-bis(diphenylphosphino)xanthene

Starting materials and chemical reagents specified in the syntheses described below are commercially available from, e.g. Sigma-Aldrich Fine Chemicals.

### Example 1

### 5-(3-Isopropoxyphenoxy)-1-(4-isopropoxyphenyl)indole-2-carboxylic acid

### (a) 5-Benzyloxy-1-(4-isopropoxyphenyl)indole-2-carboxylic acid ethyl ester

An oven-dried vial (4 mL) was charged with K₃PO₄ (220 mg, 1.05 mmol), 5-benzyloxyindole-2-carboxylic acid ethyl ester (150 mg, 0.5 mmol) and flushed with argon. A solution of 4-isopropoxyphenyl bromide (150 mg, 0.7 mmol) in toluene (1.0 mL) was added, followed by a solution of CuI (22.9 mg, 0.12 mmol) and N,N′-dimethyl-1,2-diaminoethane (25.5 μL, 0.24 mmol) in toluene (1.2 mL). The mixture was heated at 110° C. for 20 h, cooled and filtered. The solids were washed with acetone and the combined filtrates concentrated and purified by chromatography affording the sub-title compound (163 mg, 75%).

### (b) 5-Hydroxy-1-(4-isopropoxyphenyl)indole-2-carboxylic acid ethyl ester

A mixture of 5-benzyloxy-1-(4-isopropoxyphenyl)indole-2-carboxylic acid ethyl ester (1.00 g, 2.3 mmol; see step (a) above), HCl (aq, conc, 0.42 mL) and EtOAc (15 mL) was hydrogenated at ambient temperature and pressure over 10% Pd on carbon (0.45 g) for 1.5 h. The mixture was filtered, the filtrate concentrated and the residue purified by chromatography to give the sub-title compound (0.70 g, 88%).

### (c) 5-(3-Isopropoxyphenoxy)-1-(4-isopropoxyphenyl)indole-2-carboxylic acid ethyl ester

Anhydrous CH₂Cl₂ (6 mL), Et₃N (164 μL, 1.18 mmol) and pyridine (93 mg, 1.18 mmol) were added to 5-hydroxy-1-(4-isopropoxyphenyl)indole-2-carboxylic acid ethyl ester (200 mg, 0.59 mmol; see step (b) above), Cu(OAc)₂ (107 mg, 0.59 mmol) and 3-isopropoxyphenylboronic acid (212 mg, 1.18 mmol). The mixture was stirred vigorously at rt for 24 h. After the reaction was complete (as judged by TLC), the mixture was filtered through Celite®, concentrated and purified by chromatography to give the sub-title compound (141 mg, 51%).

### (d) 5-(3-Isopropoxyphenoxy)-1-(4-isopropoxyphenyl)indole-2-carboxylic acid

A mixture of 5-(3-isopropoxyphenoxy)-1-(4-isopropoxyphenyl)indole-2-carboxylic acid ethyl ester (141 mg, 0.30 mmol; see step (c) above), dioxane (3.8 mL) and NaOH (aq, 2 M, 3.0 mL) was heated by microwave irradiation at 120° C. for 15 min. After cooling to rt the mixture was diluted with brine, neutralized to pH 2 by with HCl (aq, 1 M) and extracted with EtOAc. Concentration of the combined extracts and purification by chromatography gave the title compound (120 mg, 91%).

200 MHz ¹H-NMR (DMSO-d₆, ppm) δ 7.32-7.11 (4H, m) 7.07-6.90 (3H, m) 6.87-6.71 (2H, m) 6.59 (1H, d, J=8.8 Hz) 6.50-6.38 (2H, m) 6.36-6.28 (1H, m) 4.75-4.50 (2H, m) 1.33 (6H, d, J=6.2 Hz) 1.24 (6H, d, J=6.2 Hz).

### Example 2

### 5-(2-Isopropoxyphenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 2-isopropoxyphenylboronic acid.

200 MHz ¹H-NMR (DMSO-d₆, ppm) δ 7.28-7.16 (2H, m) 7.15-7.05 (4H, m) 7.04-6.86 (6H, m) 4.72-4.50 (2H, m) 1.31 (6H, d, J=6.0 Hz) 1.16 (6H, d, J=6.0 Hz).

### Example 3

### 5-(4-Isopropoxyphenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 4-isopropoxyphenylboronic acid.

200 MHz ¹H-NMR (DMSO-d₆, ppm) δ 7.20-7.08 (3H, m) 7.01-6.74 (10H, m) 4.70-4.42 (2H, m) 1.31 (6H, d, J=6.2 Hz) 1.24 (6H, d, J=6.2 Hz).

### Example 4

### 1-(4-Isopropoxyphenyl)-5-(3-trifluoromethoxyphenoxy)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3-trifluoromethoxyphenylboronic acid.

200 MHz ¹H-NMR (DMSO-d₆, ppm) δ 7.43 (1H, t, J=8.6 Hz) 7.29 (1H, d, J=2.2 Hz) 7.18 (1H, d, J=8.6 Hz) 7.06-6.92 (5H, m) 6.91-6.79 (3H, m) 6.74 (1H, s) 4.64 (1H, septet, J=6.0 Hz) 1.32 (6H, d, J=6.0 Hz)

### Example 5

### 5-(4-Trifluoromethoxyphenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 4-trifluoromethoxyphenylboronic acid.

200 MHz ¹H-NMR (DMSO-d₆, ppm) δ 13.1-12.4 (1H, br s) 7.45-7.39 (1H, m) 7.37-7.19 (5H, m) 7.08-6.96 (6H, m) 4.68 (1H, septet, J=6.0 Hz) 1.32 (6H, d, J=6.0 Hz).

### Example 6

### 5-(3,5-Dichlorophenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3,5-dichlorophenylboronic acid.

200 MHz ¹H-NMR (DMSO-d₆, ppm) δ 12.9-12.7 (1H, br s) 7.53-7.49 (1H, m) 7.33 (1H, s) 7.32-7.29 (2H, m) 7.25 (1H, s) 7.10-7.02 (3H, m) 7.01-6.93 (3H, m) 4.68 (1H, septet, J=6.0 Hz) 1.32 (6H, d, J=6.0 Hz).

### Example 7

### 5-(3-Chlorophenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3-chlorophenylboronic acid.

200 MHz ¹H-NMR (DMSO-d₆, ppm) δ 7.40-7.30 (2H, m) 7.28-7.08 (4H, m) 7.06-6.86 (6H, m) 4.68 (1H, septet, J=6.0 Hz) 1.32 (6H, d, J=6.0 Hz).

### Example 8

### 5-(4-tert-Butylphenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 4-tert-butylphenylboronic acid.

200 MHz ¹H-NMR (DMSO-d₆, ppm) δ 13.2-12.5 (1H, br s) 7.38-7.21 (6H, m) 7.03-6.98 (4H, m) 6.88 (2H, d, J=8.6 Hz) 4.68 (1H, septet, J=6.0 Hz) 1.34 (6H, d, J=6.0 Hz) 1.27 (9H, s).

### Example 9

### 1-(4-Isopropoxyphenyl)-5-(4-trifluoromethylphenoxy)indole-2-carboxylic acid hemihydrate

The title compound was prepared in accordance with steps (c) and (d) in Example 1 from 5-hydroxy-1-(4-isopropoxyphenyl)indol-2-carboxylic acid ethyl ester (see Example 1 step (b)) and 4-trifluoromethylphenylboronic acid.

200 MHz ¹H-NMR (DMSO-d₆, ppm) δ 13.2-12.6 (1H, br s) 7.72 (2H, d, J=8.7 Hz) 7.53-7.48 (1H, m) 7.35-7.24 (3H, m) 7.14-7.00 (6H, m) 4.69 (1H, septet, J=6.0 Hz) 1.35 (6H, d, J=6.0 Hz).

### Example 10

### 5-(4-Chlorophenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 4-chlorophenylboronic acid.

200 MHz ¹H-NMR (DMSO-d₆, ppm) δ 13.2-12.3 (1H, br s) 7.42-7.35 (3H, m) 7.30-7.22 (3H, m) 7.07-6.92 (6H, m) 4.67 (1H, septet, J=6.2 Hz) 1.33 (6H, d, J=6.2 Hz).

### Example 11

### 5-(3,4-Dichlorophenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3,4-dichlorophenylboronic acid.

200 MHz ¹H-NMR (DMSO-d₆, ppm) δ 7.55 (1H, d, J=8.4 Hz) 7.38-7.11 (4H, m) 7.06-6.69 (6H, m) 4.64 (1H, septet, J=6.2 Hz) 1.31 (6H, d, J=6.2 Hz).

### Example 12

### 1-(4-Isopropoxyphenyl)-5-(3-trifluoromethylphenoxy)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3-trifluoromethylphenylboronic acid.

200 MHz ¹H-NMR (DMSO-d₆, ppm) δ 7.64-7.52 (1H, m) 7.50-7.38 (2H, m) 7.35-7.20 (5H, m) 7.10-6.98 (4H, m) 4.68 (1H, septet, J=5.9 Hz) 1.34 (6H, d, J=5.9 Hz).

### Example 13

### 1-(4-Isopropoxyphenyl)-5-(quinolin-3-yloxy)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 3-quinolineboronic acid.

200 MHz ¹H-NMR (DMSO-d₆, ppm) δ 8.45-8.43 (1H, m) 8.00 (1H, d, J=8.2 Hz) 7.85 (1H, d, J=8.2 Hz) 7.71-7.46 (3H, m) 7.37 (1H, d, J=1.7 Hz) 7.19 (2H, d, J=8.6 Hz) 7.10-6.86 (4H, m) 6.78-6.72 (1H, m) 4.70-4.45 (1H, m) 1.32 (6H, d, J=6.0 Hz)

### Example 14

### 5-(4-Chloro-3-trifluoromethoxyphenoxy)-1-(4-isopropoxyphenyl)indole-2-carboxylic acid

### (a) 4-Bromo-1-chloro-2-trifluoromethoxybenzene

NaNO₂ (2.43 g, 0.035 mol) in water (10 mL) was added in portions over 30 min to 4-bromo-2-trifluoromethoxyaniline (9 g, 35 mmol) in a mixture of HCl (aq, conc, 25 mL) and water (25 mL) at (0-2° C.). The mixture was stirred at 0-2° C. for 15 min and CuCl (6 g, 61 mmol) in HCl (aq, conc, 10 mL) was added dropwise. After 10 min at rt, the mixture was heated at reflux for 15 min. Steam-distillation followed by extraction (CH₂Cl₂), drying (Na₂SO₄) of the distillate followed by concentration and distillation (bp 82-84° C. at 20 Torr) gave 3.86 g (40%) of the sub-title compound.

### (b) 4-Chloro-3-trifluoromethoxyphenylboronic acid

n-BuLi (2.5 M in hexanes; 6.25 mL, 12.5 mmol) was added dropwise to 4-bromo-1-chloro-2-trifluoromethoxybenzene (3.4 g, 12.3 mmol; see step (a) above) in anhydrous THF (50 mL) at −78° C. After 30 min, triethylborate (2.1 mL, 12.5 mmol) was added and the mixture was allowed to warm to rt and stirred at rt for 2 h. The mixture was poured into water (100 mL), acidified to pH 4 with HCl (aq, 1 M) and extracted with EtOAc (3×50 mL). The combined extracts were washed with brine, dried (Na₂SO₄) and concentrated. The residue was crystallised from petroleum ether to yield 2.07 g (70%) of the sub-title compound.

### (c) 5-(4-Chloro-3-trifluoromethoxyphenoxy)-1-(4-isopropoxyphenyl)indole-2-carboxylic acid

The title compound was prepared in accordance with steps (c) and (d) in Example 1 from 5-hydroxy-1-(4-isopropoxyphenyl)indol-2-carboxylic acid ethyl ester (see Example 1 step (b)) and 4-chloro-3-trifluoromethoxyphenylboronic acid (see step (b) above).

200 MHz ¹H-NMR (DMSO-d₆, ppm) δ 7.65-7.61 (1H, m) 7.51 (1H, s) 7.35 (1H, s) 7.30-7.25 (2H, m) 7.19 (1H, m) 7.08-6.94 (5H, m) 4.68 (1H, septet, J=5.8 Hz) 1.32 (6H, d, J=5.8 Hz).

### Example 15

### 5-(3-Chlorophenoxy)-1-(4-methyl-3-nitrophenyl)indole-2-carboxylic acid

### (a) 5-Benzyloxy-1-(4-methyl-3-nitrophenyl indole-2-carboxylic acid ethyl ester

4-Bromo-2-nitrotoluene (130 mg, 0.6 mmol) in toluene (1.0 mL) and CuI (95.2 mg, 0.5 mmol) and N,N′-dimethyl-1,2-diaminoethane (106 μL, 1.0 mmol) in toluene (1.0 mL) was added to 5-benzyloxyindole-2-carboxylic acid ethyl ester (150 mg, 0.5 mmol) and K₃PO₄ (220 mg, 1.05 mmol). The mixture was heated at 100-110° C. for 17 h, cooled and filtered. The solids were washed with acetone and the combined filtrates were concentrated and purified by chromatography to give the sub-title compound (177 mg, 66%).

### (b) 5-Hydroxy-1-(4-methyl-3-nitrophenyl)indole-2-carboxylic acid ethyl ester

A mixture of 5-benzyloxy-1-(4-methyl-3-nitrophenyl)indole-2-carboxylic acid ethyl ester (0.58 g, 1.34 mmol; see step (a) above), HCl (aq, conc, 0.23 mL) and EtOAc (30 mL) was hydrogenated at ambient temperature and pressure over 10% Pd—C (0.23 g) for 1.5 h. The mixture was filtered, concentrated and purified by chromatography to give the sub-title compound (0.136 g, 30%).

### (c) 5-(3-Chlorophenoxy)-1-(4-methyl-3-nitrophenyl)indole-2-carboxylic acid ethyl ester

Anhydrous CH₂Cl₂ (7 mL), Et₃N (160 μL, 1.18 mmol) and pyridine (96 μL, 1.18 mmol) were added to 5-hydroxy-1-(4-methyl-3-nitrophenyl)indole-2-carboxylic acid ethyl ester (200 mg, 0.59 mmol; see step (b) above), Cu(OAc)₂ (119 mg, 0.59 mmol) and 3-chlorophenylboronic acid (180 mg, 1.18 mmol). The mixture was stirred vigorously at rt for 48 h, filtered through Celite®, concentrated and purified by chromatography to give the sub-title compound (120 mg, 46%).

### (d) 5-(3-Chlorophenoxy)-1-(4-methyl-3-nitrophenyl)indole-2-carboxylic acid

NaOH (aq, 1 M, 6.0 mL) was added to 5-(3-chlorophenoxy)-1-(4-methyl-3-nitrophenyl)indole-2-carboxylic acid ethyl ester (141 mg, 0.30 mmol, see step (c) above) in acetone (3.0 mL). The mixture was stirred at rt for 5 h and acidified with HCl (aq, conc) to pH 2. The mixture was filtered, concentrated and purified by chromatography. Recrystallisation from MeOH/H₂O to afford the title compound (58 mg, 53%).

200 MHz ¹H-NMR (CDCl₃+DMSO-d₆, ppm) δ 8.02-7.97 (1H, m) 7.65-7.53 (2H, m) 7.42-7.37 (2H, m) 7.33-7.21 (1H, m) 7.14 (1H, d, J=9.1 Hz) 7.08-6.98 (2H, m) 6.93-6.83 (2H, m) 2.69 (3H, s).

### Example 16

### 5-(4-Chlorophenoxy)-1-(4-methyl-3-nitrophenyl)indole-2-carboxylic acid

The title compound was prepared in accordance with steps (c) and (d) in Example 15 from 5-hydroxy-1-(4-methyl-3-nitrophenyl)indol-2-carboxylic acid ethyl ester (see Example 15 step (b)) and 4-chlorophenylboronic acid.

200 MHz ¹H-NMR (CDCl₃, ppm) δ 8.04-8.00 (1H, m) 7.50 (3H, s) 7.35-7.23 (3H, m, overlapped with CHCl₃) 7.07 (2H, d) 6.96-6.86 (2H, m) 2.73 (3H, s).

### Example 17

### 5-(3,4-Dichlorophenoxy)-1-(4-methyl-3-nitrophenyl)indole-2-carboxylic acid

The title compound was prepared in accordance with steps (c) and (d) in Example 15 from 5-hydroxy-1-(4-methyl-3-nitrophenyl)indol-2-carboxylic acid ethyl ester (see Example 15 step (b)) and 3,4-dichlorophenylboronic acid.

200 MHz ¹H-NMR (CDCl₃, ppm) δ 8.02 (1H, t) 7.53-7.49 (3H, m) 7.38-7.32 (2H, m) 7.09-7.05 (2H, m) 7.03 (1H, d, J=2.8 Hz) 6.83 (1H, dd, J=8.9, 2.8 Hz) 2.73 (3H, s).

### Example 18

### 5-(3-Trifluoromethylphenoxy)-1-(4-methyl-3-nitrophenyl)indole-2-carboxylic acid

The title compound was prepared in accordance with steps (c) and (d) in Example 15 from 5-hydroxy-1-(4-methyl-3-nitrophenyl)indol-2-carboxylic acid ethyl ester (see Example 15 step (b)) and 3-trifluoromethylphenylboronic acid.

200 MHz ¹H-NMR (CDCl₃, ppm) δ 8.03 (1H, t) 7.54-7.45 (3H, m) 7.43-7.36 (2H, m) 7.34-7.26 (1H, m) 7.22-7.06 (4H, m) 2.73 (3H, s).

### Example 19

### 5-(3-Trifluoromethoxyphenoxy)-1-(4-methyl-3-nitrophenyl)indole-2-carboxylic acid

The title compound was prepared in accordance with steps (c) and (d) in Example 15 from 5-hydroxy-1-(4-methyl-3-nitrophenyl)indol-2-carboxylic acid ethyl ester (see Example 15 step (b)) and 3-trifluoromethoxyphenylboronic acid.

200 MHz ¹H-NMR (CDCl₃, ppm) δ 8.05-8.01 (1H, m) 7.57-7.47 (3H, m) 7.41-7.26 (2H, m) 7.09 (2H, d, J=1.2 Hz) 6.96-6.79 (3H, m) 2.73 (3H, s).

### Example 20

### 5-(3,5-Dichlorophenoxy)-1-(4-methyl-3-nitrophenyl)indole-2-carboxylic acid

The title compound was prepared in accordance with steps (c) and (d) in Example 15 from 5-hydroxy-1-(4-methyl-3-nitrophenyl)indol-2-carboxylic acid ethyl ester (see Example 15 step (b)) and 3,5-dichlorophenylboronic acid.

200 MHz ¹H-NMR (CDCl₃, ppm) δ 8.05-8.01 (1H, m) 7.58-7.46 (3H, m) 7.40 (1H, dd, J=2.0, 0.8 Hz) 7.01-7.06 (3H, m) 6.83 (2H, d, J=1.9 Hz) 4.6-3.8 (1H, br s) 2.73 (3H, s).

### Example 21

### 5-(4-Cyclohexylphenoxy)-1-(4-cyclopropoxyphenyl)indole-2-carboxylic acid

### (a) 1-Bromo-4-(2-bromoethoxy)benzene

A mixture of 4-bromophenol (30 g, 173 mmol), dibromoethane (40 mL, 464 mmol), NaOH (11.0 g, 275 mmol) and water (430 mL) was heated at reflux for 11 h. The layers were separated and the organic phase was concentrated and distilled to afford the sub-title compound (40.1 g 83%).

### (b) 1-Bromo-4-vinyloxybenzene

KOt-Bu (14.0 g, 125 mmol) was added in portions over 10 min to 1-bromo-4-(2-bromoethoxy)benzene (19.9 g, 100 mmol see step (a) above) in THF (120 mL) at 0° C. After 16 h at rt, water (400 mL) was added and the mixture was extracted with petroleum ether (4×100 mL). The combined extracts were washed with brine, dried (Na₂SO₄), concentrated and distilled under vacuum to yield the sub-title compound (11.5 g, 58%).

### (c) 1-Bromo-4-cyclopropoxybenzene

Diethylzinc (15% in hexanes, 95.5 mL, 116 mmol) was added to a mixture of 1-bromo-4-vinyloxybenzene (11.5 g, 58 mmol), chloro-iodomethane (41 g, 232 mmol) and dichloroethane (180 mL) over 3 h at 0° C. After 30 min NH₄Cl (aq, sat, 200 mL) and of petroleum ether (300 mL) were added. The organic phase was collected and concentrated. The residue was dissolved in petroleum ether, filtered and concentrated to afford the sub-title compound (11.7 g, 94%).

### (d) 5-(4-Cyclohexylphenoxy)-1-(4-cyclopropoxyphenyl)indole-2-carboxylic acid

The title compound was prepared in accordance with steps (a) and (b) in Example 1 from 5-benzyloxyindole-2-carboxylic acid ethyl ester and 1-bromo-4-cyclopropoxybenzene (see step (c) followed by arylation with 4-cyclohexylbenzeneboronic acid in accordance with step (c) in Example 1 and hydrolysis (step (d) in Example 1).

200 MHz ¹H-NMR (CDCl₃, ppm) δ 12.8-12.7 (1H, br s) 7.34-7.24 (4H, m) 7.21-7.10 (4H, m) 7.02-6.97 (2H, m) 6.89-6.80 (2H, m) 3.69-3.84 (1H, m) 2.47-2.36 (1H, m) 1.83-1.60 (5H, m) 1.46-1.18 (5H, m) 0.88-0.64 (4H, m)

### Example 22

### 1-(4-Isopropoxyphenyl)-5-(5-trifluoromethylpyridin-2-yloxy)indole-2-carboxylic acid

### (a) 1-(4-Isopropoxyphenyl)-5-(5-trifluoromethylpyridin-1-yloxy)indole-2-carboxylic acid ethyl ester

A mixture of 5-hydroxy-1-(4-isopropoxyphenyl)indole-2-carboxylic acid ethyl ester (200 mg, 0.59 mmol, see Example 1), 2-chloro-5-trifluoromethylpyridine (118 mg, 0.65 mmol) and K₂CO₃ (620 mg, 4.48 mmol) in DMF (8 mL) was heated at 70° C. for 4 h. The mixture was poured into water and extracted with EtOAc. The combined extracts were concentrated and purified by chromatography to afford the sub-title product (220 mg, 77%).

### (b) 1-(4-Isopropoxyphenyl)-5-(5-trifluoromethylpyridin-2-yloxy)indole-2-carboxylic acid

The title compound was prepared in accordance with Example 15 step (d) from 1-(4-isopropoxyphenyl)-5-(5-trifluoromethylpyridin-2-yloxy)indole-2-carboxylic acid ethyl ester (see step (a) above).

200 MHz ¹H-NMR (DMSO-d₆, ppm) δ 11.3-8.5 (1H, br s) 8.43 (1H, s) 7.88 (1H, dd, J=8.5, 1.7 Hz) 7.51-7.43 (2H, m) 7.24-7.14 (2H, m) 7.13-7.02 (2H, m) 7.02-6.91 (3H, m) 4.62 (1H, septet, J=6.2 Hz) 1.40 (6H, d, J=6.2 Hz).

### Example 23

### 1-(4-Isopropoxyphenyl)-6-(4-trifluoromethoxyphenoxy)indole-2-carboxylic acid

### (a) 2-Azido-3-(4-benzyloxyphenyl)acrylic acid ethyl ester

A solution of 4-benzyloxybenzaldehyde (10.00 g, 47.11 mmol) and azidoacetic acid ethyl ester (13.63 g, 118.5 mmol) in EtOH (130 mL) was added dropwise to a solution of NaOEt (8.06 g, 118.5 mmol) in EtOH (135 mL) at −10° C. The mixture was stirred at −10° C. for 2 h and poured into ice-cooled vigorously stirred NH₄Cl (aq, sat.). The mixture was extracted with EtOAc. The combined extracts were washed with brine, dried (Na₂SO₄), concentrated and purified by chromatography to afford the sub-title compound (10.73 g, 70%).

### (b) 6-Benzyloxyindole-2-carboxylic acid ethyl ester

A solution of 2-azido-3-(4-benzyloxyphenyl)-acrylic acid ethyl ester (10.7 g, 33.1 mmol; see step (a) above) in o-xylene (150 mL) was added dropwise to boiling o-xylene (150 mL). The heating was continued for 10 min and the solution was allowed to cool to rt and kept in a freezer (−18° C.) for 16 h. The precipitate was collected, washed with petroleum ether and dried to afford the sub-title compound (7.72 g, 83%).

### (c) 6-Benzyloxy-1-(4-isopropoxyphenyl)indole-2-carboxylic acid ethyl ester

An oven-dried vial was charged with K₃PO₄ (755 mg, 3.56 mmol), 6-benzyloxyindole-2-carboxylic acid ethyl ester (500 mg, 1.69 mmol, see step (b) above), CuI (32 mg, 0.17 mmol) and flushed with argon. A solution of 4-isopropoxyphenyl bromide (728 mg, 3.38 mmol) in toluene (9.0 mL) was added, followed by N,N-dimethyl-1,2-diaminoethane (54 μL, 0.51 mmol). The mixture was heated at 110° C. for 24 h, cooled and filtered through Celite®. The filtrate was concentrated and the residue purified by chromatography to afford the sub-title compound (630 mg, 87%).

### (d) 6-Hydroxy-1-(4-isopropoxyphenyl)indole-2-carboxylic acid ethyl ester

A mixture of 6-benzyloxy-1-(4-isopropoxyphenyl)indole-2-carboxylic acid ethyl ester (3.12 g, 7.26 mmol; see step (c) above) in EtOAc (50 mL) was hydrogenated at ambient temperature and pressure over 10% Pd—C (1.60 g) for 4 h. The mixture was filtered, concentrated and purified by chromatography to give the sub-title compound (2.35 g, 95%).

### (e) 1-(4-Isopropoxy-phenyl)-6-(4-tri fluoromethoxyphenoxy)indole-2-carboxylic acid ethyl ester

The subtitle compound was prepared in accordance Example 1 step (c) from 6-hydroxy-1-(4-isopropoxyphenyl)indole-2-carboxylic acid ethyl ester (see step (d) above) and 4-trifluoromethoxyphenylboronic acid.

### (f) 1-(4-Isopropoxyphenyl)-6-(4-trifluoromethoxyphenoxy)indole-2-carboxylic acid

A mixture of 1-(4-isopropoxyphenyl)-6-(4-trifluoromethoxyphenoxy)indole-2-carboxylic acid ethyl ester (156 mg, 0.31 mmol; see step (e) above), NaOH (aq, 1 M, 1.5 mL) and MeCN (10 mL) was heated at reflux for 2 h. The mixture was acidified to pH 2 with HCl (aq, 1 M) and extracted with EtOAc. The combined extracts were concentrated and purified by chromatography to afford the title product (120 mg, 82%).

200 MHz ¹H-NMR (DMSO-d₆, ppm) δ 12.73 (1H, s) 7.79 (1H, d, J=8.7 Hz) 7.39 (1H, d, J=0.7 Hz) 7.37-7.30 (2H, m) 7.27-7.18 (2H, m) 7.09-6.94 (4H, m) 6.93 (1H, dd, J=8.7, 2.1 Hz) 6.61-6.56 (1H, m) 4.65 (1H, septet, J=6.0 Hz) 1.30 (6H, d, J=6.0 Hz)

### Example 24

### 1-(4-Isopropoxyphenyl)-6-(3-trifluoromethoxyphenoxy)indole-2-carboxylic acid

The title compound was prepared in accordance with Example 1 step (c) from 6-hydroxy-1-(4-isopropoxyphenyl)indole-2-carboxylic acid ethyl ester (see Example 23 step (d)) and 3-trifluoromethoxyphenylboronic acid in accordance with Example 23 steps (e) and (f).

200 MHz ¹H-NMR (DMSO-d₆, ppm) δ 12.7 (1H, br s) 7.81 (1H, d, J=8.8 Hz) 7.45 (1H, dd, J=7.8, 7.8 Hz) 7.39 (1H, d, J=0.8 Hz) 7.27-7.18 (2H, m) 7.12-7.03 (1H, m) 7.02-6.92 (5H, m) 6.63-6.60 (1H, m) 4.65 (1H, septet, J=6.0 Hz) 1.30 (6H, d, J=6.0 Hz).

### Example 25

### 6-(6-Chloropyridin-2-yloxy)-1-(4-isopropoxyphenyl)indole-2-carboxylic acid

### (a) 6-(6-Chloropyridin-2-yloxy)-1-(4-isopropoxyphenyl)indole-2-carboxylic acid ethyl ester

A mixture of 6-hydroxy-1-(4-isopropoxyphenyl)indole-2-carboxylic acid ethyl ester (200 mg, 0.59 mmol, see Example 23 step (d)), 2,6-dichloropyridine (442 mg, 1.5 mmol), K₂CO₃ (1.05 g, 7.60 mmol) and DMF (10 mL) was heated at 90° C. for 26 h. The mixture was poured into water and extracted with EtOAc. The combined extracts were concentrated and purified by chromatography to afford the sub-title product (416 mg, 92%).

### (b) 6-(6-Chloropyridin-2-yloxy)-1-(4-isopropoxyphenyl)indole-2-carboxylic acid

The title compound was prepared in accordance with Example 23 step (e) from 6-(6-chloropyridin-2-yloxy)-1-(4-isopropoxyphenyl)indole-2-carboxylic acid ethyl ester (see step (a) above).

200 MHz ¹H-NMR (DMSO-d₆, ppm) δ 12.75 (1H, s) 7.87 (1H, d, J=7.9 Hz) 7.80 (1H, d, J=8.4 Hz) 7.41 (1H, s) 7.30-7.22 (2H, m) 7.21 (1H, d, J=7.7 Hz) 7.06-6.91 (4H, m) 6.79-6.75 (1H, m) 4.66 (1H, septet, J=6.0 Hz) 1.31 (6H, d, J=6.0 Hz).

### Example 26

### 1-(4-Isopropoxyphenyl)-6-(5-trifluoromethylpyridin-2-yloxy)indole-2-carboxylic acid

The title compound was prepared in accordance with Example 22 step (a) from 6-hydroxy-1-(4-isopropoxyphenyl)indole-2-carboxylic acid ethyl ester (see Example 23 step (d)) and 2-chloro-5-trifluoromethylpyridine (see Example 23 steps (e) and (f)).

200 MHz ¹H-NMR (DMSO-d₆, ppm) δ 12.6-12.9 (1H, br s) 8.54-8.48 (1H, m) 8.18 (1H, dd, J=8.8, 2.6 Hz) 7.81 (1H, d, J=8.6 Hz) 7.41 (1H, s) 7.30-7.21 (2H, m) 7.17 (1H, d, J=8.8 Hz) 7.05-6.95 (3H, m) 6.79-6.76 (1H, m) 4.65 (1H, septet, J=6.0 Hz) 1.30 (6H, d, J=6.0 Hz).

### Example 27

### 6-(3,4-Dichlorophenoxy)-1-(4-isopropoxyphenyl)indole-2-carboxylic acid

The title compound was prepared in accordance with Example 1 step (c) from 6-hydroxy-1-(4-isopropoxyphenyl)indole-2-carboxylic acid ethyl ester (see Example 23 step (d)) and 3,4-dichlorophenylboronic acid (see Example 23 steps (e) and (f)).

200 MHz ¹H-NMR (DMSO-d₆, ppm) δ 12.75 (1H, s) 7.80 (1H, d, J=8.7 Hz) 7.56 (1H, d, J=8.9 Hz) 7.41-7.38 (1H, m) 7.30-7.19 (3H, m) 7.04-6.89 (4H, m) 6.66 (1H, d, J=1.7 Hz) 4.66 (1H, septet, J=6.0 Hz) 1.30 (6H, d; J=6.0 Hz).

### Example 28

### 3-Chloro-1-(4-isopropoxyphenyl)-5-(4-trifluoromethylphenoxy)indole-2-carboxylic acid

### (a) 5-Benzyloxy-1-(4-isopropoxyphenyl)indole-2-carboxylic acid ethyl ester

An oven-dried vial was charged with K₃PO₄ (2.9 g, 13.7 mmol), 5-benzyloxyindole-2-carboxylic acid ethyl ester (2.0 g, 6.77 mmol) and flushed with argon. A solution of 4-isopropoxyphenylbromide (1.75 g, 8.14 mmol) in toluene (7.0 mL) was added, followed by a solution of CuI (193 mg, 1.01 mmol) and N,N′-dimethyl-1,2-diaminoethane (216 μL, 2.03 mmol) in toluene (5.0 mL). The mixture was heated at 90° C. for 48 h, cooled, poured into NH₄Cl (aq, sat, 50 mL) and extracted with EtOAc (3×50 mL). The combined extracts were washed with brine, dried (Na₂SO₄), filtered through silica gel and concentrated. The solid residue was recrystallised from EtOAc/petroleum ether to afford 2.5 g (86%) of the sub-title compound.

### (b) 5-Hydroxy-1-(4-isopropoxyphenyl)indole-2-carboxylic acid ethyl ester

A solution of 5-benzyloxy-1-(4-isopropoxyphenyl)indole-2-carboxylic acid ethyl ester (2.0 g, 4.6 mmol; see step (a) above) in EtOAc (30 mL) and EtOH (30 mL) was hydrogenated at ambient temperature and pressure over 10% Pd—C (490 mg, 0.546 mmol) for 2 h. The mixture was filtered through silica gel and concentrated. The residue was crystallised from EtOAc/petroleum ether to give the sub-title compound (1.3 g, 83%).

### (c) 5-Acetoxy-1-(4-isopropoxyphenyl)indole-2-carboxylic acid ethyl ester

Acetyl chloride (0.85 mL, 11.9 mmol) was added to 5-hydroxy-1-(4-isopropoxyphenyl)indole-2-carboxylic acid ethyl ester (2.7 g, 7.96 mmol; see step (b) above), DMAP (486 mg, 3.98 mmol) and Et₃N (3.4 mL, 23.9 mmol) in anhydrous CH₂Cl₂ (80 mL). After 12 h at rt, the mixture was poured into water (100 mL). HCl (aq, 1 M, 100 mL) was added and the mixture was extracted with EtOAc (3×50 mL). The combined extracts were washed with brine, dried (Na₂SO₄), filtered and concentrated to afford 2.9 g (95%) of the sub-title compound.

### (d) 5-Acetoxy-3-chloro-1-(4-isopropoxyphenyl)indole-2-carboxylic acid ethyl ester

SO₂Cl₂ (0.950 mL, 11.8 mmol) was added dropwise over 15 min to 5-acetoxy-1-(4-isopropoxyphenyl)indole-2-carboxylic acid ethyl ester (4.5 g, 11.8 mmol; see step (c) above) in anhydrous CH₂Cl₂ (200 mL) at 0° C. (dry ice bath). After 2 h at 0° C., the mixture was poured into NaHCO₃ (aq, sat, 200 mL) and extracted with EtOAc (3×100 mL). The combined extracts were washed with water, brine, dried (Na₂SO₄), filtered and concentrated to afford 4.0 g (82%) of the sub-title compound.

### (e) 3-Chloro-5-hydroxy-1-(4-isopropoxyphenyl)indole-2-carboxylic acid ethyl ester

5-Acetoxy-3-chloro-1-(4-isopropoxyphenyl)indole-2-carboxylic acid ethyl ester (1.41 g, 3.39 mmol; see step (d) above) was dissolved in MeOH saturated with ammonia (75 mL). The mixture was kept at 5° C. for 20 h and concentrated. The residue was dissolved in CH₂Cl₂ and filtered through silica gel and concentrated to afford 1.16 g (91%) of the sub-title compound.

### (f) 3-Chloro-1-(4-isopropoxyphenyl)-5-(4-trifluoromethylphenoxy)indole-2-carboxylic acid ethyl ester

Anhydrous CH₂Cl₂ (60 mL), triethylamine (380 μL, 2.68 mmol) and pyridine (220 mL, 2.68 mmol) were added to 3-chloro-5-hydroxy-1-(4-isopropoxyphenyl)-indole-2-carboxylic acid ethyl ester (500 mg, 1.34 mmol; see step (e) above), Cu(OAc)₂ (487 mg, 2.68 mmol) and 4-trifluoromethoxyphenyl boronic acid (509 mg, 2.68 mmol). The mixture was stirred vigorously at rt for 24 h, filtered through Celite®, concentrated and purified by chromatography to afford the sub-title compound (465 mg, 67%).

### (g) 3-Chloro-1-(4-isopropoxyphenyl)-5-(4-trifluoromethylphenoxy)indole-2-carboxylic acid

A mixture of 3-chloro-1-(4-isopropoxyphenyl)-5-(4-trifluoromethylphenoxy)-indole-2-carboxylic acid ethyl ester (155 mg, 0.30 mmol; see step (f) above), NaOH (aq, 2 M, 2.0 mL) and dioxane (4 mL) was heated at 120° C. After cooling to rt the mixture was diluted with brine, neutralized (pH 7) with HCl (aq, 1 M) and extracted with EtOAc. Concentration of the combined extracts and purification by chromatography gave the title product (120 mg, 91%).

200 MHz ¹H-NMR (DMSO-d₆, ppm) δ 7.32-7.11 (4H, m) 7.07-6.90 (3H, m) 6.87-6.71 (2H, m) 6.59 (1H, d, J=8.8 Hz) 6.50-6.38 (2H, m) 6.36-6.28 (1H, m) 4.75-4.50 (2H, m) 1.33 (6H, d, J=6.2 Hz) 1.24 (6H, d, J=6.2 Hz).

### Example 29

### 5-(4-tert-Butylphenoxy)-3-chloro-1-(4-isopropoxyphenyl)indole-2-carboxylic acid

### (a) 5-(4-tert-Butylphenoxy)-1-(4-isopropoxyphenyl)indole-2-carboxylic acid ethyl ester

The subtitle compound was prepared in accordance with Example 28 step (f) from 5-hydroxy-1-(4-isopropoxyphenyl)indole-2-carboxylic acid ethyl ester (see Example 28 step (b)) and 4-tert-butylphenylboronic acid.

### (b) 5-(4-tert-Butylphenoxy)-3-chloro-1-(4-isopropoxyphenyl)indole-2-carb-oxylic acid ethyl ester

A solution of SO₂Cl₂ (243 μL, 3.90 mmol) in anhydrous Et₂O (20 mL) was added over 10 min to 5-(4-tert-butylphenoxy)-1-(4-isopropoxyphenyl)indole-2-carboxylic acid ethyl ester (0.943 g, 2.0 mmol, see step (a), above) in anhydrous Et₂O (75 mL) at −9° C. The mixture was stirred at 0° C. for 24 h, washed with NaHCO₃ (aq, sat), water, and brine, dried (Na₂SO₄) and concentrated. The residue was treated with a small amount of petroleum ether and filtered, affording the sub-title compound (0.830 g, 82%).

### (c) 5-(4-tert-Butylphenoxy)-3-chloro-1-(4-isopropoxyphenyl)indole-2-carboxylic acid

The title compound was prepared in accordance with Example 28 step (g) from 5-(4-tert-butylphenoxy)-3-chloro-1-(4-isopropoxyphenyl)indole-2-carboxylic acid ethyl ester (see step (b) above).

200 MHz ¹H-NMR (CDCl₃, ppm) δ 10.50-8.0 (1H, br s) 7.38-7.28 (3H, m) 7.23-7.14 (2H, m) 7.09 (1H, dd, J=8.9, 2.0 Hz) 7.05-6.85 (5H, m) 4.61 (1H, septet, J=6.0 Hz) 1.39 (6H, d, J=6.0 Hz) 1.31 (9H, s).

### Example 30

### 5-(4-tert-Butylphenoxy)-3,4-dichloro-1-(4-isopropoxyphenyl)indole-2-carboxylic acid

### (a) 5-(4-tert-Butylphenoxy)-3,4-dichloro-1-(4-isopropoxyphenyl)indole-2-carboxylic acid ethyl ester

SO₂Cl₂ (80 μL, 0.98 mmol) in anhydrous CH₂Cl₂ (2 mL) was added to 5-(4-tert-butylphenoxy)-1-(4-isopropoxyphenyl)indole-2-carboxylic acid ethyl ester (200 mg, 0.47 mmol; see Example 29, step (a)) in anhydrous CH₂Cl₂ (3 mL) at rt. After 2 h the mixture was poured into NaHCO₃ (aq, sat) (caution! vigorous gas evolution). The phases were separated and the aqueous layer was extracted with CH₂Cl₂ (2×10 mL). The combined extracts were washed with Na₂S₂O₃ (aq, 10%), water and brine, dried (Na₂SO₄), concentrated and purified by chromatography to afford the sub-title compound (145 mg, 63%).

### (b) 5-(4-tert-Butylphenoxy)-3,4-dichloro-1-(4-isopropoxyphenyl)indole-2-carboxylic acid

The title compound was prepared in accordance with Example 28 step (g) from 5-(4-tert-butylphenoxy)-3,4-dichloro-1-(4-isopropoxyphenyl)indole-2-carboxylic acid ethyl ester (see step (a) above).

200 MHz ¹H-NMR (CDCl₃, ppm) δ 7.33-7.26 (2H, m) 7.22-7.13 (2H, m) 7.04 (1H, d, J=9.1 Hz) 7.00-6.93 (2H, m) 6.90 (1H, d, J=9.1 Hz) 6.86-6.78 (2H, m) 4.61 (1H, septet, J=6.1 Hz) 1.39 (6H, d, J=6.1 Hz) 1.29 (9H, s)

### Example 31

### 3-Chloro-1-(4-isopropoxyphenyl)-5-(3-trifluoromethoxyphenoxy)indole-2-carboxylic acid

The title compound was prepared from 1-(4-isopropoxyphenyl)-5-(5-trifluoromethylpyridin-2-yloxy)indole-2-carboxylic acid ethyl ester (see Example 22 step (a)) followed by chlorination (see Example 29 step (b) and hydrolysis (see Example 28 step (g)).

200 MHz ¹H-NMR (CDCl₃, ppm) δ 7.43-7.39 (1H, m) 7.36-7.27 (1H, m) 7.25-7.17 (2H, m) 7.11-7.07 (2H, m) 7.02-6.81 (5H, m) 4.61 (1H, septet, J=6.0 Hz) 1.40 (6H, d, J=6.1 Hz).

### Example 32

### 3-Chloro-1-(4-isopropoxyphenyl)-5-(4-trifluoromethoxyphenoxy)indole-2-carboxylic acid

The title compound was prepared from 5-hydroxy-1-(4-isopropoxyphenyl)indole-2-carboxylic acid ethyl ester (see Example 28 step (b)) and 4-trifluoromethoxyphenylboronic acid (see Example 28 step (f)) followed by chlorination (see Example 29 step (b)) and hydrolysis (see Example 28 step (g)).

200 MHz ¹H-NMR (CDCl₃, ppm) δ 7.37-7.33 (1H, m) 7.24-7.12 (4H, m) 7.08-7.04 (2H, m) 7.02-6.89 (4H, m) 4.59 (1H, septet, J=6.1 Hz) 1.38 (6H, d, J=6.1 Hz).

### Example 33

### 3-Chloro-5-(4-chloro-3-trifluoromethoxyphenoxy)-1-(4-isopropoxyphenyl)indole-2-carboxylic acid

The title compound was prepared in accordance with Example 28 step (f) from 3-chloro-5-hydroxy-1-(4-isopropoxyphenyl)indole-2-carboxylic acid ethyl ester (see Example 28 step (e)) and 4-chloro-3-trifluoromethoxyphenylboronic acid (see Example 14, step (b)) followed by hydrolysis (see Example 28 step (g)). 200 MHz ¹H-NMR (DMSO-d₆, ppm) δ 3.3 (1H, br s) 7.66 (1H, d, J=8.9 Hz) 7.40 (1H, dd, J=2.0, 0.7 Hz) 7.35-7.27 (2H, m) 7.27-7.23 (1H, m) 7.19 (1H, dd, J=9.0, 2.0 Hz) 7.12 (1H, dd, J=9.0, 0.7 Hz) 7.09-7.00 (2H, m) 7.01 (1H, dd, J=8.9, 2.8 Hz) 4.69 (1H, septet, J=6.0 Hz) 1.32 (6H, d, J=6.0 Hz).

### Example 34

### 3-Chloro-1-(4-isopropoxyphenyl)-5-(4-isopropoxy-3-trifluoromethoxyphenoxy)-indole-2-carboxylic acid

### (a) 4-Bromo-2-trifluoromethoxyphenol

Bromine (1.0 M in CH₂Cl₂, 45 mmol, 45 mL) was added dropwise to 2-trifluoromethoxyphenol (7.40 g, 41.5 mmol) in CH₂Cl₂ (100 mL) at −78° C. The mixture was allowed to warm to rt and was stirred for 48 hours. Na₂SO₃ (aq, sat, 100 mL) was added, and the mixture was stirred vigorously until the orange color disappeared. The mixture was diluted with CH₂Cl₂ (200 mL) and the organic layer collected, washed with brine, dried (Na₂SO₄) and concentrated to afford 9.6 g (91%) of the sub-title product.

### (b) 4-Bromo-1-isopropoxy-2-trifluoromethoxybenzene

A mixture of 4-bromo-2-trifluoromethoxyphenol (9.6 g, 37.4 mmol), 2-bromo-propane (7.0 mL, 74.7 mmol) and NaOH (3.0 g, 74.7 mmol) in anhydrous DMF (25 mL) was heated at 70° C. for 2 h, poured into water (100 mL) and extracted with t-BuOMe (3×100 mL). The combined extracts were washed with brine, dried (Na₂SO₄), concentrated and distilled (bulb-to-bulb, 150° C., 9.8×10⁻² Torr) to yield 9.5 g (85%) of the sub-title compound.

### (c) 4-Isopropoxy-3-trifluoromethoxyphenylboronic acid

The sub-title compound was prepared in accordance with Example 14 step (b) from 4-bromo-1-isopropoxy-2-trifluoromethoxybenzene (see step (b) above).

### (d) 3-Chloro-1-(4-isopropoxyphenyl)-5-(4-isopropoxy-3-trifluoromethoxyphenoxy)indole-2-carboxylic acid

The title compound was prepared in accordance with Example 28 step (f) from 3-chloro-5-hydroxy-1-(4-isopropoxyphenyl)indole-2-carboxylic acid ethyl ester (see Example 28 step (e)) and 4-isopropoxy-3-trifluoromethoxyphenylboronic acid (see step (c) above) followed by hydrolysis (see Example 28 step (g)).

200 MHz ¹H-NMR (DMSO-d₆, ppm) δ 13.3 (1H, br s) 7.34-7.19 (4H, m) 7.17-7.04 (4H, m) 7.03-6.95 (2H, m) 4.68 (1H, septet, J=6.0 Hz) 4.62 (1H, septet, J=6.0 Hz) 1.32 (6H, d, J=6.0 Hz) 1.27 (6H, d, J=6.0 Hz).

### Example 35

### 3-Chloro-5-(2,2-difluorobenzo[1,3]dioxol-5-yloxy)-1-(4-isopropoxyphenyl)-indole-2-carboxylic acid

### (a) 2,2-Difluorobenzo[1,3]dioxole-5-boronic acid

The sub-title compound was prepared in accordance with Example 14 step (b) from 5-bromo-2,2-difluorobenzo[1,3]-dioxole.

### (b) 3-Chloro-5-(2,2-difluorobenzo[1,3]dioxol-5-yloxy)-1-(4-isopropoxyphenyl)-indole-2-carboxylic acid

The title compound was prepared in accordance with Example 28 step (f) from 3-chloro-5-hydroxy-1-(4-isopropoxyphenyl)indole-2-carboxylic acid ethyl ester (see Example 28 step (e)) and 2,2-difluorobenzo[1,3]dioxole-5-boronic acid (see step (a) above) followed by hydrolysis (see Example 28 step (g)).

200 MHz ¹H-NMR (DMSO-d₆, ppm) δ 13.5-13.2 (1H, br s) 7.39 (1H, d, J=8.4 Hz) 7.34-7.23 (4H, m) 7.14 (1H, dd, J=9.0, 2.2 Hz) 7.11-7.09 (1H, m) 7.08-6.99 (2H, m) 6.82 (1H, dd, J=8.8, 2.4 Hz) 4.69 (1H, septet, J=6.0 Hz) 1.32 (6H, d, J=6.0 Hz).

### Example 36

### 3-Chloro-5-(3-fluoro-4-trifluoromethoxyphenoxy)-1-(4-isopropoxyphenyl)indole-2-carboxylic acid

### (a) 3-Fluoro-4-trifluoromethoxyphenylboronic acid

The sub-title compound was prepared in accordance with Example 14 step (b) from 4-bromo-2-fluoro-1-trifluoromethoxybenzene.

### (b) 3-Chloro-5-(3-fluoro-4-trifluoromethoxyphenoxy)-1-(4-isopropoxyphenyl)-indole-2-carboxylic acid

The title compound was prepared in accordance with Example 28 step (f) from 3-chloro-5-hydroxy-1-(4-isopropoxyphenyl)indole-2-carboxylic acid ethyl ester (see Example 28 step (e)) and 3-fluoro-4-trifluoromethoxyphenylboronic acid (see step (a) above) followed by hydrolysis (see Example 28 step (g)).

200 MHz ¹H-NMR (DMSO-d₆, ppm) δ 13.39 (1H, s) 7.61-7.49 (1H, m) 7.41 (1H, dd, J=1.6, 0.6 Hz) 7.36-7.26 (2H, m) 7.19 (1H, dd, J=9.0, 2.2 Hz) 7.06-6.99 (4H, m) 6.89-6.81 (1H, m) 4.69 (1H, septet, J=6.0 Hz) 1.32 (6H, d, J=6.0 Hz).

### Example 37

### 1-(4-Acetylaminophenyl)-3-chloro-5-(4-trifluoromethylphenoxy)indole-2-carboxylic acid

### (a) 1-(4-Acetylaminophenyl)-5-benzyloxy-3-chloroindole-2-carboxylic acid ethyl ester

The sub-title compound was prepared in accordance with Example 28, step (a) from 5-benzyloxyindole-2-carboxylic acid ethyl ester and (4-acetylamino)-phenylboronic acid, followed by chlorination (see Example 29, step (b)).

### (b) 1-(4-Acetylaminophenyl)-3-chloro-5-(4-trifluoromethylphenoxy)indole-2-carboxylic acid ethyl ester

The sub-title compound was prepared in accordance with Example 23, step (d) from 1-(4-acetylaminophenyl)-5-benzyloxy-3-chloroindole-2-carboxylic acid ethyl ester, followed by O-arylation (see Example 1, step (c)).

### (c) 1-(4-Acetylaminophenyl)-3-chloro-5-(4-trifluoromethylphenoxy)indole-2-carboxylic acid

The title compound was prepared in accordance with Example 28, step (g) from 1-(4-acetylaminophenyl)-3-chloro-5-(4-trifluoromethylphenoxy)indole-2-carboxylic acid ethyl ester (see step (b) above).

200 MHz ¹H-NMR (DMSO-d₆, ppm) δ 10.14 (1H, s) 7.80-7.63 (4H, m) 7.40-6.96 (7H, m) 2.08 (3H, s)

### Example 38

### 3-Chloro-1-(4-isopropoxyphenyl)-5-(5-trifluoromethylpyridin-2-yloxy)indole-2-carboxylic acid

The title compound was prepared from 5-hydroxy-1-(4-isopropoxyphenyl)indole-2-carboxylic acid ethyl ester (see Example 28 step (b)) and 2-chloro-5-trifluoro-methylpyridine (see Example 22 step (a)) followed by chlorination (see Example 29 step (b)) and hydrolysis (see Example 28 step (g)).

200 MHz ¹H-NMR (CDCl₃, ppm) δ 10.7-8.7 (1H, br s) 8.42 (1H, m) 7.90 (1H, dd, J=8.7, 2.3 Hz) 7.55-7.50 (1H, m) 7.23-7.14 (2H, m) 7.13-7.08 (2H, m) 7.05 (1H, d, J=8.7 Hz) 7.01-6.90 (2H, m) 4.61 (1H, septet, J=6.1 Hz) 1.40 (6H, d, J=6.0 Hz).

### Example 39

### 3-Chloro-1-(4-cyclopentyloxyphenyl)-5-(4-trifluoromethoxybenzoyl)indole-2-carboxylic acid

### (a) 5-Bromo-3-chloroindole-2-carboxylic acid ethyl ester

A mixture of 5-bromo-3-chloroindole-2-carboxylic acid ethyl ester (10.0 g, 37.3 mmol), SO₂Cl₂ (4.5 mL, 55.5 mmol) and benzene (250 mL) was heated at reflux for 2 h. Concentration to ca. 120 mL, cooling to rt and filtration afforded the sub-title compound (6.33 g, 56% yield).

### (b) 5-Bromo-3-chloro-1-(4-cyclopentyloxyphenyl)indole-2-carboxylic acid ethyl ester

Anhydrous CH₂Cl₂ (80 mL), Et₃N (2.7 mL, 19.8 mmol), pyridine (1.6 mL, 19.8 mmol) and 3 Å molecular sieves (ca. 3 g) were added to 5-bromo-3-chloroindole-2-carboxylic acid ethyl ester (3 g, 9.9 mmol; see step (a) above), Cu(OAc)₂ (3.6 g, 19.8 mmol), and 4-cyclopentyloxyphenylboronic acid (4.08 g, 19.8 mmol). The mixture was stirred vigorously at rt for 30 h and filtered through Celite®. The solids were washed with EtOAc and the combined filtrates were concentrated and purified by chromatography to afford the sub-title compound (3.4 g, 75%).

### (c) 3-Chloro-1-(4-cyclopentyloxyphenyl)-5-iodoindole-2-carboxylic acid ethyl ester

A mixture of 5-bromo-3-chloro-1-(4-cyclopentyloxyphenyl)indole-2-carboxylic acid ethyl ester (3.72 g, 8.04 mmol; see step (b)), CuI (0.152 g, 0.8 mmol), N,N′-dimethyl-1,2-diaminoethane (170 μL, 1.6 mmol), NaI (2.41 g, 16.0 mmol) and dioxane (15 mL) was heated at 110° C. for 72 h, cooled to rt, diluted with NH₄Cl (aq, sat), poured into water (200 mL) and extracted with EtOAc. The combined extracts were washed with water, brine, dried (Na₂SO₄), filtered through silica gel and concentrated to afford the sub-title compound (3.68 g, 72%).

### (d) 3-Chloro-1-(4-cyclopentyloxyphenyl)-5-(4-trifluoromethoxybenzoyl)indole-2-carboxylic acid ethyl ester

A solution of 3-chloro-1-(4-cyclopentyloxyphenyl)-5-iodoindole-2-carboxylic acid ethyl ester (255 mg, 0.5 mmol) was added dropwise to i-PrMgCl*LiCl in THF (1 M in THF, 0.5 mL, 0.5 mmol) at −40° C. under argon. After 15 min., 4-trifluoromethoxybenzoyl chloride (0.24 mL, 1.5 mmol) was added and the mixture was allowed to warm to rt. NH₄Cl (aq, sat, 2.0 mL) was added and the mixture was extracted with EtOAc. The combined extracts were washed with water, brine and dried (Na₂SO₄). Concentration and purification by chromatography afforded the sub-title compound (210 mg, 73%).

### (e) 3-Chloro-1-(4-cyclopentyloxyphenyl)-5-(4-trifluoromethoxybenzoyl)indole-2-carboxylic acid

A mixture of 3-chloro-1-(4-cyclopentyloxyphenyl)-5-(4-trifluoromethoxybenzoyl)indole-2-carboxylic acid ethyl ester (165 mg, 0.29 mmol; see step (d)), dioxane (2 mL) and NaOH (aq, 2 M, 1.0 mL, 2.0 mmol) was heated by microwave irradiation at 120° C. for 15 min. After cooling, a few drops of water were added, and the pH was adjusted to ca 2 by addition of HCl (aq, 2 M). The white precipitate was filtered off and recrystallised from EtOAc/petroleum ether to yield 156 mg (99%) of the title compound.

200 MHz ¹H-NMR (DMSO-d₆, ppm) δ 8.03-8.02 (1H, m) 7.92-7.85 (2H, m) 7.79 (1H, dd, J=8.8, 1.7 Hz) 7.57-7.53 (2H, m) 7.63-7.29 (2H, m) 7.19 (1H, d, J=9.0 Hz) 7.07-6.99 (2H, m) 4.92-4.83 (1H, m) 2.01-1.54 (8H, m).

### Example 40

### 3-Chloro-5-(4-chlorobenzoyl)-1-(4-cyclopentyloxyphenyl)indole-2-carboxylic acid

The title compound was prepared in accordance with Example 39, step (d) from 3-chloro-1-(4-cyclopentyloxyphenyl)-5-iodoindole-2-carboxylic acid ethyl ester (see Example 39, step (c)) and 4-chlorobenzoyl chloride, followed by hydrolysis (see Example 39, step (e)).

200 MHz ¹H-NMR (DMSO-d₆, ppm) δ 13.6-13.4 (1H, br s) 8.02-8.01 (1H, m) 7.80-7.74 (3H, m) 7.67-7.61 (2H, m) 7.36-7.29 (2H, m) 7.19 (1H, d, J=8.8 Hz) 7.07-7.00 (2H, m) 4.92-4.83 (1H, m) 2.02-1.54 (8H, m).

### Example 41

### 3-Chloro-1-(4-cyclopentyloxyphenyl)-5-(3-isopropoxybenzoyl)indole-2-carboxylic acid

The title compound was prepared in accordance with Example 39, step (d) from 3-chloro-1-(4-cyclopentyloxyphenyl)-5-iodoindole-2-carboxylic acid ethyl ester (see Example 39, step (c)) and 3-isopropoxybenzoyl chloride, followed by hydrolysis (see Example 39, step (e)).

200 MHz ¹H-NMR (DMSO-d₆, ppm) δ 13.6-13.4 (1H, br s) 8.03-8.02 (1H, m) 7.79 (1H, dd, J=8.8, 1.6 Hz) 7.50-7.41 (1H, m) 7.36-7.29 (2H, m) 7.27-7.16 (4H, m) 7.07-7.00 (2H, m) 4.91-4.84 (1H, m) 4.66 (1H, septet, J=6.0 Hz) 2.03-1.55 (8H, m) 1.27 (6H, d, J=6.0 Hz

### Example 42

### 3-Chloro-5-(6-chloropyridine-3-carbonyl)-1-(4-cyclopentyloxyphenyl)indole-2-carboxylic acid

The title compound was prepared in accordance with Example 39, step (d) from 3-chloro-1-(4-cyclopentyloxyphenyl)-5-iodoindole-2-carboxylic acid ethyl ester (see Example 39, step (c)) and 6-chloronicotinoyl chloride, followed by hydrolysis (see Example 39, step (e)).

200 MHz ¹H-NMR (DMSO-d₆, ppm) δ 14.0-13.0 (1H, br s) 8.73 (1H, dd, J=2.4, 0.8 Hz) 8.18 (1H, dd, J=8.3, 2.4 Hz) 8.07 (1H, dd, J=1.6, 0.6 Hz) 7.82 (1H, dd, J=8.8, 1.6 Hz) 7.73 (1H, dd, J=8.3, 0.8 Hz) 7.37-7.29 (2H, m) 7.20 (1H, dd, J=8.8, 0.6 Hz) 7.07-6.99 (2H, m) 4.92-4.84 (1H, m) 2.02-1.56 (8H, m).

### Example 43

### 3-Chloro-1-(4-isopropoxyphenyl)-5-(3-trifluoromethylbenzyl)indole-2-carboxylic acid

### (a) 3-Chloro-5-iodo-1-(4-isopropoxyphenyl)indole-2-carboxylic acid ethyl ester

The sub-title compound was prepared in accordance with Example 39 step (b) from 5-bromo-3-chloroindole-2-carboxylic acid ethyl ester (see step (a) Example 39) and 4-isopropoxyphenylboronic acid followed by bromine-iodine exchange (see Example 39 step (c)).

### (b) 3-Chloro-5-(dihydroxyboryl)-1-(4-isopropoxyphenyl)indole-2-carboxylic acid ethyl ester

i-PrMgCl*LiCl (0.95 M in THF, 3.26 mL, 3.1 mmol) was added over 5 min to 3-chloro-5-iodo-1-(4-isopropoxyphenyl)indole-2-carboxylic acid ethyl ester (1.45 g, 3.0 mmol, see step (a) above) in THF (9 mL) at −40° C. After 15 min at −40° C., B(OEt)₃ (1.56 mL, 9.0 mmol) was added. The temperature was allowed to reach 0° C. over 2 h and HCl (aq, 2.5 M, 14.4 mL, 36 mmol) was added. After 1 h at 0° C., the mixture was diluted with brine (70 mL) and extracted with t-BuOMe (4×70 mL). The combined extracts were washed with brine (100 mL), dried (Na₂SO₄) and concentrated. The solid residue was treated several times with petroleum ether and filtered to give the sub-title compound (1.04 g, 86%)

### (c) 3-Chloro-1-(4-isopropoxyphenyl)-5-(3-trifluoromethylbenzyl)indole-2-carboxylic acid ethyl ester

A mixture of 3-chloro-5-(dihydroxyboryl)-1-(4-isopropoxyphenyl)indole-2-carboxylic acid ethyl ester (160 mg, 0.4 mmol; see step (b)), 3-trifluoromethylbenzylbromide (0.25 mL, 1.6 mmol) Pd(OA)₂ (4.5 mg, 0.02 mmol), triphenylphosphine (10.5 mg, 0.04 mmol), K₃PO₄ (2.41 g, 16.0 mmol) and toluene (2 mL) was heated at 120° C. for 24 h, cooled to rt, diluted with EtOAc, washed with water and brine, dried (Na₂SO₄), concentrated and purified by chromatography to afford the sub-title compound (109 mg, 53%).

### (d) 3-Chloro-1-(4-isopropoxyphenyl)-5-(3-trifluoromethylbenzyl)indole-2-carboxylic acid

The title compound was prepared from 3-chloro-1-(4-isopropoxyphenyl)-5-(3-trifluoromethylbenzyl)indole-2-carboxylic acid ethyl ester (see step (c) above) followed by hydrolysis (see Example 28 step (g)).

200 MHz ¹H-NMR (DMSO-d₆, ppm) δ 13.4-13.1 (1H, br s) 7.65-7.43 (5H, m) 7.28-7.16 (3H, m) 7.06-6.92 (3H, m) 4.64 (1H, septet, J=6.0 Hz) 4.05 (2H, s) 1.28 (6H, d, J=6.0 Hz)

### Example 44

### 3-Chloro-5-(3-chlorobenzyl)-1-(4-isopropoxyphenyl)indole-2-carboxylic acid

The title compound was prepared from 3-chloro-5-(dihydroxyboryl)-1-(4-isopropoxyphenyl)indole-2-carboxylic acid ethyl ester (see Example 43 step (b)) and 3-chlorobenzylchloride (see Example 43 step (c)) followed by hydrolysis (see Example 28 step (g)).

200 MHz ¹H-NMR (DMSO-d₆, ppm) δ 7.54 (1H, s) 7.76-7.14 (7H, m) 7.04-6.96 (3H, m) 4.64 (1H, septet, J=6.0 Hz) 4.12 (2H, s) 1.28 (6H, d, J=6.0 Hz).

### Example 45

### 5-(3-Chlorophenylsulfanyl)-1-(4-cyclopentyloxyphenyl)indole-2-carboxylic acid

### (a) 1-(4-Cyclopentyloxyphenyl)-5-iodoindole-2-carboxylic acid ethyl ester

The sub-title compound was prepared in accordance with Example 39 step (b) from 5-bromoindole-2-carboxylic acid ethyl ester and 4-cyclopentyloxy-phenylboronic acid followed by bromine-iodine exchange (see Example 39 step (c)).

### (b) 5-(3-Chlorophenylsulfanyl)-1-(4-cyclopentyloxyphenyl)indole-2-carboxylic acid ethyl ester

A mixture of 1-(4-cyclopentyloxyphenyl)-5-iodoindole-2-carboxylic acid ethyl ester (180 mg, 0.38 mmol; see step (a)), 3-chlorobenzenethiol (46 μL, 0.42 mmol) Pd₂(dba)₃ (10.4 mg, 0.011 mmol), DPEphos (12.2 mg, 0.023 mmol), KOt-Bu (47.1 g, 0.42 mmol) and toluene (3 mL) was heated at 100° C. for 2 h. The mixture was cooled to rt, diluted with EtOAc, filtered through Celite®, concentrated and purified by chromatography to afford the sub-title compound (140 mg, 75%).

### (c) 5-(3-Chlorophenylsulfanyl)-1-(4-cyclopentyloxyphenyl)indole-2-carboxylic acid

The title compound was prepared from 5-(3-chlorophenylsulfanyl)-1-(4-cyclopentyloxyphenyl)indole-2-carboxylic acid ethyl ester (see step (b) above) followed by hydrolysis (see Example 23 step (f).

200 MHz ¹H-NMR (DMSO-d₆, ppm) δ 13. 12.9-12.8 (1H, br s) 7.99 (1H, d, J=1.8 Hz) 7.42-7.16 (6H, m) 7.13-6.96 (5H, m) 4.93-4.80 (1H, m) 2.01-1.51 (8H, m).

### Example 46

### 3-Chloro-5-(4-chlorophenylsulfanyl)-1-(4-isopropoxyphenyl)indole-2-carboxylic acid

### (a) 3-Chloro-5-(4-chlorophenylsulfanyl)-1-(4-isopropoxyphenyl)indole-2-carboxylic acid ethyl ester

The sub-title compound was prepared from 3-chloro-5-iodo-1-(4-isopropoxyphenyl)indole-2-carboxylic acid ethyl ester (see Example 43 step (a)) and 4-chlorobenzenethiol (see Example 45 step (b)).

### (b) 3-Chloro-5-(4-chlorophenylsulfanyl)-1-(4-isopropoxyphenyl)indole-2-carboxylic acid

The title compound was prepared from 3-chloro-5-(4-chlorophenylsulfanyl)-1-(4-isopropoxyphenyl)indole-2-carboxylic acid ethyl ester (see step (a) above) followed by hydrolysis (see Example 23 step (f)).

200 MHz ¹H-NMR (DMSO-d₆, ppm) δ 13.6-13.4 (1H, br s) 7.80 (1H, d, J=1.4 Hz) 7.45-7.25 (5H, m) 7.24-7.15 (2H, m) 7.12 (1H, d, J=8.8 Hz) 7.08-7.00 (2H, m) 4.68 (1H, septet, J=6.0 Hz) 1.32 (6H, d, J=6.0 Hz).

### Example 47

### 3-Chloro-1-(4-isopropoxyphenyl)-5-(4-trifluoromethylphenylsulfanyl)indole-2-carboxylic acid

### (a) 3-Chloro-1-(4-isopropoxyphenyl)-5-(4-trifluoromethylphenylsulfanyl)indole-2-carboxylic acid ester

The sub-title compound was prepared from 3-chloro-5-iodo-1-(4-isopropoxyphenyl)indole-2-carboxylic acid ethyl ester (see Example 43 step (a)) and 4-trifluoromethylbenzenethiol (see Example 45 step (b)).

### (b) 3-Chloro-1-(4-isopropoxyphenyl)-5-(4-trifluoromethylphenylsulfanyl)indole-2-carboxylic acid

The title compound was prepared from 3-chloro-1-(4-isopropoxyphenyl)-5-(4-trifluoromethylphenylsulfanyl)indole-2-carboxylic acid ester (see step (a) above) followed by hydrolysis (see Example 23 step (f)).

200 MHz ¹H-NMR (DMSO-d₆, ppm) δ 13.8-13.2 (1H, br s) 7.88 (1H, d, J=1.5 Hz) 7.66-7.53 (2H, m) 7.43 (1H, dd, J=8.8, 1.5 Hz) 7.35-7.20 (4H, m) 7.20-7.11 (1H, m) 7.07-6.96 (2H, m) 4.67 (1H, septet, J=5.9 Hz) 1.30 (6H, d, J=5.9 Hz).

### Example 48

### 3-Chloro-5-(4-chlorobenzenesulfinyl)-1-(4-isopropoxyphenyl)indole-2-carboxylic acid

### (a) 3-Chloro-5-(4-chlorobenzenesulfinyl)-1-(4-isopropoxyphenyl)indole-2-carboxylic acid ethyl ester

A mixture of 3-chloro-5-(4-chlorophenylsulfanyl)-1-(4-isopropoxyphenyl)indole-2-carboxylic acid ethyl ester (150 mg, 0.3 mmol; see step (a) Example 46), Bu₄NIO₄ (143 mg, 0.33 mmol), 5,10,15,20-tetraphenyl-21H,23,H-porphine iron (III) chloride (4 mg, 0.006 mmol) and CH₂Cl₂ (2 mL) was stirred at rt for 3.5 h, diluted with CH₂Cl₂, filtered through silica gel, concentrated and purified by chromatography to afford the sub-title compound (87 mg, 56%).

### (b) 3-Chloro-5-(4-chlorobenzenesulfinyl)-1-(4-isopropoxyphenyl)indole-2-carboxylic acid

The title compound was prepared from 3-chloro-5-(4-chlorobenzenesulfinyl)-1-(4-isopropoxyphenyl)indole-2-carboxylic acid ethyl ester (see step (a) above) followed by hydrolysis (see Example 1, step (d)).

200 MHz ¹H-NMR (DMSO-d₆, ppm) δ 13.6 (1H, br s) 8.15 (1H, d, J=1.5 Hz) 7.81-7.70 (2H, m) 7.65-7.56 (2H, m) 7.54 (1H, dd, J=9.0 1.5 Hz) 7.33-7.23 (2H, m) 7.17 (1H, d, J=9.0 Hz) 7.07-6.96 (2H, m) 4.67 (1H, septet, J=6.0 Hz) 1.31 (6H, d, J=6.0 Hz).

### Example 49

### 3-Chloro-1-(4-isopropoxyphenyl)-5-(4-trifluoromethylbenzenesulfinyl)indole-2-carboxylic acid

The title compound was prepared from 3-chloro-1-(4-isopropoxyphenyl)-5-(4-trifluoromethylphenylsulfanyl)indole-2-carboxylic acid ester (see step (a) Example 47) by oxidation (see Example 48, step (a)) followed by hydrolysis (see Example 23 step (f)).

200 MHz ¹H-NMR (DMSO-d₆, ppm) δ 8.00-7.82 (4H, m) 7.61-7.50 (1H, m) 7.31-7.20 (2H, m) 7.20-7.11 (1H, m) 7.04-6.93 (2H, m) 4.65 (1H, septet, J=6.2 Hz) 1.28 (6H, d, J=6.2 Hz).

### Example 50

### 3-Chloro-5-(4-chlorobenzenesulfonyl)-1-(4-isopropoxyphenyl)indole-2-carboxylic acid

### (a) 3-Chloro-5-(4-chlorobenzenesulfonyl)-1-(4-isopropoxyphenyl)indole-2-carboxylic acid ethyl ester

A mixture of 3-chloro-5-(4-chlorophenylsulfanyl)-1-(4-isopropoxyphenyl)indole-2-carboxylic acid ethyl ester (200 mg, 0.4 mmol; see step (a) Example 46), Oxone® (1.23 g, 2.0 mmol), THF (3 mL) and water (4 mL) was stirred at rt for 3 d, diluted with water and extracted with EtOAc. The combined extracts were washed with water and brine, concentrated and purified by chromatography to afford the sub-title compound (165 mg, 77%).

### (b) 3-Chloro-5-(4-chlorobenzenesulfonyl)-1-(4-isopropoxyphenyl)indole-2-carboxylic acid

The title compound was prepared from 3-chloro-5-(4-chlorobenzenesulfonyl)-1-(4-isopropoxyphenyl)indole-2-carboxylic acid ethyl ester (see step (a) above) followed by ester hydrolysis (see Example 23, step (f)).

200 MHz ¹H-NMR (DMSO-d₆, ppm) δ 13.7-12.6 (1H, br s) 8.32-8.23 (1H, m) 8.03-7.93 (2H, m) 7.84-7.73 (1H, m) 7.71-7.60 (2H, m) 7.35-7.24 (2H, m) 7.20 (1H, d, J=8.8 Hz) 7.07-6.95 (2H, m) 4.66 (1H, septet, J=5.9 Hz) 1.29 (6H, d, J=5.9 Hz).

### Example 51

### 3-Chloro-1-(4-isopropoxyphenyl)-5-(4-trifluoromethylbenzenesulfonyl)indole-2-carboxylic acid

The title compound was prepared from 3-chloro-1-(4-isopropoxyphenyl)-5-(4-trifluoromethylphenylsulfanyl)indole-2-carboxylic acid ester (see step (a) Example 47) by oxidation (see Example 50, step (a)) followed by hydrolysis (see Example 23 step (f)).

200 MHz ¹H-NMR (DMSO-d₆, ppm) δ 3.8-13.15 (1H, br s) 8.35 (1H, d, J=1.5 Hz) 8.26-8.15 (2H, m) 8.01-7.91 (2H, m) 7.84 (1H, dd, J=8.8, 1.5 Hz) 7.34-7.26 (2H, m) 7.26-7.19 (1H, m) 7.07-6.96 (2H, m) 4.66 (1H, septet, J=5.9 Hz) 1.26 (6H, d, J=5.9 Hz)

### Example 52

### 1-(4-Isopropoxyphenyl)-3-methanesulfonylamino-5-(4-trifluoromethylphenoxy)-indole-2-carboxylic acid

### (a) 3-Bromo-1-(4-isopropoxyphenyl)-5-(4-trifluoromethylphenoxy)indole-2-carboxylic acid ethyl ester

A mixture of 1-(4-isopropoxyphenyl)-5-(4-trifluoromethylphenoxy)indole-2-carboxylic acid ethyl ester (452 mg, 0.93 mmol, see Example 9), NBS (360 mg, 2.00 mmol) and CCl₄ (10 mL) was heated at 80° C. for 14 h. The mixture was poured into Na₂S₂O₃ (aq, 10%) and extracted with CH₂Cl₂. The combined extracts were washed with water, dried (Na₂SO₄) and purified by chromatography to give 489 mg (93%) of the sub-title compound.

### (b) 1-(4-Isopropoxyphenyl)-3-methanesulfonylamino-5-(4-trifluoromethylphenoxy)indole-2-carboxylic acid ethyl ester

An oven-dried pressure tube was charged with 3-bromo-1-(4-isopropoxyphenyl)-5-(4-trifluoromethylphenoxy)indole-2-carboxylic acid ethyl ester (300 mg, 0.53 mmol, see step (a) above), methanesulfonamide (101 mg, 1.06 mmol), Cs₂CO₃ (209 mg, 0.80 mmol), Pd₂(dba)₃ and xantphos (47 mg, 0.08 mmol). The tube was flushed with argon and dioxane (5 mL) was added. The mixture was heated at 90° C. for 48 h, cooled and filtered through Celite®. The filtrate was concentrated and purified by chromatography affording the sub-title compound (260 mg, 85%).

### (c) 1-(4-Isopropoxyphenyl)-3-methanesulfonylamino-5-(4-trifluoromethylphenoxy)indole-2-carboxylic acid

The title compound was prepared in accordance with Example 23 step (f) from 1-(4-isopropoxyphenyl)-3-methanesulfonylamino-5-(4-trifluoromethylphenoxy)-indole-2-carboxylic acid ethyl ester (see step (b), above).

200 MHz ¹H-NMR (DMSO-d₆, ppm) δ 12.0-11.0 (1H, br s) 7.72-7.63 (2H, m) 7.61-7.57 (1H, m) 7.25-7.15 (2H, m) 7.12-7.03 (2H, m) 7.03-6.90 (4H, m) 4.66 (1H, septet, J=6.0 Hz) 2.85 (3H, s) 1.32 (6H, d, J=6.0 Hz).

### Example 53

### 1-(4-Isopropoxyphenyl)-3-[(pyridine-3-carbonyl)amino]-5-(4-trifluoromethylphenoxy)indole-2-carboxylic acid

### (a) 1-(4-Isopropoxyphenyl)-3-[(pyridine-3-carbonyl)amino]-5-(4-trifluoromethylphenoxy)indole-2-carboxylic acid ethyl ester

An oven-dried pressure tube was charged with 3-bromo-1-(4-isopropoxyphenyl)-5-(4-trifluoromethylphenoxy)indole-2-carboxylic acid ethyl ester (170 mg, 0.30 mmol, see Example 52 step (a)), nicotinamide (74 mg, 0.60 mmol), K₃PO₄ (135 mg, 0.64 mmol) and CuI (12 mg, 0.06 mmol). The tube was flushed with argon and dioxane (4 mL) followed by N,N′-dimethyl-1,2-diaminoethane (16 μL, 0.15 mmol) were added. The mixture was heated at 90° C. for 24 h, cooled, filtered through Celite®, concentrated and purified by chromatography to give the sub-title compound (82 mg, 45%).

### (b) 1-(4-Isopropoxyphenyl)-3-[(pyridine-3-carbonyl)amino]-5-(4-trifluoromethylphenoxy)indole-2-carboxylic acid

The title compound was prepared in accordance with Example 23 step (f) from 1-(4-isopropoxyphenyl)-3-[(pyridine-3-carbonyl)amino]-5-(4-trifluoromethylphenoxy)indole-2-carboxylic acid ethyl ester (see step (a), above).

200 MHz ¹H-NMR (DMSO-d₆, ppm) δ 13.5-12.5 (1H, br s) 10.60 (1H, s) 9.18-9.12 (1H, m) 8.76 (1H, dd, J=4.6, 1.2 Hz) 8.36-8.28 (1H, m) 7.72-7.64 (2H, m) 7.61-7.52 (2H, m) 7.33-7.25 (2H, m) 7.13-7.01 (6H, m) 4.68 (1H, septet, J=6.0 Hz) 1.31 (6H, d, J=6.0 Hz).

### Example 54

### 1-(4-Isopropoxyphenyl)-3-(2-oxopyrrolidin-1-yl)-5-(4-trifluoromethylphenoxy)-indole-2-carboxylic acid

The title compound was prepared in accordance with Example 53 step (a) from 3-bromo-1-(4-isopropoxyphenyl)-5-(4-trifluoromethylphenoxy)indole-2-carboxylic acid ethyl ester (see Example 52 step (a)) and pyrrolidin-2-one, followed by hydrolysis (see Example 23 step (f)).

200 MHz ¹H-NMR (DMSO-d₆, ppm) δ 13.3-13.0 (1H, br s) 7.71-7.67 (2H, m) 7.34-7.25 (3H, m) 7.12 (1H, d, J=9.0 Hz) 7.09-6.96 (5H, m) 4.65 (1H, septet, J=6.0 Hz) 3.81 (2H, t, J=6.0 Hz) 2.39 (2H, t, J=8.0 Hz) 2.18-2.01 (2H, m) 1.30 (6H, d, J=6.0 Hz).

### Example 55

### 3-(4-Dimethylaminobutyrylamino)-1-(4-isopropoxyphenyl)-5-(4-trifluoromethylphenoxy)indole-2-carboxylic acid

The title compound was prepared in accordance with Example 52 step (b) from 3-bromo-1-(4-isopropoxyphenyl)-5-(4-trifluoromethylphenoxy)indole-2-carboxylic acid ethyl ester (see Example 52 step (a)) and 4-dimethylaminobutyramide followed by hydrolysis (see Example 23 step (f)).

200 MHz ¹H-NMR (DMSO-d₆, ppm) δ 7.85-7.81 (1H, m) 7.73-7.64 (2H, m) 7.22-7.13 (2H, m) 7.10-7.01 (2H, m) 7.01-6.94 (4H, m) 4.65 (1H, septet, J=6.0 Hz) 2.92-2.81 (2H, m) 2.56 (6H, s) 2.53-2.39 (2H, m, overlapped with DMSO) 1.99-1.81 (2H, m) 1.31 (6H, d, J=6.0 Hz).

### Example 56

### 3-(2,2-Dimethylpropionylamino)-1-(4-isopropoxyphenyl)-5-(4-isopropoxy-3-trifluoromethoxyphenoxy)indole-2-carboxylic acid

### (a) 3-Bromo-1-(4-isopropoxyphenyl)-5-(4-isopropoxy-3-trifluoromethoxyphenoxy)indole-2-carboxylic acid ethyl ester

The sub-title compound was prepared in accordance with Example 1 step (c) from 5-hydroxy-1-(4-isopropoxyphenyl)indole-2-carboxylic acid ethyl ester (see Example 1 step (b)) and 4-isopropoxy-3-trifluoromethoxyphenyl boronic acid (Example 34 step (a-c)) followed by bromination (see Example 52 step (a)).

### (b) 3-(2,2-Dimethylpropionylamino)-1-(4-isopropoxyphenyl)-5-(4-isopropoxy-3-trifluoromethoxyphenoxy)indole-2-carboxylic acid

The sub-title compound was prepared in accordance with Example 52 step (b) from 3-bromo-1-(4-isopropoxyphenyl)-5-(4-isopropoxy-3-trifluoromethoxyphenoxy)indole-2-carboxylic acid ethyl ester (see step (a) above) and 2,2-dimethylpropionamide followed by hydrolysis (see Example 23 step (f)).

200 MHz ¹H-NMR (DMSO-d₆, ppm) δ 13.6-12.7 (1H, br s) 10.5-10.0 (1H, br s) 7.57 (1H, s) 7.26-7.16 (3H, m) 7.06-6.94 (5H, m) 6.89 (1H, dd, J=9.0, 2.8 Hz) 4.67 (1H, septet, J=6.0 Hz) 4.59 (1H, septet, J=6.0 Hz) 1.32 (6H, d, J=6.0 Hz) 1.25 (6H, d, J=6.0 Hz) 1.24 (9H, s).

### Example 57

### 3-(2,2-Dimethylpropionylamino)-1-(4-isopropoxyphenyl)-5-(4-trifluoromethoxyphenoxy)indole-2-carboxylic acid

### (a) 5-Benzyloxy-3-iodoindole-2-carboxylic acid ethyl ester

A solution of NaI (3.05 g, 20.33 mmol) in acetone (70 mL) was added dropwise to N-chlorosuccinimide (2.71 g, 20.33 mmol) in acetone (50 mL) protected from light. After 15 min, a solution of 5-benzyloxyindole-2-carboxylic acid ethyl ester (5.00 g, 16.93 mmol) in acetone (145 mL) was added dropwise, followed by stirring for 30 min at rt. The mixture was poured into Na₂S₂O₃ (aq, 10%, 250 mL) and extracted with EtOAc. The combined extracts were washed with NaHCO₃ (aq, sat), water and brine, dried (Na₂SO₄) and concentrated. The residue was crystallised from EtOH to give the sub-title compound (7.13 g, 87%).

### (b) 5-Benzyloxy-3-iodo-1-(4-isopropoxyphenyl)indole-2-carboxylic acid ethyl ester

Anhydrous CH₂Cl₂ (100 mL), Et₃N (3.34 mL, 23.74 mmol) and pyridine (1.94 mL, 23.74 mmol) were added to 5-benzyloxy-3-iodoindole-2-carboxylic acid ethyl ester (5.00 g, 11.87 mmol; see step (a) above), Cu(OAc)₂ (4.31 g, 23.74 mmol), 3 Å molecular sieves (ca. 8 g) and 4-isopropoxyphenylboronic acid (4.27 g, 23.74 mmol). The mixture was stirred vigorously at rt for 24 h and filtered through Celite®. The solids were washed with EtOAc and the combined filtrates concentrated and purified by chromatography to afford the sub-title compound (6.07 g, 92%).

### (c) 5-Benzyloxy-3-(2,2-dimethylpropionylamino)-1-(4-isopropoxyphenyl)indole-2-carboxylic acid ethyl ester

The subtitle product was prepared in accordance with Example 53 step (a) from 5-benzyloxy-3-iodo-1-(4-isopropoxyphenyl)indole-2-carboxylic acid ethyl ester (see step (b) above) and 2,2-dimethylpropionamide.

### (d) 3-(2,2-Dimethylpropionylamino)-5-hydroxy-1-(4-isopropoxyphenyl)indole-2-carboxylic acid ethyl ester

The subtitle product was prepared in accordance with Example 23 step (d) from 5-benzyloxy-3-(2,2-dimethylpropionylamino)-1-(4-isopropoxyphenyl)indole-2-carboxylic acid ethyl ester (see step (c) above).

### (e) 3-(2,2-Dimethylpropionylamino)-1-(4-isopropoxyphenyl)-5-(4-trifluoromethoxyphenoxy)indole-2-carboxylic acid

The title compound was prepared in accordance with Example 1 step (c) from 3-(2,2-dimethylpropionylamino)-5-hydroxy-1-(4-isopropoxyphenyl)indole-2-carboxylic acid ethyl ester (see step (d) above) and 4-trifluoromethoxyphenylboronic acid (see Example 23 step (f)).

200 MHz ¹H-NMR (DMSO-d₆, ppm) δ 13.5-12.5 (1H, br s) 9.76 (0.8H, s) 8.88 (0.2H, s) 7.65-7.44 (3H, m) 7.32-7.16 (2H, m) 7.16-6.95 (5H, m) 7.71-6.89 (1H, m) 4.68 (1H, septet, J=6.0 Hz) 1.43 (1.8H, s) 1.32 (6H, d, J=6.0 Hz) 1.26 (7.2H, s).

### Example 58

### 3-(2,2-Dimethylpropionylamino)-1-(4-isopropoxyphenyl)-5-(4-trifluoromethylphenoxy)indole-2-carboxylic acid

The title compound was prepared in accordance with Example 1 step (c) from 3-(2,2-dimethylpropionylamino)-5-hydroxy-1-(4-isopropoxyphenyl)indole-2-carboxylic acid ethyl ester (see Example 57 step (d)) and 4-trifluoromethylphenylboronic acid according to Example 23 step (f).

200 MHz ¹H-NMR (DMSO-d₆, ppm) δ 13.4-13.0 (1H, br s) 10.3-10.0 (1H, br s) 7.74-7.62 (3H, m) 7.29-7.17 (2H, m) 7.10-6.95 (6H, m) 4.67 (1H, septet, J=6.0 Hz) 1.32 (6H, d, J=6.0 Hz) 1.25 (9H, s).

### Example 59

### 5-(4-Chloro-3-trifluoromethoxyphenoxy)-3-(2,2-dimethylpropionylamino)-1-(4-isopropoxyphenyl)indole-2-carboxylic acid

The title compound was prepared in accordance with Example 1 step (c) from 3-(2,2-dimethylpropionylamino)-5-hydroxy-1-(4-isopropoxyphenyl)indole-2-carboxylic acid ethyl ester (see Example 57 step (d)) and 4-chloro-3-trifluoromethoxyphenylboronic acid (see Example 14, step (b)), followed by hydrolysis (see Example 23 step (f)).

200 MHz ¹H-NMR (DMSO-d₆, ppm) δ 9.64 (1H, s) 7.64 (1H, d, J=8.0 Hz) 7.57 (1H, d, J=1.8 Hz) 7.30-7.21 (2H, m) 7.21-7.12 (1H, m) 7.11-7.00 (4H, m) 6.93 (1H, dd, J=9.1, 2.9 Hz) 4.68 (1H, septet, J=6.0 Hz) 1.32 (6H, d, J=6.0 Hz) 1.26 (9H, s).

### Example 60

### 3-[(2,2-Dimethylpropionyl)methylamino]-1-(4-isopropoxyphenyl)-5-(4-trifluoromethylphenoxy)indole-2-carboxylic acid

### (a) 5-Benzyloxy-3-[(2,2-dimethylpropionyl)methylamino]-1-(4-isopropoxyphenyl)indole-2-carboxylic acid ethyl ester

5-Benzyloxy-3-(2,2-dimethylpropionylamino)-1-(4-isopropoxy-phenyl)indole-2-carboxylic acid ethyl ester ((1.00 g, 1.89 mmol; see Example 57 step (c)) in DMF (20 mL) was added to a stirred suspension of NaH (67 mg, 2.08 mmol; 75% suspension in mineral oil) in DMF (10 mL) at 0° C. The mixture was stirred at 0° C. for 25 min. A solution of MeI (235 μL, 3.78 mmol) in DMF (10 mL) was added in portions and the mixture was stirred at rt for 24 h, poured into water and extracted with t-BuOMe. The combined extracts were washed with water and brine, dried (Na₂SO₄), concentrated and purified by chromatography to give the sub-title compound (500 mg, 49%).

### (b) 3-[(2,2-Dimethylpropionyl)methylamino]-5-hydroxy-1-(4-isopropoxyphenyl)-indole-2-carboxylic acid ethyl ester

The sub-title product was prepared in accordance to Example 23 step (d) from 5-benzyloxy-3-[(2,2-dimethylpropionyl)methylamino]-1-(4-isopropoxyphenyl)-indole-2-carboxylic acid ethyl ester (see step (a) above).

### (c) 3-(2,2-Dimethylpropionylamino)-1-(4-isopropoxyphenyl)-5-(4-trifluoromethylphenoxy)indole-2-carboxylic acid

The title compound was prepared in accordance with Example 1 step (c) from 3-[(2,2-dimethylpropionyl)methylamino]-5-hydroxy-1-(4-isopropoxyphenyl)-indole-2-carboxylic acid ethyl ester (see step (b) above) and 4-trifluoromethylphenylboronic acid, followed by hydrolysis (see Example 23 step (f)).

200 MHz ¹H-NMR (DMSO-d₆, ppm) δ 7.72-7.62 (2H, m) 7.32-7.19 (3H, m) 7.16-6.96 (6H, m) 4.65 (1H, septet, J=6.0 Hz) 3.12 (3H, s) 1.30 (6H, d, J=6.0 Hz) 0.95 (9H, s).

### Example 61

### 5-(4-Chloro-3-trifluoromethoxyphenoxy)-3-[(2,2-dimethylpropionyl)methylamino]-1-(4-isopropoxyphenyl)indole-2-carboxylic acid

The title compound was prepared in accordance with Example 1 step (c) from 3-[(2,2-dimethylpropionyl)methylamino]-5-hydroxy-1-(4-isopropoxyphenyl)-indole-2-carboxylic acid ethyl ester (see Example 60 step (b)) and 4-chloro-3-trifluoromethoxyphenyl boronic acid (see Example 14, step (b)) followed by hydrolysis (see Example 23 step (f)).

200 MHz ¹H-NMR (DMSO-d₆, ppm) δ 13.4-13.1 (1H, br s) 7.66 (1H, d, J=8.8 Hz) 7.38-7.33 (1H, m) 7.31-7.23 (2H, m) 7.18-7.12 (3H, m) 7.09-7.01 (2H, m) 7.01 (1H, dd, J=8.8, 2.8 Hz) 4.69 (1H, septet, J=6.0 Hz) 3.13 (3H, s) 1.32 (6H, d, J=6.0 Hz) 0.96 (9H, s).

### Example 62

### 3-[(2,2-Dimethylpropionyl)methylamino]-5-(3-fluoro-4-trifluoromethoxyphenoxy)-1-(4-isopropoxyphenyl)indole-2-carboxylic acid

The title compound was prepared in accordance with Example 1 step (c) from 3-[(2,2-dimethylpropionyl)methylamino]-5-hydroxy-1-(4-isopropoxyphenyl)-indole-2-carboxylic acid ethyl ester (see Example 60 step (b)) and 3-fluoro-4-trifluoromethoxyphenylboronic acid (see Example 36, step (a)) followed by hydrolysis (see Example 23 step (f)).

200 MHz ¹H-NMR (DMSO-d₆, ppm) δ 13.4-13.1 (1H, br s) 7.60-7.48 (1H, m) 7.38-7.33 (1H, m) 7.32-7.22 (2H, m) 7.17-7.08 (3H, m) 7.08-7.00 (2H, m) 6.82 (1H, ddd, J=9.0, 2.8, 1.5 Hz) 4.69 (1H, septet, J=6.0 Hz) 3.15 (3H, s) 1.33 (6H, d, J=6.0 Hz) 0.97 (9H, s).

### Example 63

### 3-Acetylamino-1-(4-isopropoxyphenyl)-5-(4-trifluoromethoxyphenoxy)indole-2-carboxylic acid

### (a) 3-Acetylamino-5-benzyloxy-1-(4-isopropoxyphenyl)indole-2-carboxylic acid ethyl ester

The subtitle product was prepared in accordance with Example 52 step (b) from 5-benzyloxy-3-iodo-1-(4-isopropoxyphenyl)indole-2-carboxylic acid ethyl ester (see Example 57 step (b)) and acetamide.

### (b) 3-(Acetyl-tert-butoxycarbonylamino)-5-benzyloxy-1-(4-isopropoxyphenyl)-indole-2-carboxylic acid ethyl ester

(Boc)₂O (1.09 g, 5.87 mmol) and DMAP (144 mg, 1.17 mmol) were added to a stirred suspension of 3-acetylamino-5-benzyloxy-1-(4-isopropoxyphenyl)indole-2-carboxylic acid ethyl ester (571 mg, 1.17 mmol, see step (a) above), Et₃N (200 μL, 1.17 mmol) in CH₂Cl₂. The mixture was stirred at 40° C. for 24 h, poured into HCl (aq, 0.5 M) and extracted with CH₂Cl₂. The combined extracts were washed with NaHCO₃ (aq, sat) and water, dried (Na₂SO₄), concentrated and purified by chromatography to give the sub-title compound (587 mg, 88%).

### (c) 3-(Acetyl-tert-butoxycarbonylamino)-5-hydroxy-1-(4-isopropoxyphenyl)-indole-2-carboxylic acid ethyl ester

The sub-title product was prepared in accordance to Example 23 step (d) from 3-(acetyl-tert-butoxycarbonylamino)-5-benzyloxy-1-(4-isopropoxyphenyl)indole-2-carboxylic acid ethyl ester (see step (b) above).

### (d) 3-(Acetyl-tert-butoxycarbonylamino)-1-(4-isopropoxyphenyl)-5-(4-trifluoromethoxyphenoxy)indole-2-carboxylic acid ethyl ester

The sub-title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see step (c) above) and 4-trifluoromethoxyphenylboronic acid.

### (e) 3-Acetylamino-1-(4-isopropoxyphenyl)-5-(4-trifluoromethoxyphenoxy)indole-2-carboxylic acid ethyl ester

HCl (4 M in dioxane, 0.35 mL, 0.36 mmol) was added to a stirred solution of 3-(acetyl-tert-butoxycarbonylamino)-1-(4-isopropoxyphenyl)-5-(4-trifluoromethoxyphenoxy)indole-2-carboxylic acid ethyl ester (231 mg, 0.36 mmol) in CH₂Cl₂ (10 mL). The mixture was stirred at rt for 2 h. HCl (aq, conc, 0.3 mL) was added and stirring was continued for 2 h. The volatiles were removed and water (20 mL) was added. The mixture was extracted with EtOAc. The combined extracts were washed with NaHCO₃ (aq, sat) and water, dried (Na₂SO₄), concentrated and purified by chromatography to give the sub-title compound (170 mg, 87%).

### (f) 3-Acetylamino-1-(4-isopropoxyphenyl)-5-(4-trifluoromethoxyphenoxy)indole-2-carboxylic acid

The title compound was prepared in accordance with Example 23 step (f) from 3-acetylamino-1-(4-isopropoxyphenyl)-5-(4-trifluoromethoxyphenoxy)indole-2-carboxylic acid ethyl ester (see step (e) above).

200 MHz ¹H-NMR (DMSO-d₆, ppm) δ 0.6 (1H, s) 7.48 (1H, s) 7.24 (2H, d, J=8.8 Hz) 7.23 (2H, d, J=8.8 Hz) 7.07-6.96 (6H, m) 4.67 (1H, septet, J=6.0 Hz) 2.08 (3H, s) 1.32 (6H, d, J=6.0 Hz).

### Example 64

### 3-Acetylamino-1-(4-isopropoxyphenyl)-5-(4-trifluoromethylphenoxy)indole-2-carboxylic acid

The title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see Example 63 step (c)) and 4-trifluoromethylphenylboronic acid, followed by the removal of the Boc-group (see Example 63 step (e)) and hydrolysis (see Example 23 step (f)).

200 MHz ¹H-NMR (DMSO-d₆, ppm) δ 10.61 (1H, s) 7.74-7.63 (3H, m) 7.21 (2H, d, J=8.8 Hz) 7.06 (2H, d, J=8.8 Hz) 7.03-6.95 (4H, m) 4.66 (1H, septet, J=6.0 Hz) 2.08 (3H, s) 1.32 (6H, d, J=6.0 Hz).

### Example 65

### 3-Acetylamino-5-(2,2-difluorobenzo[1,3]dioxol-5-yloxy)-1-(4-isopropoxyphenyl)-indole-2-carboxylic acid

The title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see Example 63 step (c)) and 2,2-difluorobenzo[1,3]dioxole-5-boronic acid (see Example 35 step (a)), followed by removal of the Boc-group (see Example 63 step (e)) and hydrolysis (see Example 23 step (f)).

200 MHz ¹H-NMR (DMSO-d₆, ppm) δ 9.79 (1H, br s) 7.36 (1H, d, J=9.0 Hz) 7.34-7.30 (1H, m) 7.28-7.19 (2H, m) 7.16 (1H, d, J=2.4 Hz) 7.07-6.99 (4H, m) 6.72 (1H, dd, J=9.0, 2.4 Hz) 4.68 (1H, septet, J=6.0 Hz) 2.08 (3H, s) 1.32 (6H, d, J=6.0 Hz).

### Example 66

### 3-Acetylamino-5-(4-chloro-3-trifluoromethoxyphenoxy)-1-(4-isopropoxyphenyl)-indole-2-carboxylic acid

The title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see Example 63 step (c)) and 4-chloro-3-trifluoromethoxyphenyl boronic acid (see Example 14, step (b)), followed by removal of the Boc-group (see Example 63 step (e)) and hydrolysis (see Example 23 step (f)).

200 MHz ¹H-NMR (DMSO-d₆, ppm) δ 9.84-9.74 (1H br s) 7.64 (1H, d, J=9.0 Hz) 7.43-7.39 (1H, m) 7.30-7.21 (2H, m) 7.20-7.16 (1H, m) 7.10-7.00 (4H, m) 6.95 (1H, dd, J=9.0, 2.8 Hz) 4.68 (1H, septet, J=6.0 Hz) 2.09 (3H, s) 1.32 (6H, d, J=6.0 Hz).

### Example 67

### 3-Acetylamino-1-(4-isopropoxyphenyl)-5-(4-isopropoxy-3-trifluoromethoxyphenoxy)indole-2-carboxylic acid

The title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see Example 63 step (c)) and 4-isopropoxy-3-trifluoromethoxyphenyl boronic acid (Example 34 step (a-c)), followed by removal of the Boc-group (see Example 63 step (e)) and hydrolysis (see Example 23 step (f)).

200 MHz ¹H-NMR (DMSO-d₆, ppm) δ 13.2-12.7 (1H, br s) 10.0 (1H, br s) 7.39-7.34 (1H, m) 7.28-7.18 (3H, m) 7.07-6.96 (5H, m) 6.91 (1H, dd, J=9.0, 2.9 Hz) 4.67 (1H, septet, J=6.0 Hz) 4.59 (1H, septet, J=6.0 Hz) 2.07 (3H, s) 1.32 (6H, d, J=6.0 Hz) 1.25 (6H, d, J=6.0 Hz).

### Example 68

### 3-Acetylamino-5-(benzo[1,3]dioxol-5-yloxy)-1-(4-isopropoxyphenyl)indole-2-carboxylic acid

(a) Benzo[1,3]dioxole-5-boronic acid

The sub-title compound was prepared in accordance with Example 14 step (b) from 5-bromobenzo[1,3]dioxole.

(b) The title compound was prepared in accordance with Example 1 step (c) from 3-(acetyl-tert-butoxycarbonylamino)-5-hydroxy-1-(4-isopropoxyphenyl)indole-2-carboxylic acid ethyl ester (see Example 63 step (c)) and benzo[1,3]dioxole-5-boronic acid (see step (a) above), followed by removal of the Boc-group (see Example 63 step (e)) and hydrolysis (see Example 23 step (f)).

200 MHz ¹H-NMR (DMSO-d₆, ppm) δ 11.1-10.9 (1H, br s) 7.65 (1H, s) 7.19-7.10 (2H, m) 7.03-6.93 (2H, m) 6.91-6.87 (2H, m) 6.84 (1H, d, J=8.4 Hz) 6.61 (1H, d, J=2.5 Hz) 6.35 (1H, dd, J=8.4, 2.5) 6.0 (2H, s) 4.64 (1H, septet, J=6.0 Hz) 2.07 (3H, s) 1.31 (6H, d, J=6.0 Hz).

### Example 69

### 3-(Acetylmethylamino)-1-(4-isopropoxyphenyl)-5-(4-trifluoromethylphenoxy)-indole-2-carboxylic acid

### (a) 3-(Acetylmethylamino)-5-hydroxy-1-(4-isopropoxyphenylindole-2-carboxylic acid ethyl ester

The sub-title compound was prepared in accordance Example 60 step (a) and (b) from 3-acetylamino-5-benzyloxy-1-(4-isopropoxyphenyl)indole-2-carboxylic acid ethyl ester (see Example 63 step (a)).

### (b) 3-(Acetylmethylamino)-1-(4-isopropoxyphenyl)-5-(4-trifluoromethylphenoxy)indole-2-carboxylic acid

The title compound was prepared in accordance with Example 1 step (c) from 3-(acetylmethylamino)-5-hydroxy-1-(4-isopropoxyphenyl)indole-2-carboxylic acid ethyl ester (see step (a) above) and 4-trifluoromethylphenylboronic acid followed by hydrolysis (see Example 23 step (f)).

200 MHz ¹H-NMR (DMSO-d₆, ppm) δ 7.76-7.65 (2H, m) 7.42-7.25 (3H, m) 7.21-6.98 (6H, m) 4.69 (1H, septet, J=6.0 Hz) 3.15 (3H, s) 1.79 (3H, s) 1.32 (6H, d, J=6.0 Hz).

### Example 70

### 3-(Acetylmethylamino)-1-(4-isopropoxyphenyl)-5-(4-trifluoromethoxyphenoxy)-indole-2-carboxylic acid

The title compound was prepared in accordance with Example 1 step (c) from 3-(acetylmethylamino)-5-hydroxy-1-(4-isopropoxyphenyl)indole-2-carboxylic acid ethyl ester (see Example 69 step (a)) and 4-trifluoromethoxylphenylboronic acid followed by hydrolysis (see Example 23 step (f)).

200 MHz ¹H-NMR (DMSO-d₆, ppm) δ 7.75-7.66 (2H, m) 7.39-7.29 (3H, m) 7.17-7.07 (4H, m) 7.07-6.99 (2H, m) 4.69 (1H, septet, J=6.0 Hz) 3.16 (3H, s) 1.80 (3H, s) 1.33 (6H, d, J=6.0 Hz).

### Example 71

### 3-(Acetylmethylamino)-5-(benzo[1,3]dioxol-5-yloxy)-1-(4-isopropoxyphenyl)-indole-2-carboxylic acid

The title compound was prepared in accordance with Example 1 step (c) from 3-(acetylmethylamino)-5-hydroxy-1-(4-isopropoxyphenyl)indole-2-carboxylic acid ethyl ester (see Example 69 step (a)) and benzo[1,3]dioxole-5-boronic acid (see Example 68 step (a)) followed by hydrolysis (see Example 23 step (f)).

200 MHz ¹H-NMR (DMSO-d₆, ppm) δ 3.2 (1H, s) 7.36-7.25 (2H, m) 7.15-6.98 (5H, m) 6.88 (1H, d, J=8.4 Hz) 6.73 (1H, d, J=2.4 Hz) 6.46 (1H, dd, J=8.4, 2.4 Hz) 6.03 (2H, s) 4.63 (1H, septet, J=6.0 Hz) 3.13 (3H, s) 1.77 (3H, s) 1.32 (6H, d, J=6.0 Hz).

### Example 72

### 3-(Acetylmethylamino)-5-(4-chloro-3-trifluoromethoxyphenoxy)-1-(4-isopropoxyphenyl)indole-2-carboxylic acid

The title compound was prepared in accordance with Example 1 step (c) from 3-(acetylmethylamino)-5-hydroxy-1-(4-isopropoxyphenyl)indole-2-carboxylic acid ethyl ester (see Example 69 step (a)) and 4-chloro-3-trifluoromethoxyphenyl boronic acid (see Example 14, step (b)) followed by hydrolysis (see Example 23 step (f).

200 MHz ¹H-NMR (DMSO-d₆, ppm) δ 7.64 (1H, d, J=9.0 Hz) 7.36-7.27 (2H, m) 7.25 (1H, d, J=0.9 Hz) 7.19-7.12 (2H, m) 7.08-6.97 (4H, m) 4.67 (1H, septet, J=6.0 Hz) 3.14 (3H, s) 1.79 (3H, s) 1.32 (6H, d, J=6.0 Hz).

### Example 73

### 1-(4-Isopropoxyphenyl)-3-[(pyridine-3-carbonyl)amino]-6-(3-trifluoromethoxyphenoxy)indole-2-carboxylic acid

### (a) 3-Bromo-1-(4-isopropoxyphenyl)-6-(3-trifluoromethoxyphenoxy)indole-2-carboxylic acid ethyl ester

The sub-title compound was prepared in accordance with Example 52 step (a) from 1-(4-isopropoxyphenyl)-6-(3-trifluoromethoxyphenoxy)indole-2-carboxylic acid ethyl ester (see Example 24) followed by bromination with NBS (see Example 52 step (a)).

### (b) 1-(4-Isopropoxyphenyl)-3-[(pyridine-3-carbonyl)amino]-6-(3-trifluoromethoxyphenoxy)indole-2-carboxylic acid

The title compound was prepared in accordance with Example 53 step (a) from 3-bromo-1-(4-isopropoxyphenyl)-6-(3-trifluoromethoxyphenoxy)indole-2-carboxylic acid ethyl ester (see step (a) above) followed by hydrolysis (see Example 23 step (f)).

200 MHz ¹H-NMR (DMSO-d₆, ppm) δ 13.1 (1H, br s) 10.59 (1H, s) 9.21 (1H, d, J=1.2 Hz) 8.83-8.78 (1H, m) 8.42-8.34 (1H, m) 7.86 (1H, d, J=8.8 Hz) 7.62 (1H, ddd, J=8.0, 4.8, 0.5 Hz) 7.39-7.30 (2H, m) 7.29-7.21 (2H, m) 7.12-6.98 (4H, m) 6.95 (1H, dd, J=8.8, 2.0 Hz) 6.60 (1H, d, J=2.0 Hz) 4.66 (1H, septet, J=6.0 Hz) 1.28 (6H, d, J=6.0 Hz).

### Example 74

### 3-(2,2-Dimethylpropionylamino)-1-(4-isopropoxyphenyl)-6-(3-trifluoromethylphenoxy)indole-2-carboxylic acid

### (a) 3-Bromo-1-(4-isopropoxyphenyl)-6-(3-trifluoromethylphenoxy)indole-2-carboxylic acid ethyl ester

The sub-title compound was prepared in accordance with Example 1 step (c) from 6-hydroxy-1-(4-isopropoxyphenyl)indole-2-carboxylic acid ethyl ester (see Example 23 step (d)) and 3-trifluoromethylphenylboronic acid followed by bromination with NBS (see Example 52 step (a)).

### (b) 3-(2,2-Dimethylpropionylamino)-1-(4-isopropoxyphenyl)-6-(3-trifluoromethylphenoxy)indole-2-carboxylic acid

The title compound was prepared in accordance with Example 53 step (a) from 3-bromo-1-(4-isopropoxyphenyl)-6-(3-trifluoromethylphenoxy)indole-2-carboxylic acid ethyl ester (see step (a) above) and 2,2-dimethylpropionamide followed by hydrolysis (see Example 23 step (f)).

200 MHz ¹H-NMR (DMSO-d₆, ppm) δ 13.1 (1H, br s) 9.74 (1H, s) 7.93 (1H, d, J=8.8 Hz) 7.63-7.52 (1H, m) 7.47-7.40 (1H, m) 7.28-7.18 (4H, m) 7.03-6.95 (2H, m) 6.91 (1H, dd, J=8.8, 2.2 Hz) 6.59 (1H, d, J=2.2 Hz) 4.64 (1H, septet, J=6.0 Hz) 1.30 (9H, s) 1.29 (6H, d, J=6.0 Hz).

### Example 75

### 3-(2-Cyanoethyl)-1-(4-isopropoxyphenyl)-5-(5-trifluoromethylpyridin-2-yloxy)indole-2-carboxylic acid

### (a) 5-Benzyloxy-3-(2-cyanovinyl)-1-(4-isopropoxyphenyl)indole-2-carboxy-lic acid ethyl ester

A mixture of 5-benzyloxy-3-iodo-1-(4-isopropoxyphenyl)indole-2-carboxylic acid ethyl ester (2.2 g, 3.96 mmol; see Example 57, step (b)), NaOAc (1.3 g, 16 mmol), PdCl₂(PPh₃)₂ (140 mg, 0.2 mmol), acrylonitrile (1.1 ml, 16 mmol), Et₃N (0.7 mL, 5 mmol) and DMF (10 mL) was stirred under argon at 70° C. for 7 h. The mixture was allowed to cool to rt, diluted with EtOAc, washed with water, brine and NaHCO₃ (aq, sat), dried (Na₂SO₄), concentrated and purified by chromatography to give the sub-title compound (1.72 g, 90%).

### (b) 3-(2-Cyanoethyl)-5-hydroxy-1-(4-isopropoxyphenyl)indole-2-carboxylic acid ethyl ester

The sub-title compound was prepared in accordance with Example 23 step (d) from 5-benzyloxy-3-(2-cyanovinyl)-1-(4-isopropoxyphenyl)indole-2-carboxylic acid ethyl ester (see step (a) above).

### (c) 3-(2-Cyanoethyl)-1-(4-isopropoxyphenyl)-5-(5-trifluoromethylpyridin-2-yl-oxy)indole-2-carboxylic acid ethyl ester

A mixture of 3-(2-cyanoethyl)-5-hydroxy-1-(4-isopropoxyphenyl)indole-2-carboxylic acid ethyl ester (100 mg, 0.25 mmol, see step (b) above), 2-chloro-5-trifluoromethylpyridine (49 mg, 0.26 mol), K₂CO₃ (173 mg, 1.25 mmol), 18-crown-6 (7 mg, 0.025 mmol) and DMF (2 mL) was stirred at 50° C. for 40 hours. The mixture was diluted with EtOAc, washed with NaHCO₃ (aq, sat) and dried (Na₂SO₄). Concentration and purification by chromatography gave the sub-title compound (110 mg, 80%).

### (d) 3-(2-Cyanoethyl)-1-(4-isopropoxyphenyl)-5-(5-trifluoromethylpyridin-2-yl-oxy)indole-2-carboxylic acid

The title compound was prepared in accordance with Example 23 step (f) from 3-(2-cyanoethyl)-1-(4-isopropoxyphenyl)-5-(5-trifluoromethylpyridin-2-yloxy)-indole-2-carboxylic acid ethyl ester (see step (c) above).

200 MHz ¹H-NMR (DMSO-d₆, ppm) δ 8.55 (1H, br s) 8.23 (1H, dd, J=8.9, 2.3 Hz) 7.76 (1H, d, J=2.0 Hz) 7.30-7.18 (3H, m) 7.13 (1H, dd, J=9.0 Hz) 7.08-6.98 (3H, m) 4.68 (1H, septet, J=6.0 Hz) 3.45-3.29 (m, 2H, overlapped with water) 2.82 (1H, t, J=7.3 Hz) 1.33 (6H, d, J=6.0 Hz)

### Example 76

### 5-(6-Chloropyridin-2-yloxy)-3-(2-cyanoethyl)-1-(4-isopropoxyphenyl)indole-2-carboxylic acid

The title compound was prepared in accordance with Example 75 step (c) from 3-(2-cyanoethyl)-5-hydroxy-1-(4-isopropoxyphenyl)indole-2-carboxylic acid ethyl ester and 2,6-dichloropyridine (see Example 75 step (b)), followed by hydrolysis (see Example 23, step (f)).

200 MHz ¹H-NMR (CDCl₃, ppm) δ 7.62 (1H, t, J=7.8 Hz) 7.54-7.46 (1H, m) 7.27-6.92 (7H, m) 6.78 (1H, d, J=8.4 Hz) 4.63 (1H, septet, J=6.0 Hz) 3.48 (2H, t, J=7.3 Hz) 2.78 (2H, t, J=7.3 Hz) 1.41 (6H, d, J=6.0 Hz)

### Example 77

### 1-(4-Isopropoxyphenyl)-3-(2-oxopyrrolidin-1-yl)-5-(5-trifluoromethylpyridin-2-yloxy)indole-2-carboxylic acid

### (a) 5-Hydroxy-1-(4-isopropoxyphenyl)-3-(2-oxopyrrolidin-1-yl)indole-2-carboxylic acid ethyl ester

The sub-title compound was prepared in accordance with Example 53, step (a) from 5-benzyloxy-3-iodo-1-(4-isopropoxyphenyl)indole-2-carboxylic acid ethyl ester (Example 57, step (b)), followed by removal of the O-benzyl group (see Example 23, step (d)).

### (b) 1-(4-Isopropoxyphenyl)-3-(2-oxopyrrolidin-1-yl)-5-(5-trifluoromethylpyridin-2-yloxy)indole-2-carboxylic acid

The title compound was prepared in accordance with Example 75, step (c) from 5-hydroxy-1-(4-isopropoxyphenyl)-3-(2-oxopyrrolidin-1-yl)indole-2-carboxylic acid ethyl ester (see step (a) above) and 2-chloro-5-trifluoromethylpyridine, followed by hydrolysis (see Example 23, step (f)).

200 MHz ¹H-NMR (DMSO-d₆, ppm) δ 8.52 (1H, s) 8.27-8.14 (1H, m) 7.40-6.94 (8H, m) 4.67 (1H, septet, J=5.9 Hz); 3.93-3.75 (2H, m) 2.6-2.3 (2H, m, overlapped with DMSO) 2.21-2.02 (2H, m) 1.32 (6H, d, J=5.9 Hz).

### Example 78

### 5-(6-Chloropyridin-2-yloxy)-1-(4-isopropoxyphenyl)-3-(2-oxopyrrolidin-1-yl)-indole-2-carboxylic acid

The title compound was prepared in accordance with Example 75, step (c) from 5-hydroxy-1-(4-isopropoxyphenyl)-3-(2-oxopyrrolidin-1-yl)indole-2-carboxylic acid ethyl ester and 2,6-dichloropyridine (see Example 77, step (a)), followed by hydrolysis (see Example 23, step (f)).

200 MHz ¹H-NMR (DMSO-d₆, ppm) δ 7.92-7.81 (1H, m) 7.37-7.16 (4H, m) 7.13-6.91 (m, 5H) 4.68 (1H, septet, J=5.9 Hz) 3.83 (2H, t, J=6.7 Hz) 2.55-2.30 (2H, m, overlapped with DMSO) 2.23-2.05 (2H, m) 1.32 (6H, d, J=5.9 Hz).

### Example 79

### 3-Chloro-1-(4-cyclopentyloxyphenyl)-5-phenylethynylindole-2-carboxylic acid

### (a) 3-Chloro-1-(4-cyclopentyloxyphenyl)-5-phenylethynylindole-2-carboxylic acid ethyl ester

A mixture of 5-bromo-3-chloro-1-(4-cyclopentyloxyphenyl)indole-2-carboxylic acid ethyl ester (690 mg, 1.5 mmol; see Example 39, step (c)), trimethylphenylethynylstannane (776 mg, 3.0 mg), Pd[(PPh₃)]₄ (27 mg, 0.023 mmol), Ph₃P (6.0 mg, 0.023 mmol) and anhydrous toluene (4.0 mL) was heated under argon at 110° C. for 12 h, whereupon the color changed from cloudy yellow to black. After dilution with EtOAc (30 mL), the mixture was washed with NH₄Cl (aq, 10%), brine and dried (Na₂SO₄). Concentration and purification by chromatography afforded the sub-title compound (300 mg, 42% yield).

### (b) 3-Chloro-1-(4-cyclopentyloxyphenyl)-5-phenylethynyl indole-2-carboxylic acid

The title compound was prepared in accordance with Example 23, step (f) from 3-chloro-1-(4-cyclopentyloxyphenyl)-5-phenylethynyl indole-2-carboxylic acid ethyl ester (see step (a) above).

200 MHz ¹H-NMR (DMSO-d₆, ppm) δ 13.55-13.35 (1H, br s) 7.90 (1H, d, J=1.1 Hz) 7.63-7.42 (6H, m) 7.36-7.30 (2H, m) 7.10 (1H, d, J=8.7 Hz) 7.07-7.00 (2H, m) 4.95-4.86 (1H, m) 2.03-1.57 (8H, m).

### Example 80

### 3-Chloro-1-(4-isopropoxyphenyl)-5-piperidin-1-ylindole-2-carboxylic acid

### (a) 5-Bromo-3-chloroindole-2-carboxylic acid ethyl ester

A mixture of 5-bromoindole-2-carboxylic acid ethyl ester (4.00 g, 14.9 mmol), SO₂Cl₂ (1.8 mL, 22.4 mmol) and benzene (125 mL) was stirred at 90° C. for 2.5 h, and cooled to rt. NaHCO₃ (aq, sat) was added and the mixture was extracted with EtOAc. The combined extracts were washed with water and brine and dried (Na₂SO₄). Concentration and crystallisation from toluene gave the sub-title compound (3.87 g 85%).

### (b) 5-Bromo-3-chloro-1-(4-isopropoxyphenyl)indole-2-carboxylic acid ethyl ester

Anhydrous CH₂Cl₂ (80 mL), Et₃N (3.36 mL, 23.9 mmol) and pyridine (1.95 mL, 23.9 mmol) were added to 5-bromo-3-chloroindole-2-carboxylic acid ethyl ester (3.60 g, 11.9 mmol; see step (a) above), Cu(OAc)₂ (4.34 g, 23.9 mmol), 3 Å molecular sieves (ca. 7 g) and 4-cyclopentyloxyphenylboronic acid (4.30 g, 23.9 mmol). The mixture was stirred vigorously at rt for 48 h, and additional Et₃N (1.6 mL, 11.0 mmol), pyridine (0.90 mL, 11.0 mmol), Cu(OAc)₂ (2.00 g, 11.0 mmol) and 4-cyclopentyloxyphenylboronic acid (2.27 g, 11.0 mmol) were added. The mixture was stirred at rt for 48 h and filtered through Celite®. The solids were washed with EtOAc and the combined filtrates were washed with NH₄OH (aq), HCl (aq, 0.1 M) and brine, dried (Na₂SO₄), concentrated and purified by chromatography to afford the sub-title compound (4.40 g, 85%).

### (c) 3-Chloro-1-(4-isopropoxyphenyl)-5-piperidin-1-ylindole-2-carboxylic acid ethyl ester

A mixture of 5-bromo-3-chloro-1-(4-isopropoxyphenyl)indole-2-carboxylic acid ethyl ester (198 mg, 0.45 mmol) (step (b) above), Pd₂(dba)₃ (20.6 mg, 0.023 mmol), BINAP (42 g, 0.068 mmol), piperazine (55 μL, 0.56 mmol) Cs₂CO₃ (205 mg, 0.63 mmol) and toluene (2 mL) was stirred at 80° C. for 24 h. The mixture was cooled to rt and filtered through Celite® and the solids were washed with EtOAc. The combined filtrates were washed with water, brine, dried (Na₂SO₄), concentrated and purified by chromatography to afford the sub-title compound (75 mg, 37%).

### (d) 3-Chloro-1-(4-isopropoxyphenyl)-5-piperidin-1-ylindole-2-carboxylic acid

A mixture of 3-chloro-1-(4-isopropoxyphenyl)-5-piperidin-1-ylindole-2-carboxylic acid ethyl ester (75 mg, 0.17 mmol; see step (c)), NaOH (34 mg, 0.85 mmol), water (1.0 mL) and EtOH (2.0 mL) was stirred at 120° C. for 30 min. After cooling, the mixture was acidified with HCl (aq, 1 M) to pH 5 and extracted with EtOAc. The combined extracts were washed with brine and dried (Na₂SO₄). Concentration and purification by chromatography gave the title compound (60 mg, 85%).

200 MHz ¹H-NMR (DMSO-d₆, ppm) δ 13.1 (1H, br s) 7.26-7.16 (2H, m) 7.12 (1H, dd, J=9.2, 2.0 Hz) 7.06-6.95 (2H, m) 6.93 (1H, d, J=2.0 Hz) 6.90 (1H, d, J=9.2 Hz) 4.64 (1H, septet, J=6.0 Hz) 3.12-3.00 (4H, m) 1.72-1.42 (6H, m) 1.30 (6H, d, J=6.0 Hz).

### Example 81

### 5-(5-tert-Butyl-2-oxocyclohexyl)-3-chloro-1-(4-isopropoxyphenyl)indole-2-carboxylic acid

### (a) 5-(5-tert-Butyl-2-oxocyclohexyl)-3-chloro-1-(4-isopropoxyphenyl)indole-2-carboxylic acid ethyl ester

To a mixture of 5-bromo-3-chloro-1-(4-isopropoxyphenyl)indole-2-carboxylic acid ethyl ester (600 mg, 1.37 mmol) (step (b) in Example 80), 4-tert-butylcyclohexanone (848 mg, 5.5 mmol) and K₃PO₄ (1.20 g, 5.6 mmol) and toluene (0.3 mL), was added a solution of Pd₂(dba)₃ (6.18 mg, 0.0068 mmol) and xantphos (7.81 mg, 0.0136 mmol) in toluene (0.3 mL). The mixture was stirred at 80° C. for 23 h, cooled to rt, diluted with EtOAc, washed with water and brine, dried (Na₂SO₄), concentrated and purified by chromatography to afford the sub-title compound (292 mg, 41%).

### (b) 5-(5-tert-Butyl-2-oxocyclohexyl)-3-chloro-1-(4-isopropoxyphenyl)indole-2-carboxylic acid

A mixture of 5-(5-tert-butyl-2-oxocyclohexyl)-3-chloro-1-(4-isopropoxyphenyl)-indole-2-carboxylic acid ethyl ester (290 mg, 0.57 mmol; see step (a) above), NaOH (136 mg, 3.41 mmol), water (60 mL) and EtOH (40 mL) was stirred at reflux for 2 h. After cooling, the EtOH was partly evaporated and the mixture was acidified with HCl (aq, 1M) to pH 5 and extracted with EtOAc. The combined extracts were washed with brine and dried (Na₂SO₄). Concentration and purification by chromatography gave the title compound (165 mg, 60%).

200 MHz ¹H-NMR (DMSO-d₆, ppm) δ for the major diastereomer 13.3 (1H, br s) 7.44 (1H, s) 7.28-7.24 (2H m) 7.13 (1H, d, J=8.7 Hz) 7.05-7.01 (2H, m) 6.97 (1H, d, J=8.7 Hz) 4.68 (1H, septet, J=6.0 Hz) 3.97-3.92 (1H, m) 2.58 (1H, td, J=14.0, 6.0 Hz) 2.36-2.30 (1H, m) 2.15-2.03 (2H, m) 1.84-1.76 (2H, m) 1.62-1.54 (1H, m) 1.32 (6H, d, J=6.0 Hz) 0.92 (9H, s)

### Example 82

### 5-(5-tert-Butyl-2-hydroxycyclohexyl)-3-chloro-1-(4-isopropoxyphenyl)indole-2-carboxylic acid

NaBH₄ (92 mg, 1.0 mmol) was added in portions to a mixture of 5-(5-tert-butyl-2-oxo-cyclohexyl)-3-chloro-1-(4-isopropoxyphen-yl)indole-2-carboxylic acid (102 mg, 0.21 mmol; see step (b) in Example 81) water (6 mL) and EtOH (10 mL). After 20 min the mixture was acidified with HCl (aq, 1 M) to pH 1 and stirred for an additional 60 min. The EtOH was partly evaporated and the mixture was extracted with EtOAc. The combined extracts were washed with brine and dried (Na₂SO₄) Concentration and purification by chromatography gave the title compound (165 mg, 60%).

200 MHz ¹H-NMR (DMSO-d₆, ppm) 6 for the major diastereomer 13.2-13.0 (1H, br s) 7.48 (1H, s) 7.32-7.20 (3H, m) 7.08-6.92 (3H, m) 4.68 (1H, septet, J=6.0 Hz) 4.30-4.23 (1H, m) 3.58-3.50 (1H, m) 2.04-2.67 (1H, m) 1.80-1.70 (2H, m) 1.58-1.37 (1H, m) 1.32 (6H, d, J=6.0 Hz) 1.30-1.10 (4H, m) 0.84 (9H, s)

### Example 83

### 3-Chloro-1-(4-isopropoxyphenyl)-5-(2-phenylcyclopropyl)indole-2-carboxylic acid

### (a) 4,4,5,5-Tetramethyl-2-(2-phenylcyclopropyl)-[1,3,2]dioxaborolane

Diazomethane (2 g, 47 mmol) in Et₂O (100 mL) was added over 2 h to 4,4,5,5-tetramethyl-2-((E)-styryl)-[1,3,2]dioxaborolane (0.8 g, 3.5 mmol), Pd(OAc)₂ (45 mg, 0.2 mmol) and Et₂O (1.0 mL) at 0° C. The mixture was stirred for 2 h at rt, filtered through Celite®, concentrated and purified by chromatography to afford the sub-title compound (625 mg, 80%).

### (b) Potassium 2-phenyl-cyclopropyltrifluoroborate

A mixture of 4,4,5,5-tetramethyl-2-(2-phenylcyclopropyl)-[1,3,2]dioxaborolane (300 mg, 1.23 mmol; see step (a) above), KHF₂ (670 mg, 8.6 mmol), water (1 mL) and MeOH (4 mL) was stirred at rt for 4 h. The mixture was concentrated and the residue treated with MeCN. The mixture was filtered and concentrated. The residue was treated with Et₂O and filtered to afford 224 mg (81%) of the sub-title compound.

### (c) 3-Chloro-1-(4-isopropoxyphenyl)-5-(2-phenylcyclopropyl)indole-2-carboxylic acid ethyl ester

A mixture of 5-bromo-3-chloro-1-(4-isopropoxyphenyl)indole-2-carboxylic acid ethyl ester (218 mg, 0.5 mmol) (step (b) in Example 80), potassium 2-phenylcyclopropyltrifluoroborate (132 mg, 0.6 mmol) (step (b) above), Pd(PPh₃)₄ (29 mg, 0.025 mmol), K₃PO₄ (254 mg, 1.23 mmol), toluene (1.5 mL) and water was stirred at 110° C. for 17 h. The mixture was cooled to rt, diluted with EtOAc and washed with HCl (aq, 0.1 M), NaHCO₃ (aq, sat), water, brine and dried (Na₂SO₄). Concentration and purification by chromatography gave the sub-title compound (74 mg, 31%).

### (d) 3-Chloro-1-(4-isopropoxyphenyl)-5-(2-phenylcyclopropyl)indole-2-carboxylic acid

The title compound was prepared in accordance with step ((b) in Example 81) from 3-chloro-1-(4-isopropoxyphenyl)-5-(2-phenylcyclopropyl)indole-2-carboxylic acid ethyl ester (see step (c) in above).

200 MHz ¹H-NMR (DMSO-d₆, ppm) δ 13.3-13.1 (1H, br s) 7.46 (1H, s) 7.36-7.10 (8H, m) 7.09-6.92 (3H, m) 4.68 (1H, septet, J=6.0 Hz) 2.43-2.30 (1H, m) 2.28-2.13 (1H, m) 1.59-1.40 (2H, m) 1.32 (6H, d, J=6.1 Hz)

### Example 84

The following compounds are prepared in accordance with techniques described herein:
3-chloro-5-cyclohexyl-1-(4-isopropoxyphenyl)indole-2-carboxylic acid;
3-chloro-5-(norbornan-2-yl)-1-(4-isopropoxyphenyl)indole-2-carboxylic acid;
3-chloro-5-cyclopropyl-1-(4-isopropoxyphenyl)indole-2-carboxylic acid;
3-chloro-5-(cyclopenten-1-yl)-1-(4-isopropoxyphenyl)indole-2-carboxylic acid;
3-chloro-5-(5,5-dimethylcyclohexen-3-one-1-yl)-1-(4-isopropoxyphenyl)indole-2-carboxylic acid;
3-chloro-5-(1-tert-butoxycarbonyl-1,2,3,4-tetrahydropyrid-6-yl)-1-(4-isopropoxyphenyl)indole-2-carboxylic acid;
3-chloro-6-cyclohexyl-1-(4-isopropoxyphenyl)indole-2-carboxylic acid;
3-chloro-6-(norbornan-2-yl)-1-(4-isopropoxyphenyl)indole-2-carboxylic acid;
3-chloro-6-cyclopropyl-1-(4-isopropoxyphenyl)indole-2-carboxylic acid;
3-chloro-6-(cyclopenten-1-yl)-1-(4-isopropoxyphenyl)indole-2-carboxylic acid;
3-chloro-6-(5,5-dimethylcyclohexen-3-one-1-yl)-1-(4-isopropoxyphenyl)indole-2-carboxylic acid;
3-chloro-6-(1-tert-butoxycarbonyl-1,2,3,4-tetrahydropyrid-6-yl)-1-(4-isopropoxyphenyl)indole-2-carboxylic acid;
3-chloro-5-(pyrrolidin-1-yl)-1-(4-isopropoxyphenyl)indole-2-carboxylic acid;
3-chloro-5-(morpholin-1-yl)-1-(4-isopropoxyphenyl)indole-2-carboxylic acid;
3-chloro-5-(4-cyclopentylpiperazin-1-yl)-1-(4-isopropoxyphenyl)indole-2-carboxylic acid;
3-chloro-6-(pyrrolidin-1-yl)-1-(4-isopropoxyphenyl)indole-2-carboxylic acid;
3-chloro-6-(morpholin-1-yl)-1-(4-isopropoxyphenyl)indole-2-carboxylic acid; and
3-chloro-6-(4-cyclopentylpiperazin-1-yl)-1-(4-isopropoxyphenyl)indole-2-carboxylic acid.

### Example 85

Title compounds of the examples were tested in the biological test described above and were found to exhibit 50% inhibition of mPGES-1 at a concentration of 10 μM or below. For example, the following representative compounds of the examples exhibited the following IC₅₀ values:

| | |
|---|---|
| Example 1: | 430 nM |
| Example 10: | 240 nM |
| Example 13: | 3700 nM |
| Example 21: | 75 nM |
| Example 40: | 610 nM |

1. A compound of formula I, 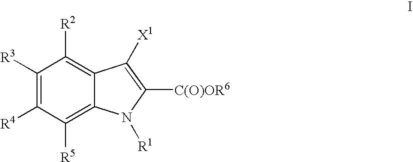
wherein
one of the groups R², R³, R⁴ and R⁵ represents -D-E, a cycloalkyl group or a heterocycloalkyl group (which latter two groups are optionally substituted by one or more substituents selected from G¹ and/or Z¹) and:
a) the other groups are independently selected from hydrogen, G¹, C₁₋₈ alkyl and a heterocycloalkyl group (which latter two groups are optionally substituted by one or more substituents selected from G¹ and/or Z¹) and, in the case when one of R², R³, R⁴ and R⁵ represents -D-E, an aryl group and a heteroaryl group (which latter two groups are optionally substituted by one or more substituents selected from A); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, —OR⁶ and ═O;
D represents —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R⁹)-J-R¹⁰ or -Q-X¹;
J represents a single bond, —C(O)— or —S(O)ₘ—;
Q represents a single bond, —O—, —C(O)— or —S(O)ₘ—;
m represents 0, 1 or 2;
X represents:
(a) an aryl group or a heteroaryl group, both of which are optionally substituted by one or more substituents selected from A; or
(b) C₁₋₈ alkyl or a heterocycloalkyl group, both of which are optionally substituted by one or more substituents selected from G¹ and/or Z¹;
R⁶, R⁹ and R¹⁰ independently represent:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
R⁹ and R¹⁰ may be linked together to form, along with the N atom and the J group to which R⁹ and R¹⁰ are respectively attached, a 3- to 8-membered ring, optionally containing 1 to 3 heteroatoms and/or 1 to 3 double bonds, which ring is optionally substituted by one or more substituents selected from G¹ and/or Z¹;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{11a};
wherein A¹ represents a single bond or a spacer group selected from —C(O)A²-, —S(O)₂A³-, —N(R^{12a})A⁴- or —OA⁵-, in which:
A² represents a single bond, —O—, —N(R^{12b})— or —C(O)—;
A³ represents a single bond, —O— or —N(R^{12c});
A⁴ and A⁵ independently represent a single bond, —C(O)—, —C(O)N(R^{12d})—, —C(O)O—, —S(O)₂— or —S(O)₂N(R^{12e});
Z¹ represents ═O, ═S, ═NOR^{11b}, ═NS(O)₂N(R^{12f})R^{11c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{13a};
wherein A⁶ represents a single bond or a spacer group selected from —C(O)A⁷-, —S(O)₂A⁸-, —N(R⁴a)A⁹- or —OA¹⁰-, in which:
A⁷ represents a single bond, —O—, —N(R^{14b}) or —C(O)—;
A⁸ represents a single bond, —O— or —N(R^{14c})—;
A⁹ and A¹⁰ independently represent a single bond, —C(O)—, —C(O)N(R^{14d})—, —C(O)O—, —S(O)₂— or —S(O)₂N(R^{14e})—;
Z² represents ═O, ═S, ═NOR^{13b}, ═NS(O)₂N(R^{14f})R^{13c}, ═NCN or ═C(H)NO₂;
R^{11a}, R^{11b}, R^{11c}, R^{12a}, R^{12b}, R^{12c}, R^{12d}, R^{12e}, R^{12f}, R^{13a}, R^{13b}, R^{13c}, R^{14a}, R^{14b}, R^{14c}, R^{14d}, R^{14e} and R^{14f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by G³ and/or Z³; or
any pair of R^{1a} to R^{11c} and R^{12a} to R^{12f}, and/or R^{13a} to R^{13c} and R^{14a} to R^{14f}, may be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{15a};
wherein A¹¹ represents a single bond or a spacer group selected from —C(O)A¹²-, —S(O)₂A¹³-, —N(R^{16a})A¹⁴ or —OA¹⁵-, in which:
A¹² represents a single bond, —O—, —N(R^{16b})— or —C(O)—;
A¹³ represents a single bond, —O— or —N(R^{16c})—;
A¹⁴ and A¹⁵ independently represent a single bond, —C(O)—, C(O)N(R^{16d})—, —C(O)O—, —S(O)₂— or —S(O)₂N(R^{16e})—;
Z³ represents ═O, S, ═NOR^{15b}, ═NS(O)₂N(R^{16f})R^{15c}, ═NCN or ═C(H)N₂;
R^{15a}, R^{15b}, R^{15c}, R^{16a}, R^{16b}, R^{16c}R^{16d}, R^{16e} and R^{16f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{17a})R^{18a}, —OR^{17b} and ═O; and
iii) an aryl or heteroaryl group, both of which are optionally substituted by one or more substituents selected from halo, C₁₋₄ alkyl, —N(R^{17c})R^{18b} and —OR^{17d}; or any pair of R^{15a} to R^{15c} and R^{16a} to R^{16f} may be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{17e})R^{18c}, —OR^{17f} and ═O;
R^{17a}, R^{17b}, R^{17c}, R^{17d}, R^{17e}, R^{17f}, R^{18a}, R^{18b} and R^{18c} are independently selected from hydrogen and C₁₋₄ alkyl, which latter group is optionally substituted by one or more halo groups;
or a pharmaceutically-acceptable salt thereof,
provided that, when R³ represents -D-E, in which D represents —C(R⁷)(R⁸)—, X¹, R², R⁴, R⁵, R⁷ and R⁸ all represent H and:
(a) E represents a 2-butyl-5-hydroxymethyl-1H-imidazol-1-yl group, then R⁶ does not represent H when R¹ represents phenyl or 2-carboxyphenyl;
(b) E represents a 2-butyl-5-hydroxymethyl-1H-imidazol-1-yl group or a 2-butyl-5-formyl-1H-imidazol-1-yl group, then R⁶ does not represent ethyl when R¹ represents phenyl or 2-ethoxycarbonylphenyl;
(c) E represents a 2-butyl-4-chloro-5-hydroxymethyl-1H-imidazol-1-yl group, then R⁶ does not represent H or ethyl when R¹ represents 2-(1H-tetrazol-5-yl)phenyl; or
(d) E represents a 2-butyl-4-chloro-5-hydroxymethyl-1H-imidazol-1-yl group or a 2-butyl-4-chloro-5-formyl-1H-imidazol-1-yl group, then R⁶ does not represent ethyl when R¹ represents 2-cyanophenyl.

2. A compound as claimed in claim 1, wherein A represents C₁₋₆ alkyl optionally substituted by one or more G¹ groups or G¹.

3. A compound as claimed in claim 1 or claim 2, wherein G¹ represents halo, cyano, —NO₂ or -A¹-R^{11a}.

4. A compound as claimed in claim 3, wherein, when one of R² to R⁵ represents -D-E-, then G¹ represents fluoro, chloro, —NO₂ or -A¹-R^{11a}.

5. A compound as claimed in claim 3, wherein, when one of R² to R⁵ represents an optionally substituted cycloalkyl or heterocycloalkyl group, then G¹ represents fluoro, chloro or -A¹-R^{11a}.

6. A compound as claimed in claim 1, wherein, when one of R² to R⁵ represents -D-E-, then A¹ represents a single bond, —C(O)O—, —N(R^{12a})A⁴- or —OA⁵-.

7. A compound as claimed in claim 1, wherein when one of R² to R⁵ represents an optionally substituted cycloalkyl or heterocycloalkyl group, then A¹ represents a single bond, —C(O)O—, —C(O)N(R^{12b})—, —N(R^{12a})A⁴- or —OA⁵-.

8. A compound as claimed in claim 7, wherein A¹ represents a single bond, —C(O)O— or —OA⁵-.

9. A compound as claimed in claim 1, wherein A⁴ and A⁵ independently represent a single bond.

10. A compound as claimed in claim 1, wherein, when one of R² to R⁵ represents -D-E-, then R^{11a}, R^{11b} and R^{11c} independently represent a phenyl group, a tetrazolyl group, an imidazolyl group, a pyridyl group, or a C₁₋₃ alkyl group, all of which are optionally substituted by one or more G³ groups.

11. A compound as claimed in claim 1, wherein, when one of R² to R⁵ represents an optionally substituted cycloalkyl or heterocycloalkyl group, then R^{11a}, R^{11b} and R^{11c} independently represent a phenyl group, a tetrazolyl group, a pyridyl group, an imidazolyl group, C₁₋₆ alkyl or C₄₋₆ heterocycloalkyl, all of which are optionally substituted by one or more halo groups.

12. A compound as claimed in claim 11, wherein R^{11a}, R^{11b} and R^{11c} independently represent C₁₋₆ alkyl or C₄₋₆ heterocycloalkyl, both of which are optionally substituted by one or more halo groups.

13. A compound as claimed in claim 1, wherein G³ represents halo.

14. A compound as claimed in claim 1, wherein D represents —CH₂—, ethylene, —S—, —S(O)—, —S(O)₂—, —O— or —C(O)—.

15. A compound as claimed in claim 14, wherein D represents —O— or —C(O)—.

16. A compound as claimed in claim 1, wherein R¹, E and (when they represent such aryl or heteroaryl groups) X², R⁹ and R¹⁰ represent optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

17. A compound as claimed in claim 16, wherein R¹ represents optionally substituted phenyl, pyridyl or imidazolyl.

18. A compound as claimed in claim 16 or claim 17, wherein E represents optionally substituted 1,3-benzodioxolyl, phenyl, pyridyl, quinolinyl or imidazolyl.

19. A compound as claimed in claim 18, wherein E represents phenyl, pyridyl, quinolinyl or imidazolyl.

20. A compound as claimed in claim 16, wherein, when one of R² to R⁵ represents -D-E-, then the optional substituents are selected from phenyl, ═O (in the case of substituents on non-aromatic rings), halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR¹⁹ and —N(R¹⁹)R²⁰, wherein R¹⁹ and R²⁰ independently represent H or C₁₋₆ alkyl (which alkyl group is optionally substituted by one or more halo groups).

21. A compound as claimed in claim 20, wherein the optional substituents are selected from halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR¹⁹ and —N(R¹⁹)R²⁰, wherein R¹⁹ and R²⁰ independently represent H or C₁₋₆ alkyl (which alkyl group is optionally substituted by one or more halo groups).

22. A compound as claimed in claim 16, wherein, when one of R² to R⁵ represents an optionally substituted cycloalkyl or heterocycloalkyl group, then the optional substituents are selected from phenyl, ═O (in the case of substituents on non-aromatic rings), halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR¹⁹, —N(R¹⁹)R²⁰ and —C(O)OR¹⁹, wherein R¹⁹ and R²⁰ independently represent H or C₁₋₆ alkyl (which alkyl group is optionally substituted by one or more halo groups).

23. A compound as claimed in claim 22, wherein the optional substituents are selected from halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR¹⁹, —N(R¹⁹)R²⁰ and —C(O)OR¹⁹, wherein R¹⁹ and R²⁰ independently represent H or C₁₋₆ alkyl (which alkyl group is optionally substituted by one or more halo groups).

24. A compound as claimed in claim 1, wherein X¹ represents —N(R⁹)-J-R¹⁰, C₁₋₃ alkyl, heterocycloalkyl (which latter two groups are optionally substituted by —N(R^{12a})R^{11a}—, —OR^{11a}, —R^{11a} or halo), H or halo.

25. A compound as claimed in claim 24, wherein X¹ represents C₁₋₃ alkyl, heterocycloalkyl (which latter two groups are optionally substituted by —N(R^{12a})R^{11a}, —OR^{11a}, —R^{11a} or halo), H or halo.

26. A compound as claimed in claim 1, wherein J represents —C(O)— or —S(O)₂—.

27. A compound as claimed in claim 1, wherein one of R⁴ and R³ represents -D-E or an optionally substituted cycloalkyl or heterocycloalkyl group and the other represents H.

28. A compound as claimed in claim 27, wherein R³ represents -D-E.

29. A compound as claimed in claim 1, wherein, when one of R² to R⁵ represents -D-E, then R² represents chloro or H.

30. A compound as claimed in claim 1, wherein R² and/or R⁵ independently represent H.

31. A compound as claimed in claim 1, wherein R⁶ represents H.

32. A compound as defined in claim 1, but without provisos (b) and (d), or a pharmaceutically-acceptable salt thereof, for use as a pharmaceutical.

33. A pharmaceutical formulation including a compound as defined in claim 1, but without provisos (b) and (d), or a pharmaceutically-acceptable salt thereof, in admixture with a pharmaceutically acceptable adjuvant, diluent or carrier.

34. (canceled)

35. (canceled)

36. (canceled)

37. A use as claimed in claim 39, wherein the disease is inflammation.

38. A use as claimed in claim 39 wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

39. A method of treatment of a disease in which inhibition of the activity of a member of the MAPEG family is desired and/or required, which method comprises administration of a therapeutically effective amount of a compound as defined in claim 1, but without the provisos, or a pharmaceutically-acceptable salt thereof, to a patient suffering from, or susceptible to, such a condition.

40. A method as claimed in claim 39, wherein the member of the MAPEG family is microsomal prostaglandin E synthase-1, leukotriene C₄ and/or 5-lipoxygenase-activating protein.

41. A method as claimed in claim 40, wherein the member of the MAPEG family is microsomal prostaglandin E synthase-1.

42. A combination product comprising:
(A) a compound as defined in claim 1, but without the provisos, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

43. A combination product as claimed in claim 42 which comprises a pharmaceutical formulation including a compound as defined in claim 1, but without the provisos, or a pharmaceutically-acceptable salt thereof, another therapeutic agent that is useful in the treatment of inflammation, and a pharmaceutically-acceptable adjuvant, diluent or carrier.

44. A combination product as claimed in claim 42 which comprises a kit of parts comprising components:
(a) a pharmaceutical formulation including a compound as defined in claim 1, but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

45. A process for the preparation of a compound as defined in claim 1, which comprises:
(i) reaction of a compound of formula II, 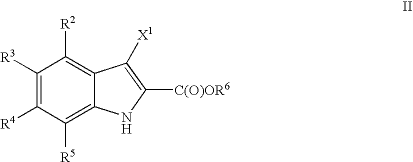
wherein X¹, R², R³, R⁴, R⁵ and R⁶ are as defined in claim 1, with a compound of formula III,
R¹L¹ III
wherein L¹ represents a suitable leaving group and R¹ is as defined in claim 1;
(ii) for compounds of formula I in which X¹ represents -Q-X², in which Q is a single bond or —C(O)—, reaction of a compound of formula IV, 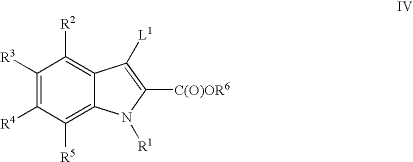
wherein R¹, R², R³, R⁴, R⁵ and R⁶ are as defined in claim 1 and L¹ is as defined above, with a compound of formula V,
X²-Q^{a}-L² V
wherein Q^{a} represents a single bond or —C(O)—, L² represents a suitable leaving group and X² is as defined in claim 1;
(iii) for compounds of formula I in which X¹ represents -Q-X² and Q represents —C(O)—, reaction of a compound of formula I in which X¹ represents H with a compound of formula V in which Q^{a} represents —C(O)—;
(iv) for compounds of formula I in which X¹ represents —N(R⁹)-J-R¹⁰ or -Q-X in which Q represents —O— or —S—, reaction of a compound of formula IV as defined above with a compound of formula VI,
X^{1b}H VI
in which X^{1b} represents —N(R⁹)-J-R¹⁰ or -Q-X² in which Q represents —O— or —S— and R⁹, J, R¹⁰ and X² are as defined in claim 1;
(v) for compounds of formula I in which X¹ represents -Q-X² and Q represents —S—, reaction of a compound of formula I in which X represents H, with a compound of formula VI in which X^{1b} represents -Q-X², Q represents —S— and X² is as defined in claim 1;
(vi) for compounds of formula I in which X¹ represents -Q-X² and Q represents —S(O)— or —S(O)₂—, oxidation of a corresponding compound of formula I in which Q represents —S—;
(vii) for compounds of formula I in which X¹ represents -Q-X², X² represents C₁₋₈ alkyl substituted by G¹, G¹ represents -A¹-R^{11a}, A¹ represents —N(R^{2a})A⁴- and A⁴ is a single bond (provided that Q represents a single bond when X² represents substituted C₁ alkyl), reaction of a compound of formula VII, 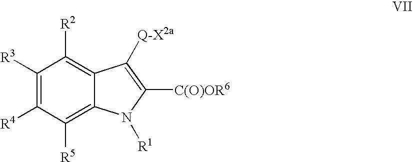
wherein X^{2a} represents a C₁₋₈ alkyl group substituted by a -Z¹, group in which Z¹ represents ═O, Q is as defined in claim 1, provided that it represents a single bond when X^{2a} represents C₁ alkyl substituted by ═O, and R¹, R², R³, R⁴, R⁵ and R⁶ are as defined in claim 1 under reductive amination conditions in the presence of a compound of formula VIII,
R^{11a}(R^{12a})NH VIII
wherein R^{11a} and R^{12a} are as defined in claim 1;
(viia) for compounds of formula I in which X¹ represents -Q-X², Q represents a single bond, X² represents methyl substituted by G¹, G¹ represents -A¹-R^{11a}, A¹ represents —N(R^{12a})A⁴- and A⁴ is a single bond, reaction of a corresponding compound of formula I in which X¹ represents H, with a mixture of formaldehyde and a compound of formula VIII as defined above;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula IV in which L¹ represents halo with a compound of formula IXA,
H₂C═C(H)X^{2b} IXA
or reaction of a compound of formula VII in which Q represents a single bond and X^{2a} represents —CHO with either a compound of formula IXB,
(EtO)₂P(O)CH₂X^{2b} IXB
or the like, or a compound of formula IXC,
(Ph)₃P═CHX^{2b} IXC
or the like, wherein, in each case, X^{2b} represents H, G¹ or C₁₋₆ alkyl optionally substituted with one of more substituents selected from G¹ and/or Z¹ and G¹ and Z¹ are as defined in claim 1;
(ix) for compounds of formula I in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction of a corresponding compound of formula I in which X² represents optionally substituted C₂₋₈ alkenyl, cycloalkenyl, heterocycloalkenyl, C₂₋₈ alkynyl, cycloalkynyl or heterocycloalkynyl (as appropriate);
(x) for compounds of formula I in which one or more of R², R³, R⁴ and/or R⁵ represents -D-E, in which D represents —C(O)—, —C(R⁷)(R⁸)—, C₂₋₄ alkylene or —S(O)₂—, or optionally substituted cycloalkyl or heterocycloalkyl, reaction of a compound of formula X, 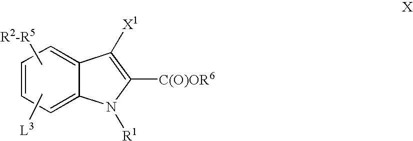
wherein L³ represents L¹ or L² as defined above, which group is attached to one or more of the carbon atoms of the benzenoid ring of the indole, R²-R⁵ represents whichever of the three other substituents on the benzenoid ring are already present in that ring, and X¹, R¹, R², R³, R⁴, R⁵ and R⁶ are as defined in claim 1, with, in the case where one or more of R² to R⁵ represents -D-E, in which D represents
—C(O)—, —C(R⁷)(R⁸)—, C₂₋₄ alkylene or —S(O)₂—, a compound of formula XI,
E-D^{a}-L⁴ XI
wherein D^{a} represents —C(O)—, —C(R⁷)(R⁸)—, C₂₋₄ alkylene or —S(O)₂—, L⁴ represents L¹ (when L³ is L²) or L² (when L³ is L¹), E, R⁷ and R⁸ are as defined in claim 1 and L¹ and L² are as defined above or, in the case where one of R² to R⁵ represents an optionally substituted cycloalkyl or heterocycloalkyl group, a compound of formula XIA,
(R²⁻⁵)-L⁴ XIA
wherein (R²⁻⁵) represents whichever one of the substituents R², R³, R⁴ or R⁵ is being introduced and R², R³, R⁴ and R⁵ are as defined in claim 1 and L⁴ is as defined above;
(xi) for compounds of formula I in which when one of R² to R⁵ represents -D-E- and D represents —S—, —O— or C₂₋₄ alkynylene in which the triple bond is adjacent to E, reaction of a compound of formula X as defined above in which L³ represents L² as defined above with a compound of formula XII,
E-D^{b}-H XII
wherein D^{b} represents —S—, —O— or C₂₋₄ alkynylene in which the triple bond is adjacent to E and E is as defined in claim 1;
(xii) for compounds of formula I in which when one of R² to R⁵ represents -D-E- and D represents —S(O)— or —S(O)₂—, oxidation of a corresponding compound of formula I in which D represents —S—;
(xiii) for compounds of formula I in which when one of R² to R⁵ represents -D-E- and D represents —O— or —S—, reaction of a compound of formula XIII, 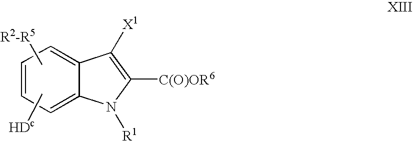
wherein the -D^{c}-H group is attached to one or more of the carbon atoms of the benzenoid ring of the indole, D^{c} represents —O— or —S— and X¹, R¹ and R⁶ are as defined in claim 1, and R²-R⁵ is as defined above, with a compound of formula XIV,
E-L² XIV
wherein L² is as defined above and E is as defined in claim 1;
(xiv) for compounds of formula I in which X¹ represents —N(R⁹)-J-R¹⁰, reaction of a compound of formula XV, 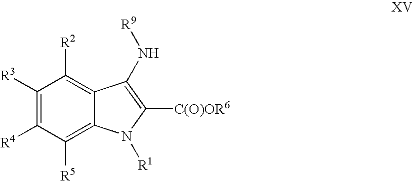
wherein R¹, R², R³, R⁴, R⁵, R⁶ and R⁹ are as defined in claim 1, with a compound of formula XVI,
R¹⁰-J-L¹ XVI
wherein J and R¹⁰ are as defined in claim 1 and L¹ is as defined above;
(xv) for compounds of formula I in which X¹ represents —N(R⁹)-J-R¹⁰, J represents a single bond and R¹⁰ represents a C₁₋₈ alkyl group, reduction of a corresponding compound of formula I, in which J represents —C(O)— and R¹⁰ represents H or a C₁₋₇ alkyl group, in the presence of a suitable reducing agent;
(xvi) for compounds of formula I in which X¹ represents halo, reaction of a compound of formula I wherein X¹ represents H, with a reagent or mixture of reagents known to be a source of halide atoms;
(xvii) for compounds of formula I in which R⁶ is other than H, reaction of a compound of formula XVII, 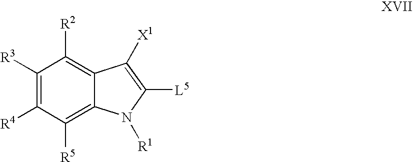
wherein L⁵ represents an appropriate alkali metal group, a —Mg-halide, a zinc-based group or a suitable leaving group and X¹, R¹, R², R³, R⁴ and R⁵ are as defined in claim 1, with a compound of formula XVIII,
L⁶C(O)OR^{6a} XVIII
wherein R^{6a} represents R⁶ provided that it does not represent H, and L⁶ represents a suitable leaving group;
(xviii) for compounds of formula I in which R⁶ is H, reaction of a compound of formula XVII in which L⁵ represents either:
(I) an alkali metal; or
(II)—Mg-halide,
with carbon dioxide, followed by acidification;
(xix) reaction of a compound of formula XVII in which L⁵ is a suitable leaving group with CO (or a reagent that is a suitable source of CO), in the presence of a compound of formula XIX,
R⁶OH XIX
wherein R⁶ is as defined in claim 1, and an appropriate catalyst system;
(xx) for compounds of formula I in which R⁶ represents H, hydrolysis of a corresponding compound of formula I in which R⁶ does not represent H;
(xxi) for compounds of formula I in which R⁶ does not represent H:
(A) esterification of a corresponding compound of formula I in which R⁶ represents H; or
(B) trans-esterification of a corresponding compound of formula I in which R⁶ does not represent H (and does not represent the same value of R⁶ as the compound of formula I to be prepared),
in the presence of the appropriate alcohol of formula XIX as defined above but in which R⁶ represents R^{6a};
(xxii) for compounds of formula I in which X¹ represents -Q-X² in which Q represents —O—, reaction of a compound of formula XX, 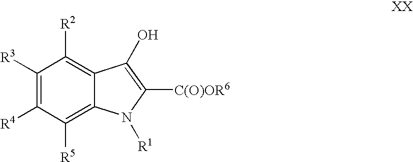
wherein R¹, R², R³, R⁴, R⁵ and R⁶ are as defined in claim 1, with a compound of formula XXI,
X²L⁷ XXI
wherein L⁷ represents a suitable leaving group and X² is as defined in claim 1;
(xxiii) for compounds of formula I in which X¹ represents —N(R⁹)-J-R¹⁰, reaction of a compound of formula XX as defined above, with a compound of formula VI in which X^{1b} represents —N(R⁹)-J-R¹⁰ and R⁹, R¹⁰ and J are as defined in claim 1;
(xxiv) for compounds of formula I in which X¹ represents -Q-X², Q represents a single bond and X² represents C₁₋₈ alkyl or heterocycloalkyl substituted a to the indole ring by a G¹ substituent in which G¹ represents -A¹-R^{11a}, A¹ represents —OA⁵-, A⁵ represents a single bond and R^{11a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O;
(xxv) for compounds of formula I in which X¹ represents -Q-X², Q represents a single bond and X² represents C₂₋₈ alkyl substituted by a G¹ substituent in which G¹ represents -A¹-R^{11a}, A¹ represents —OA⁵-, A⁵ represents a single bond and R^{11a} represents H, reaction of a corresponding compound of formula I in which X² represents C₁₋₇ alkyl substituted by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl;
(xxvi) for compounds of formula I in which X¹ represents -Q-X², Q represents a single bond, and X² represents C₁₋₈ alkyl or heterocycloalkyl, both of which are unsubstituted in the position α to the indole ring, reduction of a corresponding compound of formula I in which X² represents C₁₋₈ alkyl substituted α to the indole ring by a G¹ substituent in which G¹ represents -A¹-R^{11a}, A¹ represents
—OA⁵-, A⁵ represents a single bond and R^{11a} represents H;
(xxvii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O; or
(xxviii) for compounds of formula I in which one of the groups R², R³, R⁴ or R⁵ represents a heterocycloalkyl group linked to the benzenoid moiety of the indole ring by a nitrogen atom, reaction of a compound of formula X as defined above with a compound of formula XXIA,
(R^{2y-5y})H XXIA
wherein (R^{2y-5y}) represents R²⁻⁵ as defined above provided that the appropriate R², R³, R⁴ or R⁵ substituent represents a heterocycloalkyl group in which the hydrogen atom of the compound of formula XXIA is attached to a nitrogen atom of that group.
